(19) United States
(12) Patent Application Publication
(10) Pub. No.: US 2022/0274997 A1
(43) Pub. Date: Sep. 1, 2022

(54) PIM KINASE INHIBITOR COMPOSITIONS AND USES THEREOF

(71) Applicant: Snap Bio, Inc., San Diego, CA (US)

(72) Inventors: Mark J. Burk, San Diego, CA (US); Brandon Chen, San Diego, CA (US)

(21) Appl. No.: 17/607,325

(22) PCT Filed: Apr. 29, 2020
(86) PCT No.: PCT/US2020/030405

§ 371 Date: Oct. 28, 2021

Related U.S. Application Data
(60) Provisional application No. 62/840,095, filed on Apr. 29, 2019.

(51) Int. Cl. C07D 487/14; C07D 471/22; A61P 35/00

(52) U.S. Cl. CPC C07D 487/14; A61P 35/00; C07D 471/22

(57) ABSTRACT

This disclosure relates to compounds and compositions useful as inhibitors of PIM kinases. Also provided are methods of synthesis and methods of use of PIM inhibitors in treating individuals suffering from cancerous malignancies.

### BACKGROUND

Protein kinases mediate intracellular signaling by causing a phosphoryl transfer from a nucleoside triphosphate to a protein acceptor that is involved in a signaling pathway. These phosphorylation events act as molecular on/off switches that can modulate or regulate the target protein biological function.

Many diseases are associated with abnormal cellular responses triggered by protein kinase-mediated events as described above (Roskoski, R. Jr., Pharmacol. Res., 2015, 100, 1-23; Fleuren, E. D. G., et al., Nat. Rev. Cancer, 2016, 16, 83-98). These diseases include, but are not limited to, cancer, autoimmune diseases, inflammatory diseases, bone diseases, metabolic diseases, neurological and neurodegenerative diseases, cardiovascular diseases, allergies and asthma, Alzheimer's disease, and hormone-related diseases. Accordingly, there remains a need to find new safe and efficacious protein kinase inhibitors that are useful as therapeutic agents.

Proviral integration site for Moloney murine leukemia virus (PIM) kinases area family of three constitutively active proto-oncogenic serine/threonine kinases, PIM1, PIM2, and PIM3, which have been shown to regulate signaling associated with several important normal biological processes, including cell survival, proliferation, differentiation, and apoptosis. However, when these processes become disrupted or hyperactivated, they manifest the hallmarks of cancer. The PIM kinases promote cell survival and downregulate cell apoptosis and, accordingly, have been shown to be involved directly in signaling mechanisms associated with tumorigenesis.

Due to the poor prognosis and limited therapeutic options associated with endodermal and other cancers that overexpress PIM1 and/or PIM3, development of selective inhibitors of PIM1 and/or PIM3 kinase represents a novel strategy for specifically treating cancers, especially sarcomas such as Ewing's sarcoma, or cancers of the stomach, liver, colon, prostate, esophagus, pancreas, and other endodermal organs, with drugs, either alone or in combination with other therapeutic agents, that exhibit higher efficacy, lower toxicity, and lower susceptibility to resistance relative to existing treatment modalities.

### SUMMARY

This disclosure provides potent and selective inhibitors of the family of three PIM inhibitor compositions of general Formulas (I) set out below, pharmaceutical formulations, methods for their preparation, and uses thereof, including uses aimed at specifically targeting endodermal cancers through selective inhibition of PIM3.

A disclosed embodiment is a compound having the structure of Formula (I) or a pharmaceutically acceptable salt, solvate, hydrate, N-oxide, prodrug, stereoisomer, enantiomer, mixture of enantiomers, mixture of diasteromers, isotopic variants, and metabolites thereof; wherein Formula (I) is as defined.

Other embodiments are compounds having the structure of Formulas (IIA) or (IB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (IIA) or (IIB) are as defined below.

Other embodiments are compounds having the structure of Formulas (IIIA) or (IIIB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (IRA) or (IB) are as defined i below.

Other embodiments are compounds having the structure of Formulas (VA) or (IVB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (IVA) or (IVB) are as defined below.

Other embodiments are compounds having the structure of Formulas (VA) or (VB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (VA) or (VB) am as defined in below.

Other embodiments are compounds having the structure of Formulas (VIA) or (VIB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (VIA) or (VIB) are as defined below.

Other embodiments are compounds having the structure of Formulas (VIIA) or (VIIB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (VIIIA) or (VIIIB) are as defined below.

Other embodiments are compounds having the structure of Formulas (IXA) or (IXB) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein Formulas (IXA) or (IXB) are as defined in below.

Other embodiment of this disclosure are compounds having the structure of Formulas (IXA) or (IXB) or a pharmaceutically acceptable salt, solvate, hydrate, N-oxide, prodrug, stereoisomer, enantiomer, mixture of enantiomers, mixture of diastereomers, isotopic variants, and metabolites thereof, which we examples representing kinase inhibitors, wherein Formulas (IXA) or (IXB) are as defined Description below.

Also disclosed we pharmaceutical compositions comprising a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof; and one or more pharmaceutically acceptable excipients.

Further disclosed is a method of treating, preventing, or ameliorating one or more symptoms of a disorder, disease, or condition involving PIM3 expression in a subject, comprising administering to the subject a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof.

Also disclosed is a method of treating, preventing, or ameliorating one or more symptoms of a disorder, disease, or condition in a subject, including cancers of endodermal organs such as the stomach, liver, colon, pancreas, prostate, and gallbladder, as well as other cancers involving PIM3 expression, comprising administering to the subject a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof.

Additionally, disclosed is a method of modulating PIM3 kinase activity, comprising contacting a PIM3 kinase in vitro or in vivo with a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof.

Also disclosed is a method of treating, preventing, or ameliorating one or more symptoms of a PIM3 kinase-mediated disorder, disease, or condition in a subject, comprising administering to the subject a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof, wherein the compound partially or completely inhibits PIM3 activity and displays increased potency against PIM3 kinase and/or increased selectivity for inhibiting PIM3 kinase relative to PIM1 kinase, PIM2 kinases and other kinases known to be present in the human body.

Also disclosed are methods for treating cancerous conditions characterized by, for example, abnormal fatigue, pain, persistent lumps, bleeding, stiffness, dizziness, anemia, susceptibility to infection, persistent cough or itching, headaches, sudden weight loss, nonhealing sores, and fever. Cancerous malignancies can affect almost any part of the body, including the heart, brain, nerves, muscles, skin, eyes, joints, lungs, pancreas, prostate, reproductive organs, kidneys, glands, lymphatic system, immune system, gastrointestinal system, circulatory system and blood vessels.

### BRIEF DESCRIPTION OF THE DRAWINGS

FIGS. 1A, 1B and 1C are graphical representations of IC₅₀ plots for Compound 18 targeting PIM1-3 using the biochemical assay and showing PIM3 selectivity.

FIGS. 2A and 2B are graphical representations of EC₅₀ plots for Compounds 32 and 53, respectively, targeting PIM1-3 kinases using the LANCE Ultra assay, showing PIM1 and PIM3 selectivity.

FIGS. 3A, 3B and 3C are graphical representations of EC₅₀ cytotoxicity plots for Compounds 19, 24, and 37 exhibiting growth inhibition when added to cancer cell lines HepG2, A673, and Huh7, respectively.

FIG. 4 is a graphical representation of relative Hepa1-6 tumor size in CD57 mice upon dosing with Compound 49.

### DETAILED DESCRIPTION

To facilitate an understanding of the disclosure set forth herein, a number of terms are defined below.

Generally, the nomenclature used herein and the laboratory procedures in organic chemistry, medicinal chemistry, molecular biology, microbiology, biochemistry, enzymology, computational biology, computational chemistry, and pharmacology described herein are those well-known and commonly employed in the art. Unless defined otherwise, all technical and scientific terms used herein generally have the same meaning as commonly understood by one of ordinary skill in the art to which this disclosure belongs.

The following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 6^{th} Ed., Ed.: Smith, M. B. and March, J., John Wiley & Sons, New York: 2007, the entire contents of which are hereby incorporated by reference.

Provided herein are compounds and methods for the treatment of certain forms of cancer by administration of selective inhibitors of PIM kinases to subjects in need thereof. In certain embodiments, the subject has been diagnosed with or is suspected of suffering from a cancer that is found to be associated with the overexpression or hyperactivity PIM kinases. Cancers of the endodermal organs, such as the stomach, liver, colon, pancreas and gallbladder, represent examples of cancers where PIM1 and/or PIM3 involvement has been demonstrated, and thus are candidates for treatment with an efficacious PIM1 and/or PIM3 inhibitor.

### PIM Inhibitors

Disclosed are inhibitors of the PIM kinases, with certain compounds exhibiting selective inhibition of PIM1 and/or PIM3. Highly selective PIM1 and/or PIM3 inhibitors are useful for specifically treating cancers that express and/or depend on the activity of this kinase for its pathological growth and proliferation. Endodermal cancers, including malignancies of the stomach, colon, liver, pancreas, prostate, and gallbladder, have been shown to overexpress PIM1 and/or PIM3 and inhibition of PIM1 and/or PIM3 has been demonstrated to inhibit growth of these cancers. Also described herein are pharmaceutical compositions comprising PIM inhibitors (e.g., PIM inhibitor compounds described herein) for reversing or reducing one or more of the negative symptoms associated with cancerous malignancies, including endodermal cancers. Also described herein are pharmaceutical compositions comprising PR inhibitors for halting or delaying the progression of negative symptoms associated with cancerous malignancies, including endodermal cancers. Described herein is the use of a PIM inhibitor for manufacture of a medicament for treatment of one or more symptoms of cancer.

In some embodiments, the PIM inhibitors described herein inhibit all PIM kinases with approximately equal potency. In certain embodiments, a PIM inhibitor described herein reduces or inhibits the activity of one or more of PIM kinases while largely not affecting the activity of the others. In some embodiments, a PIM inhibitor described herein substantially reduces or inhibits the kinase activity of PIM3. In some embodiments, a PIM inhibitor described herein is a substantially complete inhibitor of the PIM3 kinase. As used herein, “substantially complete inhibition” means, for example, >95% inhibition of PIM3. In other embodiments, “substantially complete inhibition” means, for example, >90% inhibition of PIM3. In some other embodiments, “substantially complete inhibition” means, for example, >80% inhibition of PIM3. In some embodiments, a PIM3 inhibitor described herein is a partial inhibitor of PIM3. As used herein, “partial inhibition” means, for example, between about 40% to about 60% inhibition of PIM3. In other embodiments, “partial inhibition” means, for example, between about 50% to about 70% inhibition of PIM3. As used herein, where a PIM3 inhibitor substantially inhibits or partially inhibits the activity of PIM3 while largely not affecting the activity of PIM1 and/or PIM2, it means, for example, less than about 10% inhibition of PIM1 and/or PIM2 when PIM1 and/or PIM2 are contacted with the same concentration of the PIM3 inhibitor. In other instances, where a PIM3 inhibitor substantially inhibits or partially inhibits the activity of PIM3 while not affecting the activity of PIM1 and/or PIM2, it means, for example, less than about 5% inhibition of PIM and/or PIM2 when PIM1 and/or PIM2 are contacted with the same concentration as used for PIM3. In yet other instances, where a PIM3 inhibitor substantially inhibits or partially inhibits the activity of PIM3 while largely not affecting the activity of PIM and/or PIM2, it means, for example, less than about 1% inhibition of PIM1 and/or PIM2 when PIM and/or PIM2 is contacted with the same concentration of the PIM3 inhibitor as used for PIM3.

One embodiment is compounds having the structure of Formula (I) or a pharmaceutically acceptable salt, solvate, hydrate, N-oxide, prodrug, stereoisomer, enantiomer, mixture of enantiomers, mixture of diastereomers, isotopic variants, and metabolites thereof wherein:

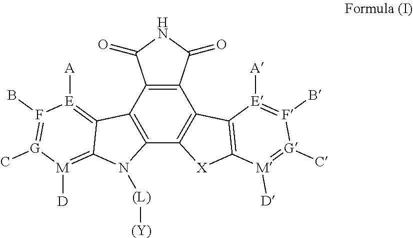

Each A, B, C And D is the same or different and independently selected from H, halogen, —N₃, —CN, —NO₂, —OH, —OCF₃, —OCH₂F, —OCF₂H, —CF₃, —CF₂H, —SR¹, —S(═O)R², —S(═O)₂R₂, —OS(═O)₂F, —OS(═O)₂(OR²), —S(═O)₂(OR²), —NR³S(═O)₂R₂, —S(═O)₂N(R³)₂, —OC(═O)R², —CO₂R³, —OR³, —N(H)R³, —N(R³)₂, —NR³C(═)R₂, —NR³C(═O)OR³, —NR³C(═O)N(R³)₂, CH₂NH₂, —CH₂N(R³)₂, —CH₂SR¹, —C(═O)NH₂, —C(═O)N(R³)₂, —C(═O)R³, substituted or unsubstituted alkyl, substituted or unsubstituted alkenyl, substituted or unsubstituted alkynyl, substituted or unsubstituted alkoxy, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an optional substituent selected, for example, haloalkyl, alkenyl, arylalkyl, alkoxyalkyl, hydroxyalkyl, monoalkylaminoalkyl, dialkylaminoalkyl, acylaminoalkyl, acyloxyalkyl, cyanoalkyl, amidinoalkyl, carboxyalkyl, alkdxycarbonylalkyl, aminocarbonylalkyl, aryl, alkylaryl, aminoalkyl, heteroaryl, carbonylalkyl, amidinothioakyl, nitroguanidinoalkyl, a protecting group, a glycose, aminoglycose or alkylgyoose residue;

Each A′, B′, C′, and D′ is the same or different and independently selected from H, halogen, —N₃, —CN, —NO₂, —OH, —OCF₃. —OCH₂F, —OCF₂H, —CF₃, —CF₂H, —SR¹, —S(═O)R², —S(═O)₂R², —OS(═O)₂F, —OS(═O)₂(OR²), —S(═O)₂(OR²), —NR³S(═O)₂R², —S(═O)₂N(R³)₂, —OC(═O)R², —CO₂R³, —N(H)R³, —N(R)₂, —OR³, —NR³C(═O)R², —NR³C(═O)OR³, —NR³C(═O)N(R³)₂, CH₂NH₂, —CH₂N(R³), —CH₂SR¹, —C(═O)NH₂, —C(O)N(R³)₂, —C(═O)R³, substituted or unsubstituted alkyl, substituted or unsubstituted alkenyl, substituted or unsubstituted alkynyl, substituted or unsubstituted alkoxy, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an optional substituent selected, for example, haloalkyl, alkenyl, arylalkyl, alkoxyalkyl, hydroxyalkyl, monoalkylaminoalkyl, dialkylaminoalkyl, acylaminoalkyl, acyloxyalkyl, cyanoalkyl, amidinoalkyl, carboxyalkyl, alkoxycarbonylalkyl, aminocarbonylalkyl, aryl, alkylaryl, aminoalkyl, heteroaryl, carbonylalkyl, amidinothioalkyl, nitroguanidinoalkyl, a protecting group, a glycose, aminoglycose or alkylglycose residue;

Each E, F, G, and M is independently C or N;

Each E′, F′, G′, and M′ is independently C or N;

Each Y is a monosubstituted, disubstituted, or cyclic amine group;

Each L is a linear alkyl chain of 1-6 carbons directly linked to an amine N atom of Y;

Each X is NH, O, S, or CH₂;

R¹ is H or linear or branched substituted or unsubstituted alkyl, substituted or unsubstituted alkenyl, substituted or unsubstituted cycloalkyl, substituted or unsubstituted cycloalkenyl, substituted or unsubstituted heterocycloalkyl, substituted or unsubstituted aryl or substituted or unsubstituted heteroaryl;

R² is linear or branched substituted or unsubstituted alkyl, substituted or unsubstituted alkenyl, substituted or unsubstituted cycloalkyl, substituted or unsubstituted cycloalkenyl, substituted or unsubstituted heterocycloalkyl, substituted or unsubstituted aryl or substituted or unsubstituted heteroaryl;

Each R³ is independently H, linear or branched substituted or unsubstituted alkyl. substituted or unsubstituted alkenyl, substituted or unsubstituted cycloalkyl, substituted or unsubstituted cycloalkenyl, substituted or unsubstituted heterocycloalkyl substituted or unsubstituted aryl, substituted or unsubstituted heteroaryl, or substituted or unsubstituted acyl (—C(═O)R¹), or two R³ together with the atoms to which they are attached form a substituted or unsubstituted heterocycle;

Compounds of Formula (I) are themselves useful as protein kinase inhibitors or represent intermediates useful for the preparation of compounds exhibiting kinase Inhibitory activity. As noted above, kinase Inhibitors are useful for treating a variety of conditions including cancer, central nervous system disorders, Alzheimer's, cardiovascular disease, dermatological diseases, inflammation, autoimmune diseases such as rheumatoid arthritis, and diabetic complications.

Another embodiment is compounds having the structure of Formula (IIA), (IIB), or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein:

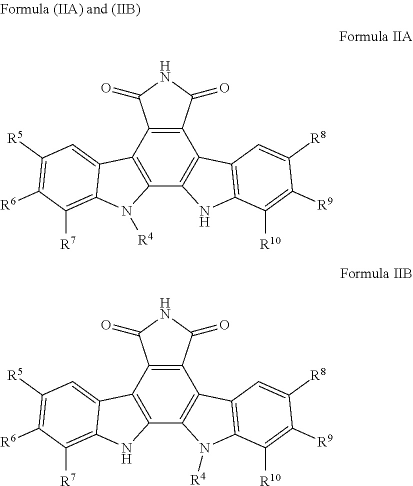

Each R⁴ is one of the following amine groups where n=0-5.

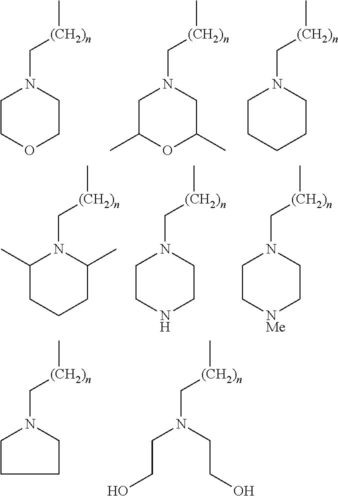

Each R⁵, R⁶, R⁷, R⁸, R⁹, and R¹⁰ is the same or different and independently selected from H, halogen, —N₃, —CN, —NO₂, —OH, —OCF₃, —OCH₂F, —OCF₂H, —CF₃, —CF₂H, —SR¹, —S(═O)R², —S(═O)R², —OS(═O)₂F, —OS(═O)₂(OR²), —S(═O)₂(OR²), —NR³S(═O)₂R, —S(═O)₂N(R³)₂, —OC(═O)R², —CO₂R³, —N(H)R³, —N(R³)₂, —OR³, —NR³C(═O)R², —NR³C(═O)OR³, —NRC(═O)N(R³)₂, CH₂NH₂, —CH₂N(R′)₂, —CH₂SR¹, —C(═O)NH₂, —C(═O)N(R³)₂, —C(═O)R³, substituted or unsubstituted alkyl, substituted or unsubstituted alkenyl, substituted or unsubstituted alkynyl, substituted or unsubstituted alkoxy, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an optional substituent selected, for example, haloalkyl, alkenyl, arylalkyl, alkoxyalkyl, hydroxyalkyl, monoalkylaminoalkyl, dialkylaminoalkyl, acylaminoalkyl, acyloxyalkyl, cyanoalkyl, amidinoalkyl, carboxyalkyl, alkoxycarbonylalkyl, aminocarbonylalkyl, aryl, alkylaryl, aminoalkyl, heteroaryl, carbonylalkyl, amidinothioalkyl, nitroguanidinoalkyl, a protecting group, a glycose, aminoglycose or alkylglycose residue.

Selected compounds of Formulas IIA and IIB comprise compounds of Formulas IIA and IIB wherein each R⁴ is selected from the group consisting of

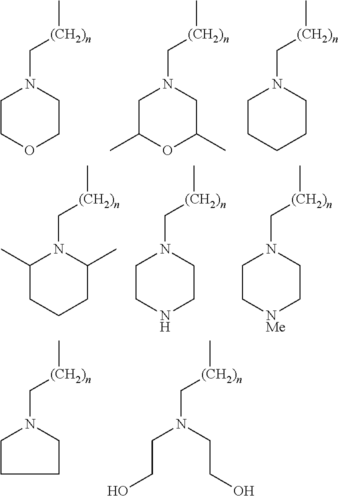

where n=1,

each R⁵, R⁶, R⁸, and R⁹ is the same or different and independently selected from H, halogen, or —OH, and each R⁷ and R¹⁰ is H.

Another embodiment is compounds having the structure of Formula (IIIA), (IIIB), or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein:

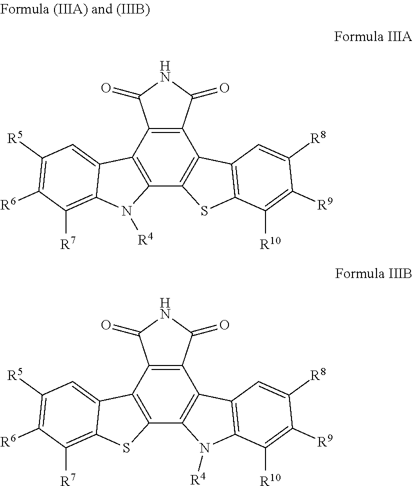

Each R⁴, R⁵, R⁶, R⁷, R⁸, R⁹ and R¹⁰ is as defined above for Formula (IIA).

Another embodiment is compounds having the structure of Formula (IVA), (IVB), or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein:

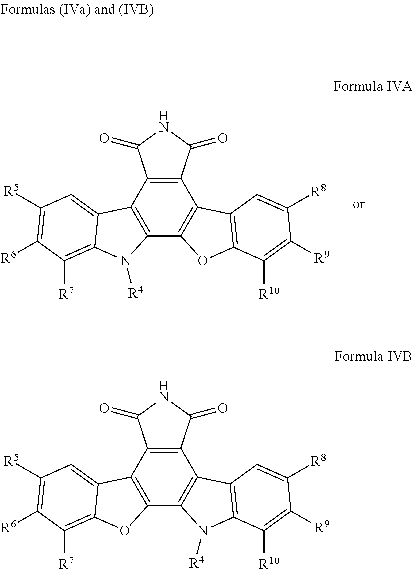

Each R⁴, R⁵, R⁶, R⁷, R⁸, R⁹ and R¹⁰ is as defined above for Formula (IIA).

Another embodiment is compounds having the structure of Formula (VA), (VB), or a pharmaceutically acceptable salt, solvate, hydrate, N-oxide, prodrug, stereoisomer, enantiomer, mixture of enantiomers, mixture of diasteromers, isotopic variants, and metabolites thereof, which are examples representing kinase inhibitors, wherein:

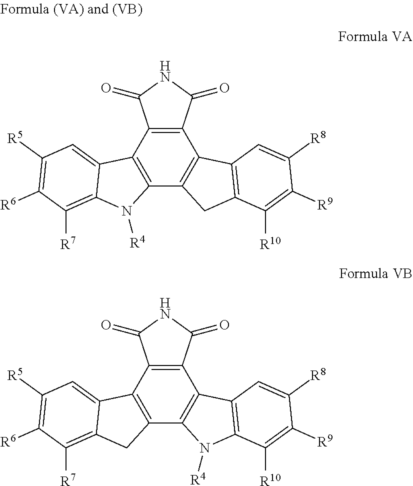

Each R⁴, R⁵, R⁶, R⁷, R⁸, R⁹ and R¹⁰ is as defined above for Formula (HA).

Another embodiment is compounds having the structure of Formula (VI) or a pharmaceutically acceptable salt, solvate, hydrate, N-oxide, prodrug, stereoisomer, enantiomer, mixture of enantiomers, mixture of diastereomers, isotopic variants, and metabolites thereof, which site examples representing kinase inhibitors, wherein:

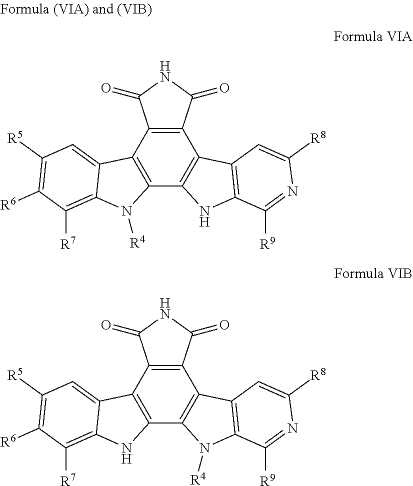

Each R⁴, R⁵, R⁶, R⁷, R⁸, and R⁹ as defined above for Formula (II).

Selected compounds of Formulas VIA and VIB comprise compounds of Formulas VIA and VIB wherein each R⁴ is selected from the group consisting of

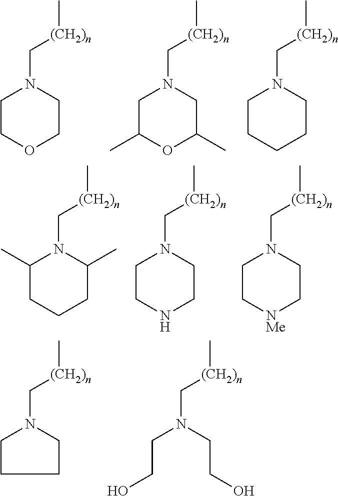

where n=1,

each R⁵, R⁶, and R⁸ is the same or different and independently selected from H, halogen, or —OH, and each, R⁷ and R⁹ is H,

Another embodiment is compounds having the structure of Formula (VII) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein:

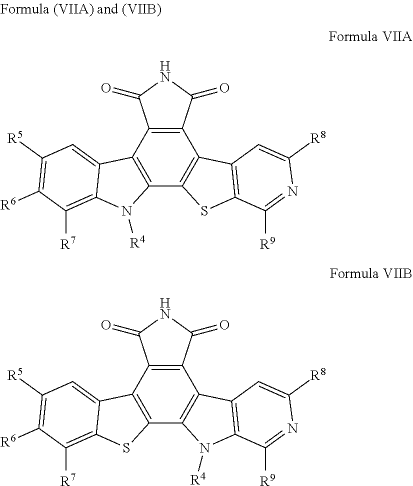

Each R⁴, R⁵, R⁶, R⁷, R⁸, and R⁹ is as defined above for Formula (II).

Another embodiment is compounds having the structure of Formula (VIII) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein:

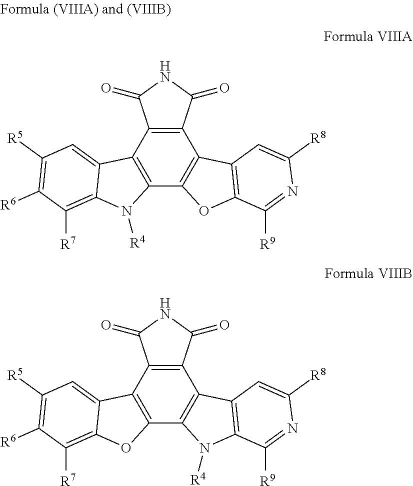

Each R⁴, R⁵, R⁶, R⁷, R⁸, and R⁹ is as defined above for Formula (II)

In another embodiment are compounds having the structure of Formula (IX) or a pharmaceutically acceptable salt, solvate, hydrate, N-oxide, prodrug, stereoisomer, enantiomer, mixture of enantiomers, mixture of diastereomers, isotopic variants, and metabolites thereof, which are examples representing kinase inhibitors, wherein:

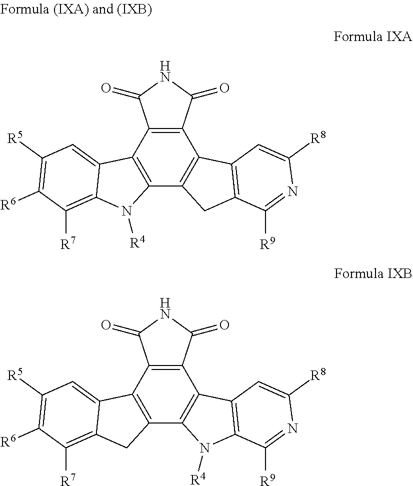

Each R⁴, R⁵, R⁶, R⁷, R⁸, and R⁹ is as defined above for Formula (II).

Another embodiment is compounds of Formulas (I)-(IX) wherein unsubstituted alkyl is selected from methyl, ethyl, n-propyl, iso-propyl, n-butyl, iso-butyl, tert-butyl, n-pentyl, n-hexyl, etc. In another embodiment, A, B, C, and D each are independently H, F, Cl, Br, I, —OH, —CN, —N₃, —OR³, —NO₂, —NH₂, —CH₂NH₂, —CH₂N(R³)₂, —CH₂SR¹, —C(═O)NH₂, —C(═O)N(R³)₂, —C(═O)R³, substituted 1,2,3-triazole, substituted or unsubstituted aryl, substituted or unsubstituted heteroaryl, substituted or unsubstituted alkenyl, substituted or unsubstituted alkynyl, or substituted or unsubstituted acyl; In another embodiment, A′, B′, C′, and D′ are independently H, F, Cl, Br, I, —OH, —CN, —N₃, —OR³, —NO₂, —NH₂, —CH₂NH, —CH₂N(R³)₂, —CH₂SR¹, —C(═O)NH₂, —C(═O)N(R³)₂, —C(═O)R³, substituted 1,2,3-triazole, substituted or unsubstituted aryl, substituted or unsubstituted heteroaryl, substituted or unsubstituted alkenyl, substituted or unsubstituted alkynyl, or substituted or unsubstituted acyl; In another embodiment, E, F, G, or M are independently nitrogen. In another embodiment, E′, F′, G′, or M′ are independently nitrogen. In another embodiment, E and F are nitrogen. In another embodiment, E′ and F′ are nitrogen. In another embodiment, E and F are nitrogen. In another embodiment, E and G are nitrogen. In another embodiment, E′ and G′ are nitrogen.

In another embodiment, E and M are nitrogen. In another embodiment, E′ and M′ are nitrogen. In another embodiment, F and M are nitrogen. In another embodiment, F′ and M′ are nitrogen.

In another embodiment of this disclosure are compounds of Formula (U) wherein Y, Z, Y′, and Z′ are hydrogen, and X is —CH₂N(Me)₂.

In alternative embodiments, provided herein are methods to produce bisindole alkaloids and analogs of Formulas (I)-(IX) through a coupled transcription/translation (TX-TL) cell-free biosynthesis (CFB) system, wherein reactions are conducted by adding bisindole alkaloid pathway genes to cell-free extracts containing metabolic enzymes, salts, co-factors, amino acids, sugars, nucleotides, and precursor molecules such as tryptophan and/or tryptophan derivatives, and wherein optionally the mixture is capable of in vitro transcription, translation and/or coupled transcription/translation to produce molecules of Formula (I) where Q and R independently are either hydrogen, a glycose group attached to one indole nitrogen atom, or together form a glycose that bridges both indole nitrogen atoms. Compounds of Formula (I) subsequently may be produced through chemical transformations that introduce non-hydrogen Q and R groups.

In alternative embodiments, cell-free extracts are created by growing and breaking open cells, removing cell membrane and cell wall materials, and digesting native DNA and/or RNA, wherein the cells derive from different kingdoms, phyla, classes, orders, families, genera or species and the cells are a prokaryotic or a eukaryotic cell; or, a bacterial cell, a fungal cell, an algae cell, an Archaeal cell, a yeast cell, an insect cell, a plant cell, a mammalian cell or a human cell.

In alternative embodiments, provided herein are methods to produce bisindole alkaloids and analogs of Formulas (I)-(IX) through cell-free reactions involving the use of isolated enzymes corresponding to the natural or unnatural pathway enzymes for bisindole alkaloid synthesis, wherein tryptophan and/or tryptophan derivatives are combined with such enzymes to afford molecules of Formulas (I)-(IX).

Also provided herein are pharmaceutical compositions comprising a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof; and one or more pharmaceutically acceptable excipients.

Further provided herein is a method of treating, preventing, or ameliorating one or more symptoms of a disorder, disease, or condition involving PIM3 expression in a subject, comprising administering to the subject a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof.

Also provided herein is a method of treating, preventing, or ameliorating one or more symptoms of a disorder, disease, or condition in a subject, including cancers of endodermal organs such as the stomach, liver, colon, pancreas, prostate, and gallbladder, as well as other cancers involving PIM3 expression, comprising administering to the subject a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof.

Additionally provided herein is a method of modulating PIM kinase activity, comprising contacting a PIM kinase in vitro or in vivo with a therapeutically effective amount of a compound disclosed herein, e.g., a compound of Formulas (I)-(IX) including a stereoisomer, enantiomer, mixture of enantiomers, mixture of diastereomers, or isotopic variant thereof; or a pharmaceutically acceptable salt, solvate, hydrate, or prodrug thereof.

Also provided herein is a method of treating, preventing, or ameliorating one or more symptoms of a PIM kinase-mediated disorder, disease, or condition in a subject, comprising administering to the subject a therapeutically effective amount of a compound disclosed herein, e.g., a compound of Formulas (I)-(IX), including a stereoisomer, enantiomer, mixture of enantiomers, mixture of diastereomers, or isotopic variant thereof; or a pharmaceutically acceptable salt, solvate, hydrate, or prodrug thereof, wherein the compound partially or completely inhibits PIM activity and displays increased potency against one PIM kinase and/or increased selectivity for inhibiting one PIM kinase relative to the other two PIM kinases and relative to other kinases known to be present in the human body.

In some embodiments, a PIM inhibitor is a small molecule. As referred to herein, a “small molecule” is an organic molecule that is less than about 5 kilodaltons (kDa) in size. In some embodiments, the small molecule is less than about 4 kDa, 3 kDa, about 2 kDa, or about 1 kDa. In some embodiments, the small molecule is less than about 800 daltons (Da), about 600 Da, about 500 Da, about 400 Da, about 300 Da, about 200 Da, or about 100 Da. In some embodiments, a small molecule is less than about 4000 g/mol, less than about 3000 g/mol, 2000 g/mol, less than about 1500 g/mol, less than about 1000 g/mol, less than about 800 g/mol, or less than about 500 g/mol. In some embodiments, small molecules are non-polymeric. Typically, small molecules are not proteins, polypeptides, polynucleotides, oligonucleotides, polysaccharides, glycoproteins, or proteoglycans, but include peptides of up to about 40 amino acids. A derivative of a small molecule refers to a molecule that shares the same structural core as the original small molecule, but which is prepared by a series of chemical reactions that vary and form a derivative of the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule. In certain embodiments, compounds described herein have one or more chiral centers. As such, all stereoisomers are envisioned herein. In various embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieve in any suitable manner, including by way of non-limiting example, by resolution of the racemic form by recrystallization techniques, by synthesis tom optically-active starting materials, by chiral synthesis, or by chromatographic separation using a chiral stationary phase. In some embodiments, mixtures of one or more isomer are utilized as the therapeutic compound described herein. In certain embodiments, compounds described herein contain one or more chiral centers. These compounds are prepared by any means, including enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, chromatography, and the like.

In various embodiments, pharmaceutically acceptable salts described herein include, by way of non-limiting example, a nitrate, chloride, bromide, phosphate, sulfate, acetate, hexafluorophosphate, citrate, gluconate, benzoate, propionate, butyrate, sulfosalicylate, maleate, laurate, malate, fumarate, succinate, tartrate, amsonate, pamoate, p toluenesulfonate, mesylate and the like. Furthermore, pharmaceutically acceptable salts include, by way of non-limiting example, alkaline earth metal salts (e.g., calcium or magnesium), alkali metal salts (e.g., sodium-dependent or potassium), ammonium salts and the like.

Compounds described herein also include isotopically-labeled compounds wherein one or more atoms is replaced by an atom having the se atomic number, but an atomic mass or mass number different from the atomic mass or mass number usually found in nature.

The compounds described herein, and other related compounds having different substituents are synthesized using techniques and materials described herein and/or as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989). March, ADVANCED ORGANIC CHEMISTRY 6th Ed., (Wiley 2007); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3′ Ed., (Wiley 1999), all of which are incorporated herein by reference for such disclosure. General methods for the preparation of compound as described herein are modified by the use of appropriate reagents and conditions, for the introduction of the various moieties found in the formula as provided herein.

### Definitions

The terms “halo” and “halogen” as used herein to identify substituent moieties, represent fluorine, chlorine, bromine or iodine, preferably fluorine, chlorine or bromine.

The term “alkyl” alone or in combination, represents a cyclic, linear or branched chain saturated hydrocarbon group, which in the case of straight and branched chains, preferably has from one to four carbon atoms (C₁-C₄ alkyl) and in the case of a cyclic hydrocarbon preferably has from three to seven carbon atoms. The term “substituted alkyl” is intended to include an alkyl group substituted with a substituent group that is not H. Reference to an alkyl group includes “saturated alkyl” and/or “unsaturated alkyl”. The alkyl group, whether saturated or unsaturated, includes branched, straight chain, or cyclic groups. A “lower alkyl” is a C₁-C₆ alkyl.

The term “cycloalkyl” refers to a cyclic hydrocarbon chain, wherein the cycloalkyl is optionally substituted with one or more substituents as described herein. In one embodiment, monocyclic or polycyclic cycloalkyl groups may be saturated or unsaturated, but non-aromatic, and/or spiro and/or non-spiro, and/or bridged, and/or non-bridged, and/or fused bicyclic groups, wherein each of the atoms forming the ring (i.e. skeletal atoms) is a carbon atom. In various embodiments, cycloalkyls are saturated. or partially unsaturated. In some embodiments, cycloalkyls are fused with an aromatic ring. The term cycloalkyl includes “unsaturated nonaromatic carbocyclyl” or “nonaromatic unsaturated carbocyclyl” groups both of which refer to a nonaromatic carbocycle, as defined herein that contains at least one carbon-carbon double bond or one carbon-carbon triple bond.

The term “haloalkyl” is one such substituted alkyl, substituted with one or more halo atoms, and preferably is a C₁ to C₁₀ alkyl substituted with one to five halo atoms. Examples of haloalkyl groups include, but are not limited to: difluoromethyl, dichloromethyl, trifluoromethyl, 2,2,2-trifluoroethyl, and pentafluoroethyl.

The term “alkoxy”, used alone or in combination, is an alkyl, preferably a C₁ to C₄ alkyl, covalently bonded to the parent molecule through an —O— linkage alone or in combination. Examples of alkoxy groups are methoxy, ethoxy, propoxy, isopropoxy, butoxy and t-butoxy. The team alkoxycarbonyl is, for example, t-butoxycarbonyl or BOC. An “alkoxy” group refers to a (alkyl)O— group, where alkyl is as defined herein.

The term “alkylamine” refers to the —N(alkyl)ₓH_{y} group, wherein alkyl is as defined herein and x and y are selected from the group x=1, y=1 and x=2, y=0. When x=2, the alkyl groups, taken together with the nitrogen to which they are attached, optionally form a cyclic ring system.

As used herein, the tam “aryl” refers to an aromatic ring wherein each of the atoms forming the ring is a carbon atom. Aryl rings described herein include rings having five, six, seven, eight, nine, or more than nine carbon atoms. Aryl groups are optionally substituted. Examples of aryl groups include, but are not limited to phenyl, and naphthalenyl. The term “aryl” when used alone or in combination represents a substituted or unsubstituted phenyl, biphenyl, or naphthyl. Aryl may optionally be substituted with one or more substituents that are independently selected from hydroxy, carboxy, alkoxy, preferably a C₁ to C₁₀ alkoxy, an alkyl, preferably a C₁-C₁₀ alkyl, a haloalkyl, nitro, —NR²R³, —NHCO(C₁-C₁₀ alkyl), —NHCO(benzyl), —NHCO(Phenyl), —SH, —S(C₁-C₄ alkyl), —(C₁-C₄ alkyl), —SO₂(NR²R³), —SO₂(C₁-C₁₀ alkyl), —SO₂ (phenyl), or halo wherein R² and are as defined above.

The term “aryloxy” is one such aryl covalently bonded through an —O— linkage. The term “arylalkyl” can be considered a substituted alkyl and represents —(CH₂)ₘaryl with m being an integer of generally 1 to 3, and preferably is benzyl. In contrast, the term alkylaryl can be considered a substituted aryl and may, for example, represent a moiety such as aryl(CH₂)ₙ—CH₃ where n is an integer of generally 0 to 6.

The term “alkenyl” refers to a two to term carbon, linear or branched hydrocarbon containing one or more carbon-carbon double bonds, preferably one or two double bonds, wherein the alkenyl group is optionally substituted with one or more substituents as described herein. Examples of alkenyl include ethylenyl, propylenyl, 1,3-butadienyl, and 1,3,5-hexatrienyl.

The term “alkynyl” refers to a linear or branched hydrocarbon, which contains one or more carbon-carbon triple bond(s), wherein the alkynyl is optionally substituted with one or more substituents as described herein.

The acyl moiety of an acylamino or arylaminoalkyl group is derived from an alkanoic acid containing a maximum of 10, preferably a maximum of 6, carbon atoms (e.g., acetyl, propionyl or butyryl) or from an aromatic carboxylic acid (e.g; benzoyl). An acyloxy is one such acyl bonded by an —O— linkage, for example, acetyloxy, CH₃C(═O)O—. An acylamino is, for example, CH₃(C═O)NH— (acetylamino). Likewise, an acylaminoalkyl is CH₃(C═O)NH(CH₂)ₘ—.

The term “heteroatom” refers to any atom that is not carbon or hydrogen, such as the halogens, phosphorus, nitrogen, oxygen, or sulfur, and includes any oxidized form of nitrogen or sulfur, and any quaternized form of a basic nitrogen. A “heteroalkyl” group substitutes any one of the carbons of the alkyl group with a heteroatom having the appropriate number of hydrogen atoms attached (e.g., a CH, group to an NH group or an O group).

An “amide” is a chemical moiety with formula —C(O)NHR or —NHC(O)R, where R is selected from alkyl, cycloalkyl, aryl, heteroaryl (bonded through a ring carbon) and heteroalicyclic (bonded through a ring carbon).

The term “ester” refers to a chemical moiety with formula —C(O)OR, where R is selected from the group consisting of alkyl, cycloalkyl, aryl, heteroaryl and heteroalicyclic.

The term heterocycle or heterocyclic group, also denoted by “Het” or “heterocyclyl”, can be a stable, saturated, partially unsaturated, or aromatic 5- or 6-membered heterocyclic group. The heterocyclic ring consists of carbon atoms and from one to three heteroatoms independently selected from the group consisting of nitrogen, oxygen, and sulfur. A heteroaryl group is heterocycle that is aromatic, such as pyridine. The heterocyclic group can be optionally substituted with one to four substituents independently selected from halogen, alkyl, aryl, hydroxy, alkoxy, haloalkyl, nitro, amino, acylamino, monoalkylamino, dialkylamino, alkylthio, alkylsulfinyl and alkylsulfonyl or, when the heterocyclyl group is an aromatic nitrogen-containing heterocyclic group, the nitrogen atom can carry an oxide group. Examples of such heterocyclic groups are imidazolyl, imidazolinyl, thiazolinyl, pyridyl, indolyl, furyl, pyrimidinyl, morpholinyl, pyridazinyl, pyrazinyl, triazinyl and triazolyl. Two or more heterocycles may be fused to form polyheterocycles such as, for example, azaindole or purine.

The terms “heteroaryl” or, alternatively, “heteroaromatic” refers to an aryl group that includes one or more ring heteroatoms selected from nitrogen, oxygen and sulfur. An N-containing “heteroaromatic” or “heteroaryl” moiety refers to an aromatic group in which at least one of the skeletal atoms of the ring is a nitrogen atom. In certain embodiments, heteroaryl groups are monocyclic or polycyclic.

The term“heterocycloalkyl” refers to a cyclic non-aromatic compound that is saturated or partially unsaturated and may contain one or more carbonyl(C═O) functional groups in the ring.

The term “tryptophan derivative” or “tryptophan analog” refers to the amino acid tryptophan that is substituted with one or more substituents other than hydrogen on one or more of its aromatic rings, and such that the substituents correspond to the definitions provided for A, B, C, D, A′, B′, C′, and D′, and/or a tryptophan that is substituted at the ring positions with C or N as defined for E, F, G, M, E′, F′, G′, and M′ above.

The term “substituted” means a substituent or function group or groups, such as those described for A, B, C, and D, are attached to carbon of the main hydrocarbon scaffold in place of hydrogen.

The term “leaving group” (LG) as used in the specification is readily understood by those skilled in the art. Generally, a leaving group is any group or atom that enhances the electrophilicity of the atom to which it is attached for easy displacement by a nucleophilic group or atom. Examples of preferred leaving groups are triflate (—OSO₂CF₃) mesylate, tosylate, imidate, chloride, bromide, and iodide.

Under certain circumstances it is at least desired and often required to protect the nitrogen (N) of intermediates during the synthesis of the compounds of formulae (I) with suitable “protecting groups” which are known. Introduction and removal of such nitrogen protecting groups are well-known to those skilled in the art.

In this regard, the term “—NH protective groups” and “protecting group” when used in a similar context, and as used in the specification and claims, refers to sub-class of amino protecting groups that are commonly employed to block or protect the —NH functionality while reacting other functional groups on the compound. The species of protecting group employed in carrying out the method of the present disclosure is not critical so long as the derivatized —NH group is stable to the condition(s) of subsequent reaction(s) and can be removed at the appropriate point without disrupting the remainder of the molecule. T. W. Greene and P. Wuts, Protective Groups in Organic Synthesis, Chapter 7, pages 385-394 and 397-403, provide a list of commonly employed protecting groups for indoles and maleimides. Preferred indole protecting groups are trimethylsilylethoxymethyl, benzyl, tosyl, carbamate, amide, alkyl or aryl sulfonamide, while maleimide protecting groups include alkoxy, benzyl, dialkoxybenzyl, benzyloxyalkyl or allyl. The related term “protected —NH” defines a group substituted with an —NH protecting group.

In certain circumstances there may also be a need to protect hydroxy groups and amino groups during the synthetic processes of the present disclosure. Those skilled in the art are familiar with such “hydroxy protecting groups” and such “amino protecting groups,” The term “hydroxy protecting group” refers to one of the ether or ester derivatives of the hydroxy group commonly employed to block or protect the hydroxy group while reactions are carried out on other functional groups on a compound. The species of hydroxy protecting group employed is not critical so long as the derivatized hydroxy group is stable to the condition of subsequent reaction(s) and can be removed at the appropriate point without disrupting the remainder of the molecule. Preferred hydroxy protecting groups are tertbutyldiphenylsilyloxy (TBDPS), tert-butyldimethylsilyloxy (TBDMS), triphenylmethyl (trityl), mono- or dimethoxytrityl, or an alkyl or aryl ester.

The term “amine protecting group” refers to substituents of an amino group commonly employed to block or protect the amino functionality while reacting other functional groups on the compound. The species of amino-protecting group employed in carrying out the method of the present disclosure is not critical so long as the derivatized amino group is stable to the condition(s) of subsequent reaction(s) and can be removed at the appropriate point without disrupting the remainder of the molecule. Preferred amino-protecting groups are t-butoxycarbonyl, phthalimide, a cyclic alkyl, and benzyloxycarbonyl.

The term “activated maleimide” as used in the specification refers to a 3,4-disubstituted maleimide (pyrrolyl-2,5-dione) or 2,3,4-trisubstituted maleimide, substituted with at least one leaving group that facilitates reaction with a reagent and especially with an optionally N-substituted organometallic-3-indole.

The term “indolylmaleimide” embraces a genus of compounds having as their root structure a 3-(indol-3-yl)-pyrrolyl-2,5-dione and includes the subgenus of“bisindolylmaleimides” having as their root structure a 3,4-(indol-3-yl)-pyrrolyl-2,5-dione, wherein the indol-3-yl moiety or moieties is/am optionally N-substituted, may optionally be substituted on the fined 6-membered aromatic ring of the indolyl moiety and may optionally be substituted at position 2 of the indol-3-yl moiety or moieties. Also included are those bisindolylmaleimides wherein the N-substituents of the indolyls are linked together through a bridging moiety as described for Q and R above in Formula (I) and for Formula (II). The prior art describes a range of such optionally substituted indolylmaleimides.

The term “indolocarbazole” refers to an alkaloid compound containing two indole rings derived from tryptophan and a fused maleimide or lactam functionality, and derivatives thereof. The most frequently isolated natural indolocarbazoles are indolo(2,3-a)carbazoles and the most common subgroup are the indolo(2,3-a)pyrrole(3,4-c)carbazoles.

As described herein, compounds of this disclosure may contain “optionally substituted” moieties. In general, the term “substituted,” whether preceded by the term “optionally” or not, means that one or more hydrogens of the designated moiety are replaced with a suitable substituent Unless otherwise indicated, an “optionally substituted” group may have a suitable substituent at each substitutable position of the group, and when more than one position in any given structure may be substituted with more than one substituent selected from a specified group, the substituent may be either the same or different at every position. The term “stable,” as used herein, refers to compounds that ae not substantially altered when subjected to conditions to allow for their production, detection, and, in certain embodiments, their recovery, purification, and use for one or more of the purposes disclosed herein.

As used herein, the term “solubilizing group” refers to a chemical moiety that promotes the solubility of a compound to which it is attached. Suitable solubilizing groups include, for example, saturated heterocyclic rings, such as morpholino, piperazinyl, and piperadinyl, and amino groups, such as dimethyl amino and methoxypropylamino.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of this disclosure. Unless otherwise stated, all tautomeric forms of the compounds of this disclosure are within the scope of this disclosure.

The term “isotopic variant” refers to a compound that contains an unnatural proportion of an isotope at one or more of the atoms that constitute such a compound. In certain embodiments, an “isotopic variant” of a compound is in an unstable form, that is, radioactive. Such compounds are useful, for example, as analytical tools, as probes in biological assays, or as therapeutic agents in accordance with the present disclosure.

The term “solvate” refers to a complex or aggregate formed by one or more molecules of a solute, e.g., a compound provided herein, and one or more molecules of a solvent, which present in a stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The term “naturally occurring” or “natural” or “native” when used in connection with naturally occurring biological materials such as nucleic acid molecules, amino acids, polypeptides, small molecule natural products, host cells, and the like, refers to materials that are found in or isolated directly from Nature and are not changed or manipulated by humans. Similarly, “non-naturally occurring” or “non-natural” or “unnatural” or “non-native” refers to a material that is not known to exist or not found in Nature or that has been structurally modified or synthesized by humans.

The term “semi-synthesis” refers to modifying a natural material synthetically to create a new variant, derivative, or analog of the original natural material. The terms “derivative” or “analog” refer to a structural variant of compound that derives from a natural or nan-natural material.

The terms “optically active” and “enantiomerically active” refer to a collection of molecules, which has an enantiomeric excess of no less than about 50%, no less than about 70%, no less than about %, no less than about 90%, no less than about 91%, no less than about 92%, no less than about 93%, no less than about 94%, no less than about 95%, no less than about 96%, no less than about 97%, no less than about 9%, no less than about 99%, no less than about 99.5%, or no less than about 99.8%. In certain embodiments, the compound comprises about 95% or more of one enantiomer and about 5% or less of the other enantiomer based on the total weight of the racemate in question. In describing an optically active compound, the prefixes R and S are used to denote the absolute configuration of the molecule about its chiral center(s). The symbols (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The phrase “a stereoisomer, enantiomer, mixture of enantiomers, mixture of diastereomers, or isotopic variant thereof; or a pharmaceutically acceptable salt, solvate, hydrate, or prodrug thereof,” has the same meaning as the phrase “a stereoisomer, enantiomer, mixture of enantiomers, mixture of diastereomers, or isotopic variant of the compound referenced therein; a pharmaceutically acceptable salt, solvate, hydrate, or prodrug of the compound referenced therein; or a pharmaceutically acceptable salt, solvate, hydrate, or prodrug of a stereoisomer, enantiomer, mixture of enantiomers, mixture of diastereomers, or isotopic variant of the compound referenced therein.”

The term “about” or“approximately” means an acceptable error for a particular value as determined by one of ordinary skill in the art, which depends in pat on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a disorder, disease, or condition. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a disorder, disease, or condition.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The term “patient”, as used herein, means an animal, preferably a mammal, and most preferably a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subjects risk of acquiring a disorder, disease, or condition.

The term “therapeutically effective amount” are meant to include the amount of a compound that, when administered, is sufficient to prevent development of or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers an amount, concentration, or dosage of a compound that is required for 50% inhibition of a maximal response in an assay that measures such response. The term “CC₅₀” refers an amount, concentration, or dosage of a compound that results in 50% reduction of the viability of a host. In certain embodiments, the CCA of a compound is the amount, concentration, or dosage of the compound that is required to reduce the viability of cells treated with the compound by 50%, in comparison with cells untreated with the compound. The term “K_{d}” refers to the equilibrium dissociation constant for a ligand and a protein, which is measured to assess the binding strength that a small molecule ligand (such as a small molecule drug) has for a protein, such as a kinase. The dissociation constant, K_{d}, is commonly used to describe the affinity between a ligand and a protein; i.e., how tightly a ligand binds to a particular protein, and is the inverse of the association constant. Ligand-protein affinities are influenced by non-covalent intermolecular interactions between the two molecules such as hydrogen bonding, electrostatic interactions, hydrophobic and van der Waals forces. The analogous term “Kᵢ” is the inhibitor constant or inhibition constant, which is the equilibrium dissociation constant for an enzyme inhibitor, and provides an indication of the potency of an inhibitor.

As used herein, the phrase “biologically active” refers to a characteristic of any substance that has activity in a biological system and/or organism. For instance, a substance that, when administered to an organism, has a biological effect on that organism is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, the term “efiective amount” is an amount, which when administered systemically, is sufficient to effect beneficial or desired results, such as beneficial or desired clinical results, or other desired effects that lead to an improvement of the disease condition. An effective amount is also an amount that produces a prophylactic effect. e.g., an amount that delays, reduces, or eliminates the appearance of a pathological or undesired condition associated with an autoimmune disease or cancer. An effective amount is optionally administered in one or more administrations. In terms of treatment, an “effective amount” of a composition described herein is an amount that is sufficient to palliate, alleviate, ameliorate, stabilize, reverse or slow the progression of an autoimmune disease or cancer.

As used herein, the term “inhibitor” refers to a molecule which is capable of inhibiting (including partially inhibiting or allosteric inhibition) one or more of the biological activities of a target molecule, e.g., a PIM kinase. Inhibitors, for example, act by reducing or suppressing the activity of a target molecule and/or reducing or suppressing signal transduction. In some embodiments a PIM inhibitor described herein causes substantially complete inhibition of all three PIM kinases. In some embodiments, a PIM inhibitor described herein causes substantially complete inhibition of two PIM kinases, such as PIM1 and PIM3. In some embodiments, a PIM inhibitor described herein causes substantially complete inhibition of one PIM kinases, such as PIM3. In some embodiments, the phrase “partial inhibitor” refers to a molecule which can induce a partial response for example, by partially reducing or suppressing the activity of a target molecule and/or partially reducing or suppressing signal transduction. In some instances, a partial inhibitor mimics the spatial arrangement, electronic properties, or some other physiochemical and/or biological property of the inhibitor. In some instances, in the presence of elevated levels of an inhibitor; a partial inhibitor competes with the inhibitor for occupancy of the target molecule and provides a reduction in efficacy, relative to the inhibitor alone.

In some embodiments, a PIM inhibitor described herein is a partial inhibitor of PIM kinases. In some embodiments, a PIM inhibitor described herein is an allosteric modulator of PIM kinases. In some embodiments, a PIM inhibitor binds to the kinase domain of PIM kinases. In some embodiments, the PIM inhibitor described herein blocks the ATP binding site of PIM. In some embodiments, a PIM inhibitor is a “Type II” kinase inhibitor. In some embodiments a PIM inhibitor stabilizes the PIM kinases in their inactive conformation or state. In some embodiments, a PIM inhibitor stabilizes the “DFG-out” conformation of PIM kinases.

In some embodiments, PIM inhibitors reduce, abolish, and/or remove the binding between PIM and at least one of its natural binding partners (e.g. pro-apoptotic Bcl-2-associated death promoter protein (BAD), the ribosomal protein 4E-BP1, and transcription factor c-Myc). In some instances, binding between PIM and at least one of its natural partners is stronger in the absence of a PIM inhibitor (by e.g., 90%, 80%, 70%, 60%, 50%, 40%, 30° A or 20%) than in the presence of a PIM inhibitor. Alternatively or additionally, PIM inhibitors inhibit the phosphotransferase activity of PIM kinases, e.g., by binding directly to the catalytic site or by altering the conformation of PIM such that the catalytic site becomes inaccessible to substrates. In some embodiments, PIM inhibitors inhibit the ability of PIM kinases to phosphorylate at least one of its target substrates, e.g., transcription factors STAT3 and STAT5 (Signal Transducers and Activators of Transcription), c-Myc, FoxO1a, and FoxD3a, the cell cycle regulators p27, Cdc25A, and Cdc25C, or itself. PIM inhibitors include inorganic and/or organic compounds.

In some embodiments, PIM inhibitors described herein decrease signal transduction induced by mitogenic growth factors such as interleukins and interferons binding to cytokine receptors. In some embodiments, PIM inhibitors described herein decrease phosphorylation of the pro-apoptotic BAD, thus enabling cell apoptosis. In some embodiments, PIM inhibitors described herein decrease cellular levels of transcription factor protein c-Myc. In some embodiments, PIM inhibitors described herein decrease cellular levels of peroxisome proliferator-activated receptor gamma coactivator 1α (POC-1α), an enzyme capable of regulating glycolysis and mitochondrial biogenesis. In some embodiments, PIM inhibitors described herein decrease phosphorylation and activation of transcription factors STAT3, Myb, FoxO1a, and FoxO3a. In some embodiments, PIM inhibitors described herein increase glucose tolerance and insulin sensitivity. In some embodiments, PIM inhibitors described herein reduce levels of VEGF and angiogenesis by phosphorylating STAT3. In some embodiments, PIM inhibitors described herein decrease cell proliferation of pancreatic cancer cells. In some embodiments, PIM inhibitors described herein decrease cell proliferation of gastric cancer cells. In some embodiments, PIM inhibitors described herein decrease cell proliferation of colorectal cancer cells. In some embodiments, PIM inhibitors described herein decrease cell proliferation of prostatic cancer cells. In some embodiments, PIM inhibitors described herein decrease cell proliferation of gallbladder cancer cells. In some embodiments, PIM inhibitors described herein decrease cell proliferation of nasopharyngeal caner cells. In some embodiments, PIM inhibitors described herein decrease cell proliferation of hepatic cancer cells

In some embodiments, a PIM inhibitor suitable for the methods described herein is a direct PIM inhibitor. In some embodiments, a PIM inhibitor suitable for the methods described herein is an indirect PIM inhibitor. In some embodiments a PIM inhibitor suitable for the methods herein decreases PIM activity relative to a basal level of PIM activity by about 1.1 fold to about 1000 fold, e.g., to about 1.2 fold, 1.5 fold, 1.6 fold, 1.7 fold, 2.0 fold, 3.0 fold, 5.0 fold, 6.0 fold, 7.0 fold, 8.5 fold, 9.7 fold, 10 fold, 12 fold, 14 fold, 15 fold, 20 fold, 30 fold, 40 fold, 50 fold, 60 fold, 70 fold, 80 fold, 90 fold, 95 fold, 100 fold, 200 fold, 300 fold, 400 fold, 500 fold, 600 fold, 700 fold, 800 fold, 900 fold, 1000 fold, or by any other amount from about 1.1 fold to about 1000 fold relative to basal PIM3 activity. In some embodiments, the PIM inhibitor is a reversible PIM inhibitor. In other embodiments, the PIM inhibitor is an irreversible PIM inhibitor. Direct PIM inhibitors are optionally used for the manufacture of a medicament for treating a malignant or cancerous disease.

In some embodiments, a PIM inhibitor used for the methods described herein has an in vitro IC₅₀, defined as inhibitory concentration where 50% of the activity of one or more PIM kinases is remaining after contacting a PIM inhibitor with PIM kinase, or dissociation constant (K_{d}), or inhibitory constant (K) of less than 100 μM (e.g., less than 10 μM, less than 5 μM, less than 4 μM, less than 3 μM less than 1 μM, less than 0.8 μM, less than 0.6 μM, less than 0.5 μM, less than 0.4 μM, less than 0.3 μM, less than less than 0.2 μM. less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092 μM, less than 0.0090 μM, less than 0.0010 μM, or less than 0.00010 μM).

As used herein, “expression” of a nucleic acid sequence refers to one or more of the following events (1) production of an RNA template from a DNA sequence (e.g., by transcription of DNA into messenger RNA); (2) processing of an RNA transcript (e.g. by splicing, editing. 5′ cap formation, and/or 3′ end formation); (3) translation of an RNA into a polypeptide or protein; (4) post-translational modification of a polypeptide or protein.

As used herein the term “PIM polypeptide” or “PIM protein” or “PIM” or “PIM kinase” refers to a protein that belongs in the serine/threonine family of human kinases. A representative example of PIM1 amino acid sequences includes, but is not limited to, human PIM (GenBank Accession Number P11309). Human PIM1 also has two truncated isoforms of 34 kDa and 44 kDa that have been identified and PIM1 homologues exist throughout the animal kingdom. A representative example of PIM2 amino acid sequences includes, but is not limited to, human PIM2 (GenBank Accession Number Q9P1W9). Human PIM2 also has three truncated isoforms of 34 kDa, 37 kDa, and 40 kDa that have been identified and PIM2 homologues exist throughout the animal kingdom. PIM3 amino acid sequences include, but are not limited to, human PIM3 (GenBank Accession Number Q86V86). Human PIM3 also has numerous truncated isoforms that have been identified and PIM3 homologues exist throughout the animal kingdom.

In some embodiments, a PIM1 polypeptide comprises an amino acid sequence that is at least 60% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Number P11309. In some embodiments, a PIM2 polypeptide comprises an amino acid sequence that is at least 60% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Number Q9P1W9. In some embodiments, a PIM3 polypeptide comprises an amino acid sequence that is at least 60% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Number Q86V86.

A representative example of human PIM1 genes encoding PIM1 proteins include, but are not limited to, human PIM1 (GenBank Accession Number 5292). In some embodiments, a human PIM1 gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Number 5292. A representative example of human PIM2 genes encoding PIM2 proteins include, but are not limited to, human PIM2 (GenBank Accession Number 11040). In some embodiments, a human PIM2 gene comprises a nucleotide sequence that is at least 70% to 100% identical, e.g., at least 75%, 80%, 856%, 87%, 88%, 90%, 91%, 92%, 94%, 95%, 96%, 97%, 98%, or any other percent from about 70% to about 100% identical to sequences of GenBank Accession Number 11040. A representative example of human PIM3 genes encoding PIM3 proteins include, but are not limited to, human PIM3 (GenBank Accession Number 415116). In some embodiments, a human PIM3 gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Number 415116.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence) The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In one embodiment the two sequences are the same length.

To determine percent homology between two sequences, the algorithm of Karlin, S. and Altschul, S. F., Proc. Natl. Acad. Sci. USA, 1990, 872264-2268, modified as in Karlin, S. and Altschul S. F., Proc. Nat. Acad. Sci. USA, 1993, 90:5873-5877 is used. Such an algorithm is incorporated into the NBLAST and BLAST programs of Altschul, S. F., et al., J. Mo. Biol., 1990, 215, 403-410. BLAST nucleotide searches are performed with the NBLAST program, score=100, wordlength=12 to obtain nucleotide sequences homologous to a nucleic acid molecules described or disclose herein. BLAST protein searches are performed with the BLAST program, score=50, wordlength=3. To obtain gapped alignments for comparison purposes, Gapped BLAST is utilized as described in Altschul, S. F., et al. Nucleic Acids Res., 1997, 25, 3399-3402. When utilizing BLAST and Gapped BLAST programs, the default parameters of the respective programs (e.g., BLAST and NBLAST) we used. See the website of the National Center for Biotechnology Information for further details (www.ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein PIM3 inhibitor described herein. In other embodiments, the altered amino acid sequence is at least 75% identical, e.g., 77%, 80%, 2% 85%, 8%, 90%, 92%, 95%, 97%, 98%, 99%, or 100% identical to the amino acid sequence of any protein PIM3 inhibitor described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the 20 common proteinogenic amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff, S., et al., Proc. Natl. Acad. Sci. USA, 1992, 89, 10915-10919). Accordingly, the BLOSUM62 substitution frequencies are used to define conservative amino acid substitutions that may be introduced into the amino acid sequences described or described herein. Although it is possible to design amino acid substitutions based solely upon chemical properties (as discussed above), the language “conservative amino acid substitution” preferably refers to a substitution represented by a BLOSUM62 value of greater than. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “PIM activity,” unless otherwise specified, includes, but is not limited to, at least one of PIM kinase protein-protein interactions, PIM phosphotransferase activity (intermolecular or intermolecular), translocation, etc of one or more PIM isoforms. As used herein, a “PIM inhibitor” refers to any molecule, compound, or composition that directly or indirectly decreases the PIM activity. In some embodiments, PIM inhibitors inhibit, decrease, and/or abolish the level of a PIM mRNA and/or protein or the half-life of PIM mRNA and/or protein, such inhibitors are referred to as “clearance agents”. In some embodiments, a PIM inhibitor is a PIM antagonist that inhibits, decreases, and/or abolishes an activity of PIM. In some embodiments, a PIM inhibitor also disrupts, inhibits, or abolishes the interaction between PIM and its natural binding partners (e.g., a substrate for PIM3 kinase, for BAD, or for c-Myc) or a protein that is a binding partner of PIM in a pathological condition, as measured using standard methods.

In some embodiments, PIM3 inhibitors reduce, abolish, and/or remove the binding between PIM and at least one of its natural binding partners (e.g., BAD, AMPK, STAT3, c-Myc, Myb, FoxO1a, and FoxO3a, p21, p27, PGC-1α, eIF4B, Cdc25A, Cdc25C, or translationally controlled tumor protein TCTP/TPT1), In some instances, binding between PIM and at least one of its natural binding partners is stronger in the absence of a PIM inhibitor (by e.g., 90%, 80%, 70%, 60%, 50%, 40%, 30% or 20%) than in the presence of a PIM inhibitor. In some embodiments, PIM inhibitors prevent, reduce, or abolish binding between PIM and a protein that abnormally accumulates or aggregates in cells or tissue in a disease state. In some instances, binding between PIM and at least one of the proteins that aggregates or accumulates in a cell or tissue is stronger in the absence of a PIM inhibitor (by e.g., 90%, 80%, 70%, 60%, 50%, 40%, 30% or 20%) than in the presence of an inhibitor. An “individual” or an “individual,” is a mammal. In some embodiments, an individual is an animal, for example, a rat, a mouse, a dog or a monkey. In some embodiments, an individual is a human patient. In some embodiments, an individual suffers from cancer or is suspected to be suffering from cancer or is genetically pre-disposed to cancer. In some embodiments, a pharmacological composition comprising a PIM inhibitor is “administered peripherally” or “peripherally administered.” As used herein, these terms refer to any form of administration of an agent, e.g., a therapeutic agent, to an individual that is not direct administration to the central nervous system, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation transbuccal, intranasal, rectal, oral, parenteral, sublingual, or transnasal. In some embodiments, a PIM3 inhibitor is administered by an intracerebral route.

The terms “polypeptide,” and “protein” are used interchangeably herein to refer to a polymer of amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues an linked by covalent peptide bonds.

The term “amino acid” refers to naturally occurring and non-naturally occurring amino acids, as well as amino acid analogs and amino acid mimetics that function in a manner similar to the naturally occurring amino acids. The term “nucleic acid” refers to deoxyribonucleotides, deoxyribonucleosides, ribonucleosides, or ribonucleotides and polymers thereof in either single- or double-stranded form. Unless specifically limited, the term encompasses nuclei: acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer, M A., et al., Nucleic Acid Res, 1991, 19, 5091-1585; Ohtsuka, E. et al., J. Biol. Chem., 1985, 260, 2605-2608; and Rossolini, G. M., et al., Mol. Cell. Probes, 1994, 8, 91-98).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc.) in a sample. In another example, a polypeptide is purified if it is substantially removed from or concentrated in its natural environment. Methods for purification and isolation of nucleic acids and proteins are documented methodologies.

The term “antibody” describes an immunoglobulin whether natural or party or wholly synthetically produced. The term also covers any polypeptide or protein having a binding domain which is, or is homologous to, an antigen binding domain. CDR grafted antibodies are also contemplated by this term. The term antibody as used herein will also be understood to mean one or more fragments of an antibody that retain the ability to specifically bind to an antigen, (See generally: Holliger, P. et al., Nature Biotech. 2005, 23 (9), 1126-1129).

By “assaying” is meant the creation of experimental conditions and the gathering of data regarding a particular result of the exposure to specific experimental conditions. For example, enzymes can be assayed based on their ability to act upon a detectable substrate. A compound can be assayed based on its ability to bind to a particular target molecule or molecules.

As used herein, the term “modulating” or “modulate” refers to an effect of altering a biological activity (i.e. increasing or decreasing the activity), especially a biological activity associated with a particular biomolecule such as a protein kinase. For example, an inhibitor of a particular biomolecule modulates the activity of that biomolecule, e.g., an enzyme, by decreasing the activity of the biomolecule, such as an enzyme. Such activity is typically indicated in terms of an inhibitory concentration (IC₅₀) of the compound for an inhibitor with respect to, for example, an enzyme.

In the context of the use, testing, or screening of compounds that an or may be modulators, the term “contacting” means that the compound(s) are caused to be in sufficient proximity to a particular molecule, complex, cell, tissue, in an organism, or other specified material that potential binding interactions and/or chemical reaction between the compound and other specified material can occur.

### Kinase Activity Assay,

A number of different assays for kinase activity can be utilized for assaying for active modulators md/or determining specificity of a modulator for a particular kinase or group of kinases. In addition to the assays mentioned in the Examples below, one of ordinary skill in the art will know of other assays that can be utilized and can modify an assay for a particular application. For example, numerous papers concerning kinases describe assays that can be used. Additional alternative assays can employ binding determinations. For example, this sort of assay can be formatted either in a fluorescence resonance energy transfer (FRET) format, or using an AlphaScreen (amplified luminescent proximity homogeneous assay) format by varying the donor and acceptor reagents that are attached to streptavidin or the phosphor-specific antibody.

As used herein, the term “biopharmaceutical properties” refers to the pharmacokinetic action of a compound or complex of the present disclosure, including the dissolution, absorption and distribution of the compound on administration to a subject. As such, certain solid forms of compounds of this disclosure, such as amorphous complexes of compounds of this disclosure, are intended to provide improved dissolution and absorption of the active compound, which is typically reflected in improved Cmax, (the maximum achieved concentration in the plasma after administration of the drug) and improved AUC (i.e. are under the curve of drug plasma concentration vs. time after administration of the drug).

### Alternative Compound Forms or Derivatives

Compounds contemplated herein are described with reference to both generic formulae and specific compounds. Alternative forms or derivatives, include, for example, (a) prodrugs, and active metabolites (b) tautomers, isomers (including stereoisomers and regioisomers), and racemic mixtures (c) pharmaceutically acceptable salts and (d) solid forms, including different crystal forms, polymorphic or amorphous solids, including hydrates and solvates thereof, and other forms.

### (a) Prodrugs and Metabolites

In addition to the present formulae and compounds described herein, this disclosure also includes prodrugs (generally pharmaceutically acceptable prodrugs), active metabolic derivatives (active metabolites), and their pharmaceutically acceptable salts.

Prodrugs are compounds or pharmaceutically acceptable salts thereof which, when metabolized under physiological conditions or when converted by solvolysis, yield the desired active compound. Some prodrugs are activated enzymatically to yield the active compound, or a compound may undergo further chemical reaction to yield the active compound. Prodrugs may proceed from prodrug form to active form in a single step or may have one or more intermediate forms which may themselves have activity or may be inactive.

As described in The Practice of Medicinal Chemistry, Ch. 31-32 (Ed. Wermuth, Academic Press, San Diego, Calif., 2001), prodrugs can be conceptually divided into two non-exclusive categories, bioprecursor prodrugs and carrier pro-drugs. Generally, bioprecursor prodrugs are compounds that are inactive or have low activity compared to the corresponding active drug compound that contain one or more protective groups and are converted to an active form by metabolism or solvolysis. Both the active drug form and any released metabolic products should have acceptably low toxicity. Typically, the formation of active drug compound involves a metabolic process or reaction.

### (b) Tautomers, Stereoisomers, and Regioisomers

It is understood that some compounds may exhibit tautomerism. In such cases, the formulae provided herein expressly depict only one of the possible tautomeric forms. It is therefore to be understood that the formulae provided herein are intended to represent any tautomeric form of the depicted compounds and are not to be limited merely to the specific tautomeric form depicted by the drawings of the formulae. Likewise, some of the compounds according to the present disclosure may exist as stereoisomers, i.e. having the same atomic connectivity of covalently bonded atoms yet differing in the spatial orientation of the atoms. Unless specified to the contrary, all such stereoisomeric forms are included within the formulae provided herein.

In some embodiments, a chiral compound of the present disclosure is in a form that contains at least 80% of a single isomer (60% enantiomeric excess (“e.e”) or diastereomeric excess (“d.e.”)), or at least 85% (70% e.e, or d.e.), 90%(80% e.e. or d.e.), 95%(90% e.e. or d.e.), 97.5%(95% e.e. or d.e.), or 99%(98% e.e. or d.e.). As generally understood by those skilled in the art, an optically pure compound having one chiral center is one that consists essentially of one of the two possible enantiomers (i.e., is enantiomerically pure), and an optically pure compound having more than one chiral center is one that is both diastereomerically pure and enantiomerically pure. In some embodiments, the compound is present in optically pure form, such optically pure form being prepared and/or isolated by methods known in the art (e.g. by recrystallization techniques, chiral synthetic techniques (including synthesis from optically pure starting materials), and chromatographic separation using a chiral column.

### (c) Pharmaceutically Acceptable Salts

Unless specified to the contrary, specification of a compound herein includes pharmaceutically acceptable salts of such compound.

As used herein, the term “pharmaceutically acceptable salt” refers to those salts which are, within the scope of sound medical judgment, suitable for use in contact with the tissues of humans and lower animals without undue toxicity, irritation, allergic response and the like, and re commensurate with a reasonable benefit/risk ratio. Pharmaceutically acceptable salts are well known in the art. For example, pharmaceutically acceptable salts are described in S. M. Berge et al., J. Pharmaceutical Sciences, 1977, 66, 1-19, which is incorporated herein by reference.

### (d) Other Compound Forms

In the case of agents that are solids, it is understood by those skilled in the art that the compounds and salts may exist in different crystal or polymorphic forms, or may be formulated as co-crystals, or may be in an amorphous form, or may be any combination thereof (e.g. partially crystalline, partially amorphous, or mixtures of polymorphs) all of which are intended to be within the scope of the present disclosure and specified formulae.

Pharmaceutically Acceptable Compositions

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3^{rd} ed., Ash and Ash Eds.; Gower Publishing Company: 2007; Pharmaceutical Preformulation and Formulation, 2nd ed.; Gibson Ed.; CRC Press LLC: Boca Raton, Fla., 2009. The term “pharmaceutically acceptable carrier, adjuvant, or vehicle” refers to a non-toxic carrier, adjuvant, or vehicle that does not destroy the pharmacological activity of the compound with which it is formulated. According to another embodiment, this disclosure provides a composition comprising a compound of this disclosure or a pharmaceutically acceptable derivative thereof and a pharmaceutically acceptable carrier, adjuvant, or vehicle.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

As used herein, the term “inhibitorily active metabolite or residue thereof” means that a metabolite or residue thereof is also an inhibitor of PIM3 or a mutant thereof.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

Preferably, the compositions are administered orally, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. Pharmaceutically acceptable compositions of this disclosure may be orally administered in any orally acceptable dosage form including, but not limited to, capsules, tablets aqueous suspensions or solutions.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. As used herein, the term “inhibitor” is defined as a compound that binds to and/or inhibits a target protein kinase with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

A compound of the present disclosure may be tethered to a detectable moiety. One of ordinary skill in the art will recognize that a detectable moiety may be attached to a provided compound via a suitable substituent. As used herein, the term “suitable substituent” refers to a moiety that is capable of covalent attachment to a detectable moiety.

As used herein, the term “detectable moiety” is used interchangeably with the term “label” and relates to a moiety capable of being detected, e.g., primary labels and secondary label. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The terms “measurable affinity” and “measurably inhibit,” as used herein, means a measurable change in a protein kinase activity between a sample comprising a compound of the present disclosure, or composition thereof, and protein, and an equivalent sample comprising the protein kinase, in the absence of said compound, or composition thereof.

As used herein, the terms “M-mediated,” disorders or conditions as used herein means any disease or other deleterious condition in which PIM kinase, or a mutant thereof, are known to play a role. Accordingly, another embodiment of the present disclosure relates to treating or lessening the severity of one or more diseases in which one or more of the PIM kinases or a mutant thereof, are known to play a role. Specifically, the present disclosure relates to a method of treating or lessening the severity of a disease or condition selected from a proliferative disorder, wherein said method comprises administering to a patient in need thereof a compound or composition according to the present disclosure.

In some embodiments, the present disclosure provides a method for treating or lessening the severity of one or more disorders selected from the various forms of cancer. In some embodiments, the cancer is associated with a solid tumor. In certain embodiments, the cancer is breast cancer, pancreatic cancer, hepatocellular cancer, prostate cancer, gastric cancer, glioblastoma, lung cancer, cancer of the head and neck, colorectal cancer, bladder cancer, or non-small cell lung cancer. In some instances, the present disclosure provides a method for treating or lessening the severity of one or more disorders selected from squamous cell carcinoma, salivary gland carcinoma, ovarian carcinoma, or pancreatic cancer. In other embodiments, the cancer is associated with a soluble tumor, such as a leukemia, lymphoma or myeloma.

In some embodiments, the present disclosure provides a method for treating or lessening the severity of one or more immunological or hypersensitivity disorders, such as asthma, allergy, transplant rejection, graft versus host disease, and autoimmune diseases such as rheumatoid arthritis, amyotrophic lateral sclerosis, and multiple sclerosis, as well as in solid and hematologic malignancies such as leukemias, lymphomas, and myelomas, wherein said method comprises administering to a patient in need thereof a composition according to the present disclosure. Depending upon the particular condition, or disease, to be treated, additional therapeutic agents, which are normally administered to treat that condition, may also be present in the compositions of this disclosure. As used herein, additional therapeutic agents that are normally administered to treat a particular disease, or condition, are known as “appropriate for the disease, or condition, being treated.” For example, compounds of the present disclosure, or a pharmaceutically acceptable composition thereof, are administered in combination with chemotherapeutic agents to treat proliferative diseases and cancer. Examples of known chemotherapeutic agents include, but are not limited to, Adriamycin, dexamethasone, vincristine, cyclophosphamide, fluorouracil, topotecan, taxol, Interferons, platinum derivatives, taxane (e.g., paclitaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, emetine, trimetrexate, metoprine, cyclosporine, daunorubicin, teniposide, amphotericin, alkylating agents (e.g., chlorambucil), 5-fluoruracil, camptothecin, cisplatin, metronidazole, and GLEEVEC™ among others. In other embodiments, a compound of the present disclosure is administered in combination with a biologic agent, such as AVASTIN™ or VECTIBIX™. In certain embodiments, compounds of the present disclosure, or a pharmaceutically acceptable composition thereof, are administered in combination with an antiproliferative or chemotherapeutic agent.

As used herein, the term “combination,” “combined,” and related terms refers to the simultaneous or sequential administration of therapeutic agents in accordance with this disclosure. For example, a compound of the present disclosure may be administered with another therapeutic agent simultaneously or sequentially in separate unit dosage forms or together in a single unit dosage form. Accordingly, the present disclosure provides a single unit dosage form comprising a compound of Formulas (I)-(IX), an additional therapeutic agent, and a pharmaceutically acceptable carrier, adjuvant, or vehicle. The amount of both, an inventive compound and additions therapeutic agent (in those compositions which comprise an additional therapeutic agent as described above) that may be combined with the carrier materials to produce a single dosage form will vary depending upon the host treated and the particular mode of administration. Preferably, compositions of this disclosure should be formulated so that a dosage of between 0.01-100 mg/kg body weight/day of an inventive compound can be administered.

In those compositions which comprise an additional therapeutic agent, that additional therapeutic agent and the compound of this disclosure may act synergistically. Therefore, the amount of additional therapeutic agent in such compositions will be less than that required in a monotherapy utilizing only that therapeutic agent. In suck compositions a dosage of between 0.01-1,000 mg/kg body weight/day of the additional therapeutic agent can be administered. The amount of additional therapeutic agent present in the compositions of this disclosure will be no more than the amount that would normally be administered in a composition comprising that therapeutic agent as the only agent. Preferably the amount of additional therapeutic agent in the presently disclosed compositions will range from about 50% to 100% of the amount normally present in a composition comprising that agent as the only therapeutically active agent.

The compounds of this disclosure, or pharmaceutical compositions thereof, may also be incorporated into compositions for coating an implantable medical device, such as prostheses, artificial valves, vascular grafts, stents and catheters.

Drug resistance is emerging as a significant challenge for targeted therapies. For example, drug resistance has been reported for Gleevec™ and IRESSA™, as well as several other kinase inhibitors in development. Drug resistance, for example, has been reported for inhibitors cKit and EGPR kinases used for cancer treatment. It has been reported that irreversible inhibitors may be effective against drug resistant forms of protein kinases (See: Kwak, E. L, et al., Proc. Nat. Aced. Sci. USA, 2005, 102, 7665-7670). Compounds of the present disclosure may be effective inhibitors of drug resistant forms of protein kinases.

As used herein, the term “clinical drug resistance” refers to the loss of susceptibility of a drug target to drug treatment as a consequence of mutations in the drug target. As used herein, the term “resistance” refers to changes in the wild-type nucleic acid sequence coding a target protein or its promoter, and/or the protein sequence of the target, which changes decrease or abolish the inhibitory effect of the inhibitor on the target protein. For example, PIM3 inhibitor resistance also may involve expression of another protein kinase, such as PIM1, which compensates of the loss of PIM3 kinase activity. Examples of kinases that are inhibited by the compounds and compositions described herein and against which the methods described herein are useful against PIM kinases, or mutants thereof.

The activity of a compound utilized in this disclosure as an inhibitor of a target kinase, in particular the PIM kinases and preferably PIM3, or a mutant thereof, may be assayed in vitro, in vivo or in a cell line. In vitro assays include more assays that determine inhibition of either the phosphorylation activity and/or the subsequent functional consequences, or ATPase activity of activated target kinase, or a mutant thereof. Alternate in vitro assays quantitate the ability of the inhibitor to bind to a target kinase, e.g., PIM3. Inhibitor binding may be measured by radiolabelling the inhibitor prior to binding, isolating the inhibitor/target kinase complex and determining the amount of radiolabel bound. Alternatively, inhibitor binding may be determined by running a competition experiment where new inhibitors are incubated with target kinase bound to known radioligands. Detailed conditions for assaying a compound utilized in this disclosure as an inhibitor of certain kinases, or a mutant thereof, are set forth in the Examples below.

Protein kinases are a class of enzymes that catalyze the transfer of a phosphate group from ATP or GTP to an acceptor amino acid residue (e.g., tyrosine, serine, and threonine) residue located on a protein substrate. Receptor kinases act to transmit signals from the outside of a cell to the inside by activating secondary messaging effectors via a phosphorylation event. A variety of cellular processes are promoted by these signals, including proliferation, carbohydrate utilization, protein synthesis, angiogenesis, cell growth, and cell survival.

As used herein, the terms “treatment,” “treat,” and “treating” refer to reversing, alleviating, delaying the onset of, or inhibiting the progress of a disease or disorder, or one or more symptoms thereof; as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are inhibitors of target PIM kinases and ae useful for treating one or more disorders associated with activity of PIM kinase. Thus, in certain embodiments, the present disclosure provides a method for treating PIM-mediated disorders comprising the step of administering to a patient in need thereof a compound of the present disclosure, or pharmaceutically acceptable composition thereof.

In some embodiments, compounds of this disclosure are optionally administered in combination with a PIM inhibitor clearance agent. In some embodiments, compounds of this disclosure are optionally administered in combination with a compound that directly or indirectly decreases the activation or activity of the upstream effectors of PIM. For example, in some embodiments a compound that inhibits the activity of Janus kinases (JAK1-3) is used in combination, thereby reducing the activation of PIM kinase. For example, use of the JAK inhibitor to facitinib could reduce phosphorylation and production of active STAT3 and STAT5 and thus decrease the expression, activity or activation of PIM3 (See: Hodge, J. A, et al., Clin. Exp. Rheumatol., 2016; 34, 318-328). In some embodiments, PIM3 activation is also decreased by small molecules that bind directly to STAT3 and STAT5. In some embodiments, PIM3 inhibitors are used in combination with agents that bind directly to BAD or prevent PIM kinases from phosphorylating serine residues in these downstream effectors (e.g, BAD Ser112).

In some embodiments, compounds of this disclosure are optionally administered in combination with a compound that decreases the level of PIM kinases, including a peptide, polypeptide, or small molecule that inhibits dephosphorylation of a downstream target of PIM, such that phosphorylation of the downstream target remains at a level that leads to downregulation of PIM levels. In some embodiments, PIM activity is reduced or inhibited via activator and/or inhibition of an upstream regulator and/or downstream target of PIM. In some embodiments, the protein expression of a PIM is downregulated. In some embodiments, the amount of PIM in a cell is decreased. In some embodiments a compound that decreases PIM3 protein levels in cells also decreases the activity of PIM kinases in the cells. In some embodiments a compound that decreases PIM protein levels does not decrease PIM activity in cells. In some embodiments a compound that increases PIM activity in cells decreases PIM protein levels in the cells.

Any combination of a PFM inhibitor and second therapeutic agent is compatible with any method described herein.

In addition, a PIM inhibitor is optionally used in combination with procedures that provide additional or synergistic benefit to the patient. By way of example only, patients are expected to find therapeutic and/or prophylactic benefit in the methods described herein, wherein pharmaceutical composition of a PIM inhibitor and/or combinations with other therapeutics am combined with genetic testing to determine whether that individual is a carrier of a mutant gene that is correlated with certain diseases or conditions.

A PIM inhibitor and additional therapies are optionally administered before, during or after the occurrence of a disease or condition, and the timing of administering the composition containing a PIM inhibitor varies in some embodiments. Thus, for example, the PIM inhibitor is used as a prophylactic and administered continuously to individual with a propensity to develop conditions or diseases in order to prevent the occurrence of a disease or condition.

### Pharmaceutical Compositions, Formulations, and Methods of Administration

Provided herein, in certain embodiments, are compositions comprising a therapeutically effective amount of any compound described herein (e.g., a compound of Formulas (I)-(IX)). Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams& Wilkins, 1999).

Provided herein are pharmaceutical compositions that include PIM inhibitors and a pharmaceutically acceptable diluent(s), excipient(s), or carrier(s). In addition, the PIM inhibitor is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances. A pharmaceutical composition, as used herein, refers to a mixture of a PIM inhibitor with other chemical components, such as carriers, stabilizers, diluents, dispersing agents, suspending agents, thickening agents, and/or excipients. The pharmaceutical composition facilitates administration of the PIM inhibitor to an organism. In practicing the methods treatment or use provided herein, therapeutically effective amounts of a PIM inhibitor are administered in a pharmaceutical composition to a mammal having a condition, disease, or disorder to be treated. Preferably, the mammal is a human. A therapeutically effective amount varies depending on the severity and stage of the condition, the age and relative health of an individual, the potency of the PIM inhibitor used and other factors. The PIM inhibitor is optionally used singly or in combination with one or more therapeutic agents as components of mixtures.

The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast smelt formulations, tablets, capsules, pills, delayed release formulations, extended release formulations, pulsatile release formulations, multi-particulate formulations, and mixed immediate and controlled release formulations. The pharmaceutical compositions will include at least one PIM inhibitor, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these PIM inhibitors having the same type of activity. “Carrier materials” include any commonly used excipients in pharmaceutics and should be selected on the basis of compatibility with compounds disclosed herein, such as, a PIM inhibitor, and the release profile properties of the desired dosage form.

Pharmaceutical preparations for oral use are optionally obtained by mixing one or more solid excipient with a PIM inhibitor, optionally grinding the resulting mixture, and processing the mixture of granules, after adding suitable auxiliaries, if desired, to obtain tablets or dragee cores. Suitable excipients include, for example, fillers such as sugars, including lactose sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose or others such as: polyvinylpyrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol By way of example, Example 20 describes an oral solid dosage formulation that is a tablet.

The pharmaceutical solid oral dosage forms including formulations described herein, which include a PIM inhibitor, are optionally further formulated to provide a controlled release of the PIM inhibitor. Controlled release refers to the release of the PIM inhibitor from a dosage form in which it is incorporated according to a desired profile over an extended period of time. Controlled release profiles include, for example, sustained release, prolonged release, pulsatile release, and delayed release profiles.

In other embodiments, the formulations described herein, which include a PIM inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include a PIM inhibitor, are optionally administered using a variety of pulsatile formulations that include, but are not limited to, those described in U.S. Pat. Nos. 5,011,692; 5,017,381, 5,229,135, and 5,840,329. Other pulsatile release dosage forms suitable for use with the present formulations include, but are not limited to, for example, U.S. Pat. Nos. 4,871,549; 5,260,068; 5,260,069; 5,508,040; 5,567,441; and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the PIM inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401; 6,667,048; and 6,960,563.

Suitable intranasal formulations include those described in, for example, U.S. Pat. Nos. 4,476,116 and 5,116,817 and amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the PIM inhibitor is optionally in a form as an aerosol, a mist or a powder. Pharmaceutical compositions described herein are conveniently delivered in the form of an aerosol spray presentation from pressurized packs or a nebulizer, with the use of a suitable propellant, e.g., difluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges o such as, by way of example only, gelatin for use in an inhaler or insufflator are formulated containing a powder mix of the PIM inhibitor and a suitable powder base such as lactose or starch.

Transdermal formulations of a PIM inhibitor are administered through the skin. The transdermal formulations described herein include at least three components: (1) a formulation of a PIM inhibitor; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing to material to enhance absorption and prevent the removal of the transdermal formulation from the skin. In other embodiments, the transdermal formulations described herein maintain a saturated or supersaturated state to promote diffusion into the skin. Formulations that include a PIM inhibitor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of

### Methods of Dosing and Treatment Regimens

The PIM3 inhibitor is optionally used in the preparation of medicaments for the prophylactic and/or therapeutic treatment of a disease or disorder that would benefit, at least in part, from amelioration of symptoms. In addition, a method for treating any of the diseases or conditions described herein in an individual in need of such treatment involves administration of pharmaceutical compositions containing at least one PIM3 inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said individual.

In the case wherein the patient's condition does not improve, upon the doctor's discretion the administration of the PIM3 inhibitor is optionally administered chronically, that is, for an extended period of time, including throughout the duration of the patients life in order to ameliorate or otherwise control or limit the symptoms of the patients disease or condition.

In the case wherein the patients status does improve, upon the doctor's discretion the administration of the PIM3 inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35% 40%, 45%, 50%, 55%, 60%, 65%, 70%, 75%, 80%, 85%, 90%, 95%, or 100%. Once improvement of the patients conditions has occurred, a maintenance dose is administered if necessary. Subsequently, the dosage or the frequency of administration, or both, is reduced, as a function of the symptoms, to a level at which the improved disease, disorder or condition is retained.

In some embodiments, patients require intermittent treatment on a long-term basis upon any recurrence of symptoms. In some embodiments, the pharmaceutical compositions described herein are in unit dosage forms suitable for single administration of precise dosages. In unit dosage form, the formulation is divided into unit doses containing appropriate quantities of one or more PIM inhibitors. In some embodiments, the unit dosage is in the form of a package containing discrete quantities of the formulation. Non-limiting examples are packaged tablets or capsules, and powders in vials or ampoules. In so embodiments, aqueous suspension compositions are packaged in single-dose non-reclosable containers. Alternatively, multiple-dose re-closable containers are used, in which case it is typical to include a preservative in the composition. By way of example only, formulations for parenteral injection are presented in unit dosage form, which include, but are not limited to ampoules, or in multi dose containers, with an added preservative. The daily dosages appropriate for the PIM3 inhibitor are from about 0.01 to about 2.5 mg/kg per body weight. An indicated daily dosage in the larger mammal, including, but not limited to, humans, is in the range from about 0.5 mg to about 1000 mg, conveniently administered in divided doses, including, but not limited to, up to four times a day or in extended release form. Suitable unit dosage forms for oral administration include from about 1 to about 500 mg active ingredient, from about 1 to about 250 mg of active ingredient, or from about 1 to about 100 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of the PIM inhibitor used, the disease or condition to be treated, the mode of administration, the requirements of an individual, the severity of the disease or condition being treated, and the judgment of the practitioner.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index which is expressed as the ratio between LD₅₀ and ED₉₀. PIM inhibitors exhibiting high therapeutic indices are preferred. The data obtained from cell culture assays and animal studies are optionally used in formulating a range of dosage for use in human. The dosage of such PIM inhibitors lies preferably within a range of circulating concentrations that include the ED₅₀ with minimal toxicity. The dosage optionally varies within this range depending upon the dosage form employed and the route of administration utilized.

### Assays for Identification and Characterization of PIM3 Inhibitors

Small molecule PIM inhibitors are optionally identified in high-throughput in vitro or cellular assays as described, for example, in U.S. Pat. Nos. 8,283,356 B2, 7,671,063 B2, and 8,431,589 B2. PIM inhibitors suitable for the methods described herein are available from a variety of sources including both natural (e.g., bacterial culture, soil or plant extracts) and synthetic. For example, candidate PIM inhibitors are isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks, as desired. Theoretically, the systematic, combinatorial mixing of 100 interchangeable chemical building blocks results in the synthesis of 100 million tetrameric compounds or 10 billion pentameric compounds (See, for example, Gallop, M. A., et al., J. Med. Chem, 1994, 37(9), 1233-1251). Each member of a library may be singular and/or may be part of a mixture (e.g a “compressed library”). The library may comprise purified compounds and/or may be “dirty” (i.e., containing a quantity of impurities). Preparation and screening of combinatorial chemical libraries are documented methodologies (See: Cabilly, S. ed., Combinatorial Peptide Library Protocols in Methods in Molecular Biology, Humana Press, Totowa, N.J., (1998)). Combinatorial chemical libraries include, but are not limited to: diversomers such as hydantoins, benzodiazepines, and dipeptides, as described in, e.g., DeWitt, S H, et al., Proc. Natl. Acad. Sci. USA., 1993, 90, 6909-4913; analogous organic syntheses of small compound libraries, as described in Chen, C., et al., J. Am. Chem. Soc., 1994, 116, 2661-2662; Oligocabamates, as described in Cho, C. Y., et al., Science, 1993, 261, 1303-1305: peptidyl phosphonates, as described in Campbell, D. A., Bernak, J. C., J. Org. Chem., 1994, 59, 658-660; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Devices for the preparation of combinatorial libraries we commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of robotic systems have also been developed for solution phase chemistries. These system include automated workstations and automated synthesis systems, such as the Microlab NIMBUS, Microlab VANTAGE, and Microstar systems developed by Hamilton, Inc. (Reno, Nev.), and the FLEX ISYNTH system developed by Chemspeed Technologies, Inc. (New Brunswick, N.J.), as well as many robotic systems utilizing robotic arms (e.g., Staubli). Any of the above devices are optionally used to generate combinatorial libraries for identification and characterization of PIM inhibitors which mimic the manual synthetic operations performed by small molecule PIM inhibitors suitable for the methods described herein. Any of the above devices are optionally used to identify and characterize small molecule PIM inhibitors suitable for the methods disclosed herein.

The identification of potential PIM inhibitors is determined by, for example, assaying the in vitro kinase activity of PIM kinases in the presence of candidate inhibitors. In such assays, PIM and/or a characteristic PIM fragment produced by recombinant means is contacted with a substrate in the presence of a phosphate donor (e.g., ATP)containing radiolabeled phosphate, and PIM-dependent incorporation is measured. “Substrate” includes any substance containing a suitable hydroxyl moiety that can accept the y-phosphate group from a donor molecule such as ATP in a reaction catalyzed by PIM. The substrate may be an endogenous substrate of PIM, i.e. a naturally occurring substance that is phosphorylated in unmodified cells by naturally-occurring PIM3 (e.g., BAD or Cdc25A)) or any other substance that is not normally phosphorylated by PIM in physiological conditions, but may be phosphorylated in the employed conditions. The substrate may be a protein or a peptide, and the phosphorylation reaction may occur on a serine and/or threonine residue of the substrate. For example, specific substrates, which are commonly employed in such assays include, but are not limited to, histone proteins and myelin basic protein. In some embodiments, PIM3 inhibitors are identified using IMAP® technology or LanthaSoreen technology.

Detection of PIM dependent phosphorylation of a substrate can be quantified by a number of means other than measurement of radiolabeled phosphate incorporation. For example, incorporation of phosphate groups may affect physiochemical properties of the substrate such as electrophoretic mobility, chromatographic properties, light absorbance, fluorescence, and phosphorescence. Alternatively, monoclonal or polyclonal antibodies can be generated which selectively recognize phosphorylated forms of the substrate from non-phosphorylated forms whereby allowing antibodies to function as an indicator of PIM3 kinase activity.

High-throughput PIM kinase assays can be performed in, for example, microtiter plates with each well containing PIM kinase or an active fragment thereof, substrate covalently linked to each well, P³² radiolabeled ATP and a potential PIM inhibitor candidate. Microtiter plates can contain 96 wells or 1536 wells for large scale screening of combinatorial library compounds. After the phosphorylation reaction has completed, the plates are washed leaving the bound substrate. The plates are then detected for phosphate group incorporation via autoradiography or antibody detection. Candidate PIM inhibitors are identified by their ability to decrease the amount of PIM3 phosphotransferase ability upon a substrate in comparison with PIM phosphotransferase ability alone.

The identification of potential PIM inhibitors may also be determined, for example, via in vitro competitive binding assays on the catalytic sites of PIM such as the ATP binding site and/or the substrate binding site. For binding assays on the ATP binding site, a known protein kinase inhibitor with high affinity to the ATP binding site is used such as staurosporine. Staurosporine is immobilized and may be fluorescently labeled, radiolabeled or in any manner that allows detection. The labeled staurosporine is introduced to recombinantly expressed PIM protein or a fragment thereof along with potential PIM3 inhibitor candidates. The candidate is tested for its ability to compete, in a concentration-dependent manner, with the immobilized staurosporine for binding to the PIM protein. The amount of staurosporine bound PIM is inversely proportional to the affinity of the candidate inhibitor for PIM kinases. Potential inhibitors would decrease the quantifiable binding of staurosporine to PIM (See, e.g., Fabian, M. A., et al., Nat Biotech., 2005, 23, 329-336). Candidates identified from this competitive binding assay for the ATP binding site for PIM3 would then be further screened for selectivity against other kinases for PIM kinase specificity.

The identification of potential PIM inhibitors may also be determined, for example, by in cyto assays of PIM activity in the presence of the inhibitor candidate. Various cell lines and tissues may be used, including cells specifically engineered for this purpose. In cyto screening of inhibitor candidates may assay PIM activity by monitoring the downstream effects of PIM activity as well as other cellular responses such as growth, growth arrest, differentiation, or apoptosis.

Alternatively, PIM-mediated phosphorylation of a downstream target of PIM can be observed in cell based assays by first treating various cell lines or tissues with PIM inhibitor candidates followed by lysis of the cells and detection of PIM mediated events. Cell lines used in this experiment (e.g., liver cancer cell lines such as HepG2, HepaRG, Huh7, and Hep3b) may include cells specifically engineered for this purpose. PIM mediated events include, but are not limited to, PIM mediated phosphorylation of downstream PIM mediators. For example, phosphorylation of downstream PIM mediators can be detected using antibodies that specifically recognize the phosphorylated PIM mediator but not the unphosphorylated form. These antibodies have been described in the literature and have been extensively used in kinase screening campaigns.

Numerous contract research organizations (CROs) offer PIM kinase assay services, including DiscoverX, Inc, (San Diego, Calif.), Reaction Biology Corporation (Malvern, Pa.), ChemDiv (San Diego, Calif.), and Cams Biosciences (Tokyo, Japan).

The identification of potential PIM inhibitors may also be determined, for example, by in vivo assays involving the use of animal models, including transgenic animals that have been engineered to have specific defects or carry markers that can be used to measure the ability of a candidate substance to reach and/or affect different cells within the organism. For example, mice have been engineered to overexpress PIM, leading to a disease, such as a malignant tumor, that can be treated with a PIM inhibitor.

In accordance with the foregoing, the present disclosure also provides:
(1) A compound of Formulas (I)-(IX), or a pharmaceutically acceptable salt thereof, for use as a pharmaceutical;
(2) A compound of Formulas (I)-(IX), or a pharmaceutically acceptable salt thereof, for use as a PIM inhibitor, for example for use in any of the particular indications hereinbefore set forth;
(3) A pharmaceutical composition, e.g. for use in any of the indications herein before set forth, comprising a compound of Formulas (I)-(IX), or a pharmaceutically acceptable salt thereof, together with one or more pharmaceutically acceptable diluents or carriers therefor.
(4) A method for the treatment of any of particular indication hereinbefore set forth in a subject in need thereof which comprises administering to the subject an effective amount of a compound of Formulas (I)-(IX), or a pharmaceutically acceptable salt thereof;
(5) The use of a compound of Formulas (I)-(IX) or e pharmaceutically acceptable salt thereof, for the manufacture of a medicament for the treatment or prevention of a disease or condition in which PIM3 activation plays a role or is implicated; e.g. as discussed above. The compounds of Formula (I)-(V) may be administered as the sole active ingredient or in conjunction with, e.g. as an adjuvant to, other drugs e.g, in immunosuppressive or immunomodulating regimens or other anti-inflammatory agents, e.g. for the treatnent or prevention of allo- or xenograft acute or chronic rejection or inflammatory or autoimmune disorders, a chemotherapeutic agent or an anti-infective agent, e.g. an anti-viral agent such as e.g. an anti-retroviral agent or an antibiotic. For example, the compounds of Formula (I) may be used in combination with a calcineurin inhibitor, e.g. cyclosporin A, ISA 247 or FK 506; an mTOR inhibitor, e.g. rapamycin, CC1779, ABT578, biolimus-7, biolimus-9, TAFA-93, AP23573, AP23464, or AP23841; an ascomycin having immunosuppressive properties, e.g. ABT-281, ASM981, etc.; corticosteroids; cathepsin S inhibitors; cyclophosphamide; azathioprine; methotrexate; leflunomide; mizoribine; mycophenolic acid; mycophenolate mofetil; 5-deoxyspergualine or an immunosuppressive homologuc, analogue or derivative thereof; a sphingosine-1-phosphate receptor agonist, e.g. FTY720 or an analog thereof e.g Y-36018; monoclonal antibodies to leukocyte receptors, e.g., MHC, CD2, CD3, CD4, CD7, CD8, CD11a/CD18, CD25, CD27, CD28, CD40, CD45, CD58, CD80, CD86, CD137, ICOS, CD150 (SLAM), OX40, 4-1BB or to their ligands, e.g. CD154, or antagonists thereof; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists, e.g. natalizumab (ANTEGREN®); or antichemokine antibodies or antichemokine receptor antibodies or low molecular weight chemokine receptor antagonists, e.g. anti MCP-1 antibodies.

A compound of Formulas (I)-(IX) may also be used in combination with other antiproliferative agents. Such antiproliferative agents include, but are not limited to:
(i) aromatase inhibitors, e.g. steroids, especially exemestane and formestane ad, in particular, non-steroids, especially aminoglutethimide, vorozole, fadrozole, anastrozole and, very especially, letrozole;
(ii) antiestrogens, e.g. tamoxifen, fulvestrant, raloxifene and raloxifene hydrochloride;
(iii) topoisomerase II inhibitors, e.g. topotecan, irinotecan, 9-nitrocamptothecin and the macromolecular camptothecin conjugate PNU-166148 (compound A1 in W099/17804);
(iv) topoisomerase II inhibitors, e.g. the anthracyclines doxorubicin (including liposomal formulation, e.g. CAELYX™), epirubicin, idarubicin and nemorubicin, the anthraquinones mitoxantrone and losoxantrone, and the podophyllotoxins etoposide and teniposide;
(v) microtubule active agents, e.g. the taxanes paclitaxel and docetaxel, the vinca alkaloids, e.g., vinblastine, especially vinblastine sulfate, vincristine especially vincristine sulfate, and vinorelbine, discodermolide and epothilones, such as epothilone B and D;
(vi) alkylating agent, e.g. cyclophosphamide, ifosfamide and melphalan;
(vii) histone deacetylase inhibitors;
(viii) farnesyl transferase inhibitors;
(ix) COX-2 inhibitors, e.g. celecoxib (CELEBREX®), rofecoxib (VIOXX®) and lumiracoxib (COX189);
(x) MM inhibitors;
(xi) mTOR inhibitors;
(xii) antineoplastic anti metabolites, e.g. 5-fluorouracil, tegafur, capecitabine, cladribine, cytarabine, fludarabine phosphate, fluorouridine, gemcitabine, 6-mercaptopurine, hydroxyurea, methotrexate, edatrexate and salts of such compounds, and furthermore ZD 1694 (RALTITREXED™, LY231514 (ALIMTA™), LY264618 (LOMOTREKOL™) and OGT719;
(xiii) platin compounds, eg carboplatin, cis-platin and oxaliplatin;
(xiv) compounds decreasing the protein kinase activity and further anti-angiogenic compounds, e.g. (i) compounds which decrease the activity of the Vascular Endothelial Growth Factor (VEGF)(b) the Epidermal Growth Factor (EGF), c-Src, protein kinase C, Platelet-derived Growth Factor (PDGF), Br-Ab tyrosine kinase, e-kit, Flt-3 and Insulin-like Growth Factor 1 Receptor (GF-IR) and Cyclin-dependent kinases (CDKs);
(ii) Imatinib, midostaurin, IRESSA™ (ZD1839), COP 75166, vatalanib, ZD6474, GW2016, CHIR-200131, CEP-7055/CEP-5214, CP-547632 and KRN-633; (iii) thalidomide (THALOMID), celecoxib (Celebrex), SU5416 and ZD6126;
(xv) gonadorelin agonists, e.g. abarelix, goserelin and goserelin acetate;
(xvi) anti-androgens, e.g. bicalutamide (CASODEX™);
(xvii) bengamides;
(xviii) bisphosphonates, e.g. etridonic acid, clodronic acid, tiludronic acid, pamidronic acid, alendronic acid, ibandronic acid, risedronic acid and zoledronic acid;
(xix) antiproliferative antibodies, e.g. trastuzumab (HERCEPTIN), Trastuzumab-DM1, erlotinib (TARCEVA™), bevacizumab (AVASTIN™, rituximab (RITUXAN®), PRO64553 (anti-CD40) and 2C4 Antibody;
(xx) temorolomide (TEMODAL®).

The structure of the active agents identified by code nos., generic or trade names may be taken from the actual edition of the standard compendium “The Merck Index” or from databases, e.g. Patents International (e.g, IMS World Publications).

In accordance with the foregoing the present disclosure provides in a yet further aspect:

(6) A method as defined above comprising co-administration, e.g., concomitantly or in sequence, of a therapeutically effective amount of (a) a compound of Formulas (I)-(IX), or acceptable salt thereof, and b) a second drug substance, said second drug substance being, for example, for use in any of the particular indications hereinbefore set forth.
(7) A combination comprising a therapeutically effective amount of a PIM kinase inhibitor, e.g. a compound of Formula (I) and/or (II) or a pharmaceutically acceptable salt thereof, and a second drug substance, said second drug substance being for example as disclosed above. Where a PIM kinase inhibitor, e.g. a compound of Formula (I) and/or (II), is administered in conjunction with other immunosuppressive, immunomodulatory, anti-inflammatory or antineoplastic agent, e.g. as disclosed above, dosages of the on-administered drug or agent will of course very depending on the type of co-drug or agent employed, or the specific drug or agent used, or the condition being treated and so forth.

### Cell-Free Biosynthesis

In some embodiments, methods and systems for synthesis of compounds and compositions of the present disclosure, including PIM inhibitors, are in vitro cell-free biosynthesis (CFB) systems that serve as a platform to produce proteins and small molecule metabolites using the cells enzymes and metabolic machinery without the living cell (See: Hodgman, C. B., Jewett, M. C., Metab. Eng., 2012, 14(3), 261-269). Cell-free biosynthesis systems provided herein have numerous applications for drug discovery by allowing rapid expression of natural biosynthetic genes and pathways and by allowing activity screening without the need for plasmid based cloning and in vivo propagation, thus enabling rapid process/product pipelines (creation of small molecule libraries). A key feature of the CFB methods and systems used herein is that biosynthesis pathway flux to a target compound can be optimized by directing resources to user defined objectives and consequently allows for the exploration of a large sequence space. Central metabolism, oxidative phosphorylation, and protein synthesis can be co-activated by the user. The lack of a cell wall also provides for the ability to easily screen toxic metabolites, proteins, and small molecule. Cell-free biosynthesis methods involving in vitro transcription/translation (TX-TL) have been used to produce (1) proteins (See, for example: Carlson, E. D., et al., Biotechnol Adv., 2012, 30(5), 1185-1194; Swartz, J., et al., U.S. Pat. No. 7,338,789; Goerke, A. R., et al, U.S. Pat. No. 8,715,958), (2) antibodies and antibody analogs (See, for example: Zimmerman, E. S., et at, Bioconjugate Chem., 2014, 25, 351-361; Thanos, C. D., et al., US Patent No. 2015/0017187 A1), and (3) small molecules (See, for example: Kay, J., et al., Metabolic Engineering, 2013, 32, 133-142; Goering, A. W, ea at, ACS Synth Bio., 2017, 6(1), 39-44; Blake, W J., et al., U.S. Pat. No. 9,469,861).

The CFB methods and systems can be used to rapidly prototype novel complex biocircuits as well as metabolic pathways. Protein expression from multiple DNA pieces, including linear and plasmid based DNA, can be performed. The CFB methods and systems enable modulating concentrations of DNA encoding individual pathway enzymes and testing the related effect on metabolite production. The ability to express multi-enzyme pathways using linear DNA in the CFB methods and systems bypasses the need for in vivo selection and propagation of plasmids. Linear DNA fragments can be assembled in 1 to 3 hours (hrs) via isothermal or Golden Gate assembly techniques and be immediately used for a CFB reaction. The CFB reaction can take place in several hours, e.g. approximately 4-8 hours, or may be run for longer periods up to 48 hours. The use of linear DNA provides a valuable platform for rapid prototyping libraries of DNA/genes. In the CFB methods and systems, mechanisms of regulation and transcription exogenous to E. coli, such as the tet repressor and T7 RNA polymerase, or other host cell extracts, can be supplemented as defined by the user to generate and maximize endogenous properties, diversity or production. The CFB methods and systems further enhance diversity and production of target compounds by modifying endogenous properties including mRNA and DNA degradation rates. ATP regeneration systems that allow for the recycling of inorganic phosphate, a strong inhibitor of protein synthesis, are manipulated in the CFB methods and systems Redox potential, including e.g., NAD/NADH, NADP/NADPH, are regenerated in CFB, and methods for modifying redox and availability of specific cofactor which in turn enables the user to selectively modulate any reaction in the CFB system.

In alternative embodiments, CFB methods and systems enable in vitro cell-free transcription/translation systems (TX-TL) and function as rapid prototyping platforms for the synthesis, modification and identification of products, e.g., natural products (NPs) or natural product analogs (NPAs), from biosynthetic pathway genes. In alternative embodiments, CFB systems are used for the combinatorial biosynthesis of natural products and natural product analogs, such as those provided in the present disclosure.

In alternative embodiments, CFB systems are used for the rapid prototyping of complex biosynthetic pathways as a way to rapidly assess combinatorial designs for the synthesis of compounds of Formulas (I)-(IX). In alternative embodiments, these CFB systems are multiplexed for high-throughput automation for rapid prototyping of natural product pathway genes, the natural products they encode and synthesize, and natural product analogs, such as the compounds of Formulas (I)-(IX) provided in the present disclosure. The CFB methods and systems are described in Culler, S. et al. PCT Application WO20171031399 A1, and is incorporated herein by reference.

As described herein, the CFB compositions, methods, and systems can be used to rapidly produce analogs of known compounds, for example natural product analogs and secondary metabolic structural analogs, such as compounds of Formulas (I)-(IX). Accordingly the CFB methods can be used in the processes described herein that generate product diversity. In some embodiments, methods provided herein include a cell-free (in vitro) biosynthesis (CFB) method for making, synthesizing or altering the structure of compounds of Formulas (I)-(IX). The CFB methods can produce in the TX-TL extract or extract mixture at least two or more of the altered compounds to create a library of altered compounds; preferably the library is a natural product analog library, prepared, synthesized or modified by the CFB method.

In alternative embodiments, practicing this disclosure comprises use ofany conventional technique commonly used in molecular biology, microbiology, and recombinant DNA, which are within the skill of the art. Such techniques are known to those of skill in the art and are described in numerous texts and reference works (See e.g., Sambrook et al., “Molecular Cloning: A Laboratory Manual,” Second Edition, Cold Spring Harbor, 1989; and Ausubel et al., “Current Protocols in Molecular Biology,” 1987). Unless defined otherwise herein, all technical and scientific terms used herein have the same meaning as commonly understood by one of ordinary skill in the art to which this disclosure pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, N.Y. (1991) provides those of skill in the art with general dictionaries of many of the terms used in this disclosure. Although any methods and materials similar or equivalent to those described herein find use in the practice of the present disclosure, the preferred methods and materials are described herein. Accordingly, the terms defined immediately below am more fully described by reference to the Specification as a whole.

Unless defined otherwise herein, all technical and scientific terms used herein have the same meaning as commonly understood by one of ordinary skill in the art to which this disclosure pertains. Although any methods and materials similar or equivalent to those described herein find use in the practice of the present disclosure, the preferred methods and materials are described herein. Accordingly, the terms defined immediately below are more fully described by reference to the Specification as a whole.

As used herein, the singular terms “a,” “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this disclosure is not limited to the particular methodology, protocols, and reagents described, as these may vary, depending upon the context they are used by those of skill in the art.

### Methods of Synthesis

Numerous methods are available for the synthesis of compounds such as those represented by Formulas (I)-(IX). Some of these methods are reviewed in Rao, B. P. C., et al, Strategies Towards the Synthesis of Staurosporine Indolocarbazole Alkaloid and Its Analogues in Scope of Selective Heterocycles from Organic and Pharmaceutical Perspective. Chapter 4, Intech Publishers; Rijeka, Croatia (EU), 2016. See also: Wilson, L J. et al., US Patent Application No. US 200710249590 A1; Kleinschroth, J. et al., U.S. Pat. No. 5,438,050; Kleinschroth, J. et al., U.S. Pat. No. 5,489,608; Faul, M M., et al., U.S. Pat. No. 5,665,877; Faul, M. M., et al., U.S. Pat. No. 5,919,946; Faul, M M, et al., U.S. Pat. No. 5,614,647; Faul, M M, et al., U.S. Pat. No. 6,037,475.

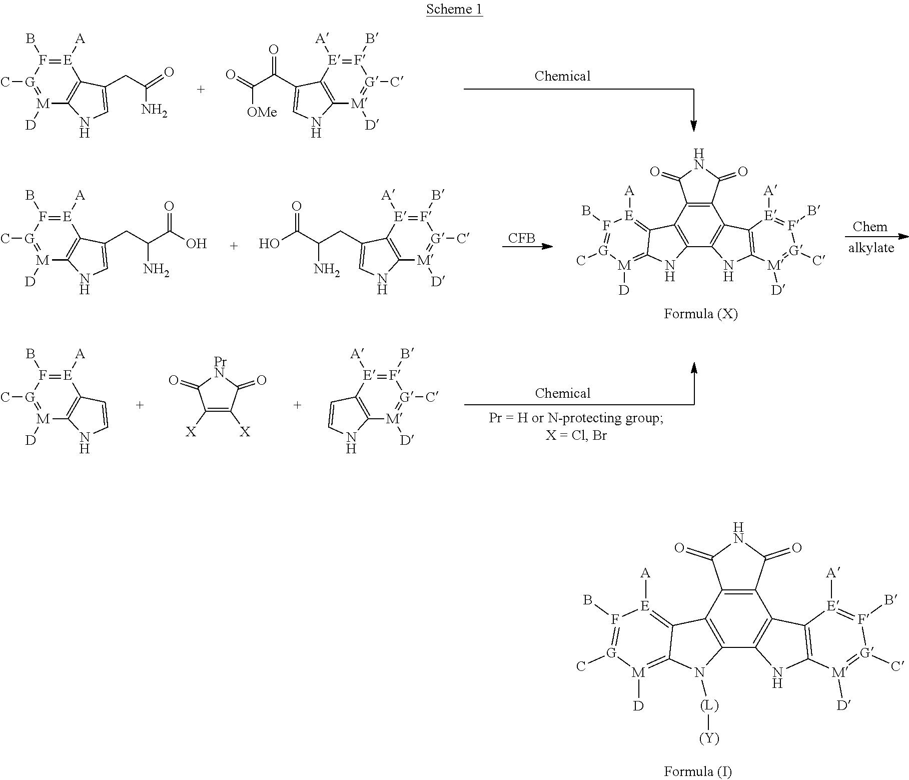

In one embodiment, compounds of Formula (I) may be prepared by reacting indole-3-acetamide derivatives with methyl indole-3-glyoxylates in the presence of potassium tert-butoxide in THF solvent, as shown in Scheme 2 and as reported in the literature (See, for example: Faul, ex al., Tetrahedron Lett., 1999, 40, 1109-1112; Paul et al., J Org. Chem. 1998, 63, 6053-6058; Faul et al., J. Org. Chem. 1999, 64, 2465-2470). Indole-3-acetamide and indole-3-glyoxylate derivatives are readily prepared from a wide range of available substituted indoles. Ring closure of the initially formed bisindolomaleimide derivatives illustrated by Formula (XI) affords the indolo[2,3-a]carbazoles represented by Formula (X), by treatment with a variety oxidants [O], including Pd(OAc)₂, PdCl₂, hv/O₂ or I₂, DDQ, CuCl₂, or Pd(OTf)₂ (See, for example: Paul et al., J. Org. Chem. 1999, 64, 2465-2470). Subsequent alkylation of Formula (X) with reactants such as 2-chloroethylamines then yields the desired Formulas (I).

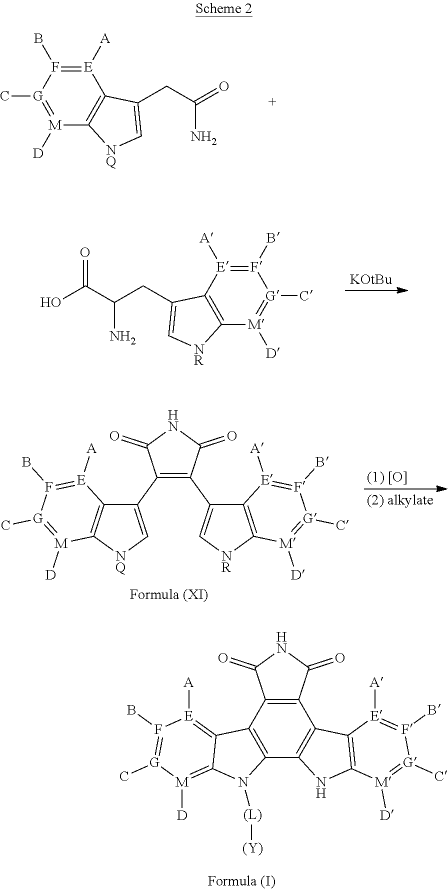

In another embodiment, compounds of Formula (XI) may be prepared by sequentially reacting substituted indoles, which are metallated with Mg or other metals, with 3,4-dihalosuccinimide or its N-protected form, as shown in Scheme 3 (X=Cl, Br) and as described in the literature (See, for example: Faul et al., Synthesis, 1995, 1511-1516; Gallant at al., J Org. Chem. 1993, 59, 343-349). Compounds of Formula (I) are then prepared by oxidation, as described above for Scheme 2. In both Scheme 2 and Scheme 3, the imide functionality can be reduced to the lactam functionality using standard reagents such as sodium borohydride or zinc amalgam. In Scheme 3, Q and/or R groups can be added by standard methods through reactions with the indole N—H functionality (e.g., through alkylation or acylation reactions).

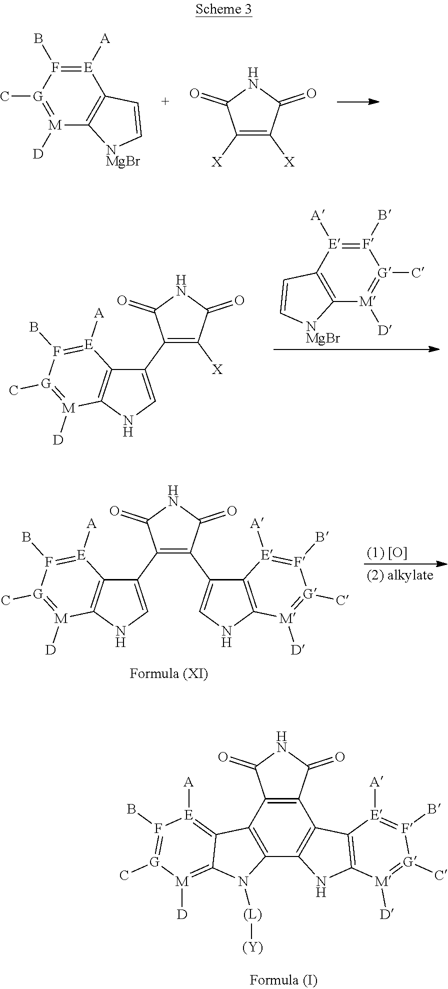

In another embodiment, compounds of Formulas (X) and (XI) may be prepared by reacting tryptophan derivatives in a process involving cell-free biosynthesis (CFB) as shown in Scheme 4. In this biological process, enzymes are used to condense two tryptophan molecules or tryptophan derivatives to directly produce compounds of Formula (I) where Q and R are hydrogen. The enzymes required for these transformations have been elucidated and enzymes from various pathways can be used to generate Indolocarbazole derivatives. Certain enzymes are known to catalyze transformations and facilitate pathways to produce natural indolocarbazole. These enzymes include, by way of example for Formula (X) using process CFB-1: VioA (amino oxidase) and VioB (chromopyrrolic acid synthase) of the violacein pathway, StaO (amino oxidase), StaD (chromopyrrolic acid synthase), StaP (cytochrome P450 monooxygenase), and StaC (flavin hydroxylase) of the staurosporine pathway, and RebO (amino oxidase), RebD (chromopyrrolic acid synthase), RebP (cytochrome P450 monooxygenase), and RebC (flavin hydroxylase) of the rebeccamycin pathway, or homologues thereof (See, for example: Sanchez t al., Nat. Prod. Rep. 2006, 23, 1007-1045; Sanchez et at, Proc. Natl. Acad. Sci. U.S.A., 2005, 102, 461-466; Du et al., ACS Synth. Biol. 2015, 4, 682-688; Du et al., Curr. Opin. Chem. Bio., 2016, 31, 7441). Similarly, compounds of Formula (XI) may be produced using process CFB-2, for example, using the enzymes Vio A and VioB, StaO and StaD, RebO and RebD, or homologues thereof, in combination with the enzyme MarC of the methylarcyriarubin pathway or homologues thereof (See: Chang, F.-Y. and Brady, S. F., ChemBioChem. 2014, 15(6), 815-821). These enzymes have been used herein to directly produce compounds of Formulas (X) and (XI), wherein A, B, C, D, A′, B′, C′, D, Q and R are hydrogen, and B, F, G, M, E′, F′, G′, M′ are carbon when tryptophan is used as a precursor. These enzymes can be used in engineered living cells, or alternatively in a cell-free process, to produce compounds of Formula (I) by chemical alkylation of the cell-free reaction products of Formula (X), Cell-free biosynthesis of natural product-like compounds is described in Culler, S. et al., PCT Application WO207/031399 A1, and is incorporated herein by reference.

In one embodiment of the present disclosure, the enzymes VioA and Vio B of the violacein pathway, StaO, StaD, StaP, and StaC of the staurosporine pathway, and/or RebO, RebD, RebP, and RebC of the rebeccamycin pathway, and/or MarC of the methylarcyriarubin pathway are used in a cell-free biosynthesis process to produce compounds of Formulas (X) or (XI) by combining and transforming two of the same or different substituted tryptophan derivatives, as outlined in Scheme 4. Rebeccamycin pathway enzymes (RebO, RebD, RebP, and RebC) can be used to directly produce compounds of Formula (X) or Formula (X).

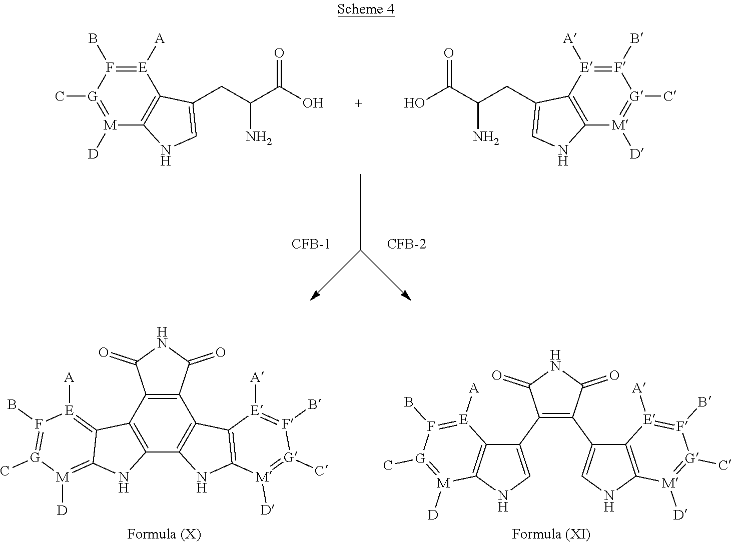

In another embodiment of the present disclosure, compounds of Formulas (X) or (XI), may be transformed through a chemical process to introduce a heteroatom-containing tail attached to the indole N atoms, as represented by Formulas (I).

### Examples

### General Methods

Examples related to the present disclosure are described below. In most cases, alternative techniques can be used. The examples ae intended to be illustrative and ae not limiting or restrictive to the scope of this disclosure. For example, where additional compounds are prepared following a protocol of a Scheme for a particular compound, it is understood that conditions may vary, for example, any of the solvents, reaction times, reagents, temperatures, work up conditions, or other reaction parameters may be varied. All molecular biology and cell-free biosynthesis reactions were conducted using standard plates, vial, and flasks typically employed when working with biological molecules such as DNA, RNA and proteins. All synthetic chemistry was performed in standard laboratory glassware and equipment unless indicated otherwise in the examples.

Commercial reagents were used as received. LC/MS data was collected using either a Shimadzu SCL10Avp HPLC system connected to a mass separation detector (PE SCIEX API 150EX) and equipped with an autosampler (Gilson 215), or an Applied Biosystems 3200 APCI triple quadrupole mass spectrometer with alternating positive and negative ion scans. High resolution mass spectrometry was performed using a Thermo Fisher Q Exactive MS instrument. Exactive HR MS: ESI in negative and/or positive ionization mode, XIC±10 ppm around exact mass (m/z); GC-MS was performed using an Agilent 6890N instrument equipped with a 5973N inert mass selective detector. All microwave irradiation experiments were carried out in a microwave reactor (Biotage Initiator EXP EU 355301) operating at the 2.45 GHz frequency with a continuous irradiation at the 300W maximal powder. Photochemical reactions were conducted using a high-pressure mercury lamp (LISMA, DRL-400 E40). Ion chromatography was performed using a Metrohm 940 Professional IC Vario instrument. Microwave reactions were performed in a Biotage Initiator using the instrument software to control heating time and pressure. Hydrogenation reactions were performed on an H-Cube using the commercially available catalyst cartridges. Silica gel chromatography was performed either manually using standard columns or using pre-packed Sep-Pak silica cartridges from Waters, Thin layer chromatography (TLC) analyses were performed using aluminum foil backed silica gel plates 60 F₂₅₄ silica (Sorbfil, Russia). Column chromatography (CC) was carried out using Merck 60 (70-230 mesh) silica. Preparative HPLC was performed on a Waters 1525/2487 with UV detection at 220 nm and manual collection. ¹H NMR was performed on a Jeol JNM-ECS-400 at 400 MHz or a Bruker DRX-600 at 600 MH, or a Bruker DPX-400 at 400 MHz and was referenced to a solvent residual peak, either CDCl₃ at 7.26 ppm or DMSO-d6 at 2.54 ppm, for ¹H-NMR, respectively. 6-Azaindole, 6-benzyloxyindole and 3-fluoro-4-hydroxybenzaldehyde were purchased from Combi-Blocks (San Diego, Calif., USA). Abbreviations are as follows: RT or t is room temperature; THF is tetrahydrofuran; EtOAc is ethyl acetate; IA is trifluoroacetic acid; Et₂O is diethylether; DCM is dichloromethane; ppm is parts per million; s=singlet; d=doublet; t=triplet; m=multiplet; dd=doublet of doublets; br=broad;

### Example 1. Synthesis of 3,4-dibromo-1-(2,4-dimethoxybenzyl)-1H-pyrrole-2,5-dione (3)

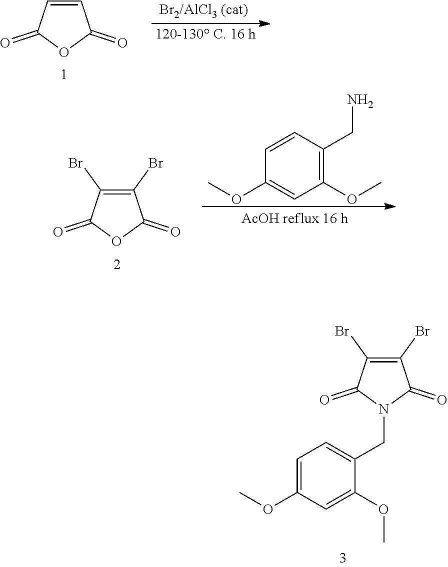

Step 1: Synthesis of 3,4-dibromo-2,5-furandione, intermediate (2). A flask under an argon atmosphere was charged with maleic anhydride (3.00 g, 30.6 mmol), bromine (3.15 ml, 61.2 mmol), and aluminum chloride (0.200 g, 1.3 mmol). The flask was sealed, the reaction mixture was heated to 120-130° C., and maintained at this temperature for 16 h After cooling down to rt the mixture was dissolved in EtOAc (50 ml) and filtered. The filtrate was evaporated in vacuo to dryness to afford crude compound 2 (10.1 g) as a mixture of a light-orange oil and colorless crystals. The crude product was used for the next step without further purifications.

Step 2: Synthesis of 3,4-dibromo-1-(2,4-dimethoxybenzyl)-1H-pyrrole-2,5-dione (3). To a stirred solution of crude compound 2 (10.1 g) in acetic acid (50 ml) a 2,4-dimethoxybenzylamine was added dropwise at rt under Ar. The mixture was refluxed for a 16 h under Ar. The solution was evaporated to dryness, a residue was dissolved in EOAc (100 ml), washed with 10% aq sodium bicarbonate, water, and brine. The solution was dried over sodium sulfate, evaporated in vacuo to dryness, and purified via column chromatography ((silica gel, eluent DCM/CCl₄ 1:1 to DCM 100%) to afford compound 3 (3.8 g, 31% combined yield over 2 steps) as a yellow solid. Product purity was confirmed by ¹H-NMR (400 MHz, CDCl₃): δ (ppm) 7.21 (d, 1H), 6.41-6.45 (m, 2H), 4.74 (s, 2H), 3.85 (s, 3H), 3.76 (s, 3).

### Example 2. Synthesis of 6-(benzyloxy)-5-fluoro-1H-indole (8)

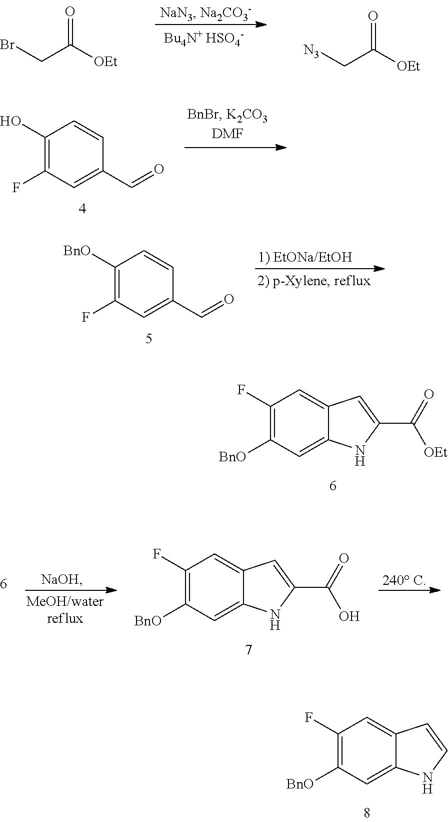

Step 1: Synthesis of intermediate ethyl azidoacetate. To a stirred solution of ethyl bromoacetate (55.0 mL, 0.5 mol) in toluene (200 mL) was added tetrabutylammonium hydrogen sulfate (3.3 g, 0.009 moo) and mixture was cooled to 5-10° C. Then a solution of sodium azide (34.0 g, 0.5 mol) and sodium carbonate (2.15 g, 0.02 mol) in water (100 mL) was added. The resulting mixture was stirred at ambient temperature for 3 h. Then the organic phase was separated, dried over sodium sulfate, and filtered. The resulting solution of ethyl azidoacetate was used for the next step without further purification.

Step 2: Synthesis of 3-fluoro-4-benzyloxybenzaldehyde (5). To a stirred solution of 3-fluoro-4-hydroxybenzaldehyde 4 (50 g, 0.35 mol) in DMF (0.5 L) were added K₂CO₃ (59.2 g, 0.43 mol) and benzyl bromide (67 g, 0.39 mol). The reaction mixture was stirred at 55° C. for 2 h (TLC control), then cooled, water (1.5 L) was added, and the formed precipitate was filtered, washed with small portions of DMF, water, and then dried to afford compound 5 (74 g, 90%). Final product purity was confirmed by ¹H-NMR (400 MHz, CDCl₃): δ (ppm) 9.96 (d, 1H), 7.56-7.68 (m, 2H), 7.31-7.53 (m, 5H), 7.13 (t, 1H), 5.25 (s, 2H).

Step 3: Synthesis of ethyl 6-(benzyloxy)-5-fluoro-1H-inode-2-carboxylate (6). Compound 5 (25.0 g, 0.1 mol) was dissolved in the solution of ethyl azidoacetate in toluene (see above), The mixture was slowly added to a cold (−20° C.) solution of sodium ethoxide (13.5 g, 0.44 mol) in ethanol (250 mL) within 80 min. The reaction mixture was stirred for 3 h at 0° C., filtered, and the precipitate was washed with small portions of ethanol. The solids then were partitioned between a saturated aqueous solution of ammonium chloride (1 L) and ethyl acetate (1 L). The aqueous layer was extracted with ethyl acetate and combined organic phases were dried over sodium sulfate, filtered, and concentrated in vacuo to dryness. The solid residue was suspended in p-xylene (200 mL) and refluxed for 2 h. The solvent was evaporated in vacuo to dryness. The residue was purified via column chromatography (silica gel, eluent DCM) to afford compound 6 (12.0 g, 44%). Final product purity was confirmed by ¹H-NMR (400 MHz, CDCl₃): δ (ppm) 8.90 (br, 1H), 7.44-7.51 (m, 2H), 732-7.43 (m, 4), 7.14 (d, 1H), 6.94 (d, 1H), 5.19 (s, 2H), 433-4.46 (m, 2H), 1.40 (t, 3H).

Step 4: Synthesis of 6-(benzyloxy)-5-fluoro-1H-indole-2-carboxylic acid (7). To solution of compound 6 (12 g, 0.04 mol) in methanol (100 mL) was added solution of NaOH (1.8 g, 0.08 mol) in water (50 mL). The solution was refluxed for 1 h (TLC control), evaporated under vacuum to dryness. The residue was resuspended in water (200 mL) and the solution was adjusted with HC to pH 3, the formed precipitate was filtered, washed with water, and dried to afford acid 7 (9.8 g, 90%). Final product purity was confirmed by ¹H-NMR (400 MHz, DMSO-d6): δ (ppm) 12.8 (br, 1H), 11.67 (s, 1H), 7.5 (d, 2H), 7.40 (m, 3), 7.35 (t, 1H), 7.09 (d, 1H), 7.01 (s, 1H), 5.18 (s, 2H).

Step 5: Synthesis of 6-(benzyloxy)-5-fluoro-1H-indole (8). The acid 7 (7.5 g, 0.0273 mol) was heated at 240° C. over an oil bath until completely melted, followed by heating for additional 10 min at this temperature. After cooling the residue was dissolved in a minimal volume of a mixture of hexane/DCM 2:3 and purified via column:chromatography (silica gel, eluent hexane/DCM 2:3) to afford 6-(benzyloxy)-5-fluoro-1H-indole 8 (3.6 g, 54%). Final product purity was confirmed by ¹H-NMR (400 MHz, CDCl₃): δ (ppm) 8.02 (br, 1H), 7.49 (d, 2H), 7.40 (t, 2H), 7.30-7.36 (m, 2H), 7.14 (t, 1H), 6.98 (d, 1H), 6.47 (t, 1H), 5.18 (s, 2H).

### Example 3. Synthesis of tert-butyl 3-(tributylstannyl)-1H-pyrrolo[2,3-c]pyridine-1-carboxylate (12)

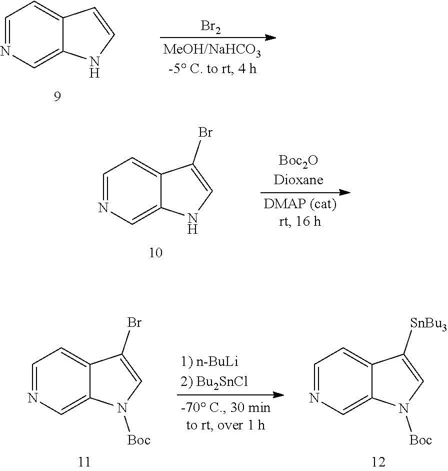

Step 1: Synthesis of 3-bromo-1H-pyrrolo[2,3-c]pyridine (10). To a stirred mixture of 6-azaindole 9 (2.30 g, 19.5 mmol) and sodium bicarbonate (4.91 g, 58.5 mmol) in MeOH (30 ml) a solution of bromine (3.12 g, 19.5 mmol) in MeOH (5 ml) was added dropwise at −5-0° C. The resulting mixture was stirred at rt for 4 h. The reaction mixture was evaporated to dryness in vacuo, the residue was dissolved in EtOAc (100 ml), the solution was washed with water and brine. The organic layer was dried over sodium sulfate and evaporated in vacuo to dryness to afford a crude product. The product was purified by recrystallization from mixture ether/hexane 1:1 to afford compound 10 (3.00 g, 78%) as a beige solid. Product purity was confirmed by ¹H-NMR (400 MHz, DMSO-d6): δ (ppm) 11.99 (br, 1H), 8.77 (s, 1H), 8.19 (dd, 1H), 7.82-7.81 (m, 1H), 7.40 (dd, 1H).

Step 2: Synthesis of tert-butyl 3-bromo-1H-pyrrolo[2,3-c]pyridine-1-carboxylate (11). To a stirred solution of compound 10 (3.00 g, 15.2 mmol) and catalytic amount of DMAP (150 mg) in dioxane (80 ml) was added at rt a solution of Boc-anhydride (4.00 g, 18.2 mmol) in dioxane (20 ml). The resulting solution was stirred at rt for 16 h, then evaporated in vacuo to dryness. The residue was purified via column chromatography (silica gel, eluent DCM/ether 8:1) to afford compound 11 (4.0 g, 88%) as a beige solid. Final product purity was confirmed by NMR and HPLC. ¹H-NMR (400 MHz, CDCl₃): δ (ppm) 9.41 (s, 1H), 8.51 (d, 1H), 7.81 (s, 1H), 7.50 (d, 1H), 1.71 (s, 9H). MS (ESI) m/z 299.3 [MH]+.

Step 3: Synthesis of tert-butyl 3-tributylstannyl)-1H-pyrol[2,3-c]pyridine-1-carboxylate (12). To a stirred solution of compound 11 (3.00 g, 10.1 mmol) and tributyltin chloride (19.70 g, 60.6 mmol) in THF (300 ml) a 2.5M solution of butyllithium in THF (20.0 ml, 50.5 mmol) was added dropwise at −70° C. under an argon atmosphere. The resulting solution was stirred at −70° C. for 30 min and then allowed to reach rt on its own accord. The reaction mixture was quenched with water (200 ml) and diluted with ether (200 ml) then the organic layer was separated, washed with water, brine, dried over sodium sulfate, and evaporated in vacuo to dryness. The residue was dissolved in ether (3 ml) and diluted with hexane (15 ml), then a formed precipitate was filtered (solid are the staring material 3). The filtrate was evaporated in vacuo to dryness, the residue was dissolved in CCl₄ and purified via column chromatography (silica gel, eluent EtOAc/hexane 1:10 to EtOAc/hexane 1:5) to afford compound 12 (2.00 g, 40%) as a pale-yellow oil. Final product purity was confirmed by ¹H-NMR (400 MHz, CDCl₃): δ (ppm) 9.38 (br, 1H), 8.39 (d, 1H), 7.68-7.73 (m, 1H), 7.48 (d, 1H), 1.72 (s, 9H), 1.52-1.60 (m, 6H), 1.31-1.40 (m, 6H), 1.15-1.19 (m, 6H), 0.9 (t, 9H).

### Example 4. Synthesis of Compounds 18 and 19 (Individual Isomers)

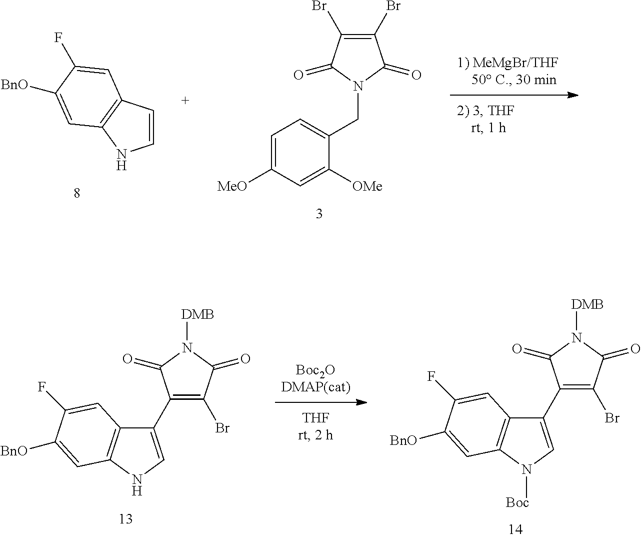

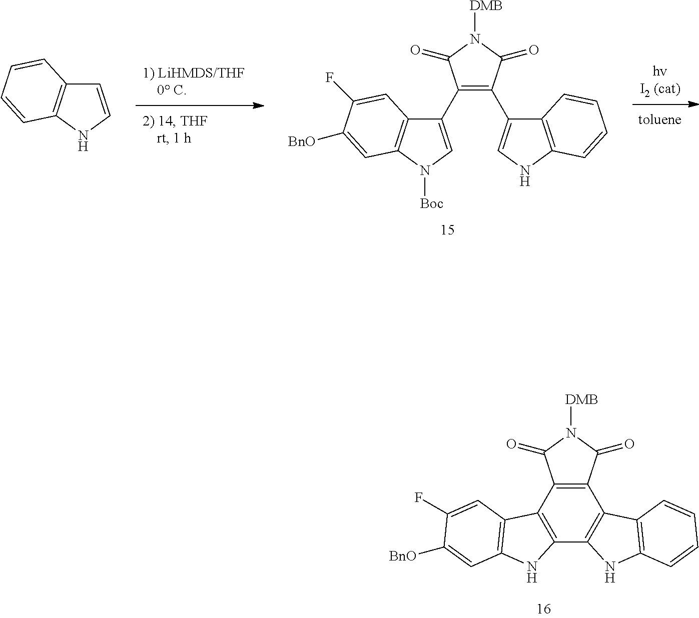

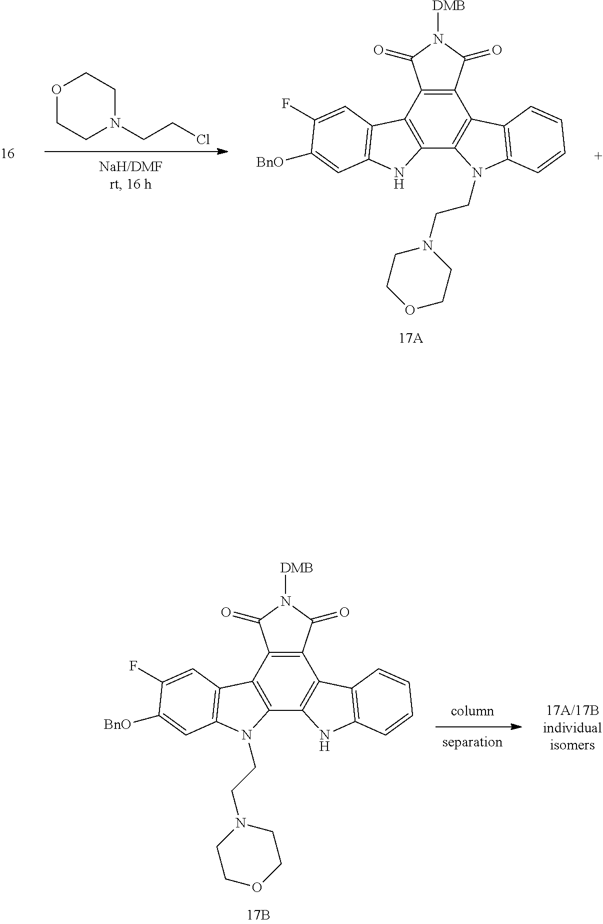

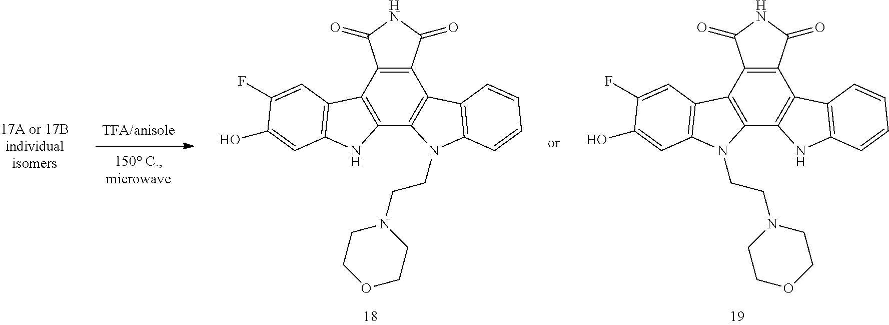 [SMILES: O=C1NC(=O)c2c1c1c3cc(F)c(O)cc3[nH]c1c1c2c2ccccc2n1CCN1CCOCC1 ; O=C1NC(=O)c2c1c1c3ccccc3[nH]c1c1c2c2cc(F)c(O)cc2n1CCN1CCOCC1]

Step 1: Synthesis of 3-[6-(benzyloxy)-5-fluoro-1H-indol-3-yl]-4-bromo-1-(2,4-dimethoxybenzyl)-1H-pyrrole-2,5-dione, compound 13. To a stirred solution of 4-(benzyloxy)-5-fluoro-1H-indole (8) (1.2 g, 4.9 mmol) in THF (20 ml) 1.4M methylmagnesium bromide solution in THF/toluene 1:3 (3.8 ml, 4.9 mmol) was added dropwise under an argon atmosphere at rt. The resulting dark solution was stirred at 40-50° C. for 1 h. The reaction mixture was cooled to rt and a solution of dibromomaleimide 2 (1.0 g, 2.5 mmol) in THF (10 ml) was added dropwise at it within 1 h under argon. The reaction mixture was stirred at ambient temperature for 1 h, and then was poured into an ice-cold 10% aq solution of citric acid (200 ml). The resulting mixture was extracted with EtOAc (2×50 ml, 4 an organic layer was washed with water, brine, dried over sodium sulfate and evaporated in vacuo to dryness. The residue was purified via column chromatography (silica gel, eluent 100% DCM to DCM/EtOAc 9:1) to afford compound 13 (12 g, 86%). Product purity was confirmed by ¹H-NMR (400 MHz, CDCl₃): δ (ppm) 8.71 (br, 1H), 7.96 (d, 1H), 7.86 (d, 1H), 7.43-7.50 (m, 2H), 7.30-7.43 (m, 4H), 7.22 (d, 1H), 6.97 (d, 1H), 6.37-6.54 (m, 1H), 5.18 (s, 2H), 4.80 (s, 2H), 3.84 (s, 3H), 3.79 (s, 3H).

Step 2: Synthesis of tert-butyl 6-(benzyloxy)-3-[4-bromo-1-(2,4-dimethoxybenzyl)-2,5-dioxo-2,5-dihydro-1H-pyrrol-3-yl]-5-fluoro-1H-indole-1-carboxylate, compound 14, To a stirred solution of compound 13 (1.2 g, 2.1 mmol) and DMAP (0.012 g, 0.1 mmol) in THF (20 ml) a solution of Boc₂O (0.5 g, 2.2 mmol) in THF (5 ml) was added at it under argon. The resulting solution was stirred at rt for 2 h, and then was evaporated in vacuo to dryness. The residue was purified via column chromatography (silica gel, eluent 100% DCM to DCM/EtOAc 9:1) to afford compound 14 (11 g, 85%). Product purity was confirmed by ¹H-NMR (400 MHz, CDCl₃): δ (ppm) 8.15 (s, 1H), 8.00 (d, 1H), 7.61 (d, 1H), 7.50 (d, 2H), 730-746 (m, 3H), 7.25 (d, 1H), 6.40-6.52 (m, 2H), 5.23 (s, 2H), 4.80 (s, 2H), 3.84 (s, 3H), 3.79 (S, 3), 1.68 (s, 9H).

Step 3: Synthesis of tert-butyl 6-(benzyloxy)-3-[1-(2,4-dimethoxybenzyl)-4-(1H-indol-3-yl)-2,5-dioxo-2,5-dihydro-1H-pyrrol-3-yl]-5-fluoro-1H-indole-1-carboxylate, compound 15. To a stirred solution of HMDS (0.5 g, 3.4 mmol) in THF (50 ml) a 2.5M solution of butyllithium in THF (1.4 ml, 3.4 mmol) was added dropwise at 0° C. under argon. The resulting solution was stirred for 30 min at 0° C. Then the solution was cooled down to −20° C. and a solution of 1H-indole (0.37 g, 3.1 mmol) in THF (5 ml) was added dropwise. The reaction mixture was stirred at −20° C. for 45 min. Then to the stirred solution was added dropwise a solution of the intermediate compound 14 (0.7 g, 1.0 mmol) in THF (20 ml) at −20° C. over 45 min under an argon atmosphere. The resulting mixture was stirred at −20° C. for 45 min, and additionally for 1 h at 0° C., then the resulting mixture was poured into an ice-cold 10% aq solution of citric acid (200 ml). The mixture was extracted with EtOAc (2×50 mL), the organic layer was washed with water, brine, dried over sodium sulfate, and evaporated in vacuo to dryness. The residue was purified via column chromatography (silica gel, eluent 100% DCM to DCM/EtOAc 9:1) to afford compound 15 (525 mg, 68%). Product purity was confirmed by ¹H-NMR (400 MHz, DMSO-d6): δ (ppm) 11.92 (br, 1H), 7.78-48.04 (m, 3H), 7.24-7.55 (m, 7H), 6.97-7.14 (m, 2H), 6.85 (d, 1H), 6.73 (t, 1H), 6.53-6.65 (m, 2H), 6.47 (d, 1H), 5.14 (s, 2H), 4.67 (s, 2H), 3.82 (s, 3H), 3.73 (s, 3H), 1.60 (s, 9H).

Step 4: Synthesis of tert-butyl 10-(benzyloxy)-6-(2,4-dimethoxybenzyl)-9-fluoro-5,7-dioxo-5,6,7,13-tetrahydro-12H-indolo[2,3-a]pyrrolo[3,4-c]carbazole-12-carboxylate, compound 16. To a solution of compound 15 (0.3 g, 0.68 mmol) in toluene (700 ml) iodine (1.7 g, 6.8 mmol) was added. The resulting mixture was irradiated for 4 h with high-pressure mercury lamp (400W) then the solution was concentrated in vacuo to dryness. The residue was crystallized from ether to afford compound 15 (150 mg, 59%). Product purity was confirmed by ¹H-NMR (400 MHz, DMSO-d6): δ (ppm) 11.7 (s, 1H), 11.61 (s, 1H), 8.92 (d, 1H), 7.78 (d, 1H), 7.27-7.67 (m, 9H), 7.02 (d, 1H), 6.59 (s, 1H), 6.4 (d, 1H), 5.34 (s, 2H), 4.74 (s, 2H), 3.84 (s, 3H), 3.71 (s, 3H), 1.68 (s, 9H).

Step 5: Preparation of 1-(2-chloroethyl)-morpholine free base. 1-(2-Chlorethyl)-morpholine hydrochloride (5.00 g, 26.86 mmol) was dissolved in water (10 ml) and to this solution ether (10 ml) was added. To a vigorously stirred mixture a solution of KOH (1.50 g, 26.80 mmol) in water (10 ml) was added dropwise at rt over 5 min. The organic layer was separated, and the aqueous layer was extracted with ether (2×20 ml). The combined organic extracts were washed with water, brine, dried over sodium sulfate, and evaporated in vacuo to dryness resulting in crude 1-(2-chloroethyl)-morpholine (3.1 g, 78%) as a pale-yellow oil. The product was used in next synthesis without purification.

Step 6: Synthesis of 2-(benzyloxy)-6-(2,4-dimethoxybenzyl)-3-fluoro-12-(2-morpholin-4-ylethyl)-12,13-dihydro-5H-indolo[2,3-a]pyrrolo[3,4-c]carbazole-5,7(6H)-dione and 2-(benzyloxy)-6-(2,4-dimethoxybenzyl)-3-fluoro-13-(2-morpholin-4-ylethyl)-12,13-dihydro-5H-indolo[2,3-a]pyrrolo[3,4-c]carbazole-5,7(6H)-dione, isomers 17A and 17B, respectively. To a solution of compound 5 (0.37 g, 0.5 mmol) in DMF (3 mL) a 60% sodium hydride (0.042 g, 1.0 mmol) was added. The resulting dark mixture was stirred at ambient temperature for 1 h, then 1-(2-chloroethyl)-morpholine (0.15 g, 1.0 mmol) was added. The reaction mixture was stirred at ambient temperature for 16 h, and then the resulting mixture was poured into an ice-cold 10% aq solution of citric acid (20 ml), The resulting mixture was extracted with EtOAc (2×10 mL), the organic layer was washed with water, brine, dried over sodium sulfite, and evaporated in vacuo to dryness. The residue was purified via column chromatography (silica gel, eluent DCM with an EtOAc gradient 0% to 100%) to afford the compound 17A (140 mg, 37%) ¹H-NMR (400 MHz, DMSO-d6): δ (ppm) 12.10 (br, 1H), 8.98 (d, 1H), 8.63 (d, 1H), 7.76 (d, 1H), 7.51-7.65 (m, 4H), 7.46 (t, 2H), 7.27-7.41 (m, 2H), 6.95 (d, 1H), 6.57 (s, 1H), 6.39 (d, 1H), 5.34 (s, 2H), 4.97 (s, 2H), 4.63 (s, 2H), 3.82 (s, 3) 3.69 (s, 3H), 3.29 (s, 4H), 2.44 (s, 2H), 2.32 (s, 4H); and compound 17B (160 mg, 42%) ¹H-NMR (400 MHz, DMSO-d6): δ (ppm) 12.00 (s, 1H), 9.02 (d, 1H), 8.67 (d, 1H), 7.75 (d, 1H), 7.51-7.65 (m, 3H), 7.27-7.50 (m, 5H), 6.99 (d, 1H), 6.58 (s, 1H), 6.41 (d, 1H), 5.35 (s, 2H), 4.90 (s, 2H), 4.67 (m, 2H), 3.83 (s, 3H), 3.71 (s, 3H), 3.28 (s, 4H), 2.75 (m, 2H), 2.34 (s, 4H).

Step 7: Synthesis of 3-fluoro-2-hydroxy-12-(2-morpholin-4-ylethyl)-12,13-dihydro-5H-indolo[2,3-a]pyrrolo[3,4-c]carbazole-5,7(6H)-dione (18). A solution of 17A (0.140 g, 0.19 mmol) in a mixture of anisole/TFA 1:1 (2 ml) was stirred in a microwave reactor at 150° C. for 2 h. The resulting mixture was cooled to rt and diluted with ether (5 ml). The precipitate was filtered, washed with ether, and the resulting solids were dissolved in a mixture of EtOAc/THF 2:1 (10 ml). The solution was washed with 10% aq sodium bicarbonate, water, and brine. The organic solution was dried over sodium sulfate, evaporated in vacuo to dryness and crystallized from ether to afford 18 (70 mg, 89%). Final product purity was confirmed by NMR and HPLC (C18 column, acetonitrile, 5% to 87% over 10 min, retention time 6.19 min). The structure was assigned based on the 2D-NOESY NMR H-H and H-F correlations. ¹H-NMR (400 MHz, DMSO-d6): δ (ppm) 12.00 (hr, 1H), 11.00 (s, 1H), 10.36 (s, 1H), 9.04 (d, 1H), 8.73 (d, 1H), 7.77 (d, 1H), 7.56 (t, 1H), 7.20-7.43 (m, 2H), 4.93 (s, 2H), 3.41 (s, 4H), 2.79 (s, 2H), 2.41 (s, 4H). MS (ESI) m/z 473.3 [MH]+.

Step 8. Synthesis (3-fluoro-2-hydroxy-13-(2-morpholin-4-ylethyl)-12,13-dihydro-5H-indolo[2,3-a]pyrrolo[3,4-c]carbazole-5,7(6H)-dione (19). The same procedure as described in Step 7 was employed using compound 17B to afford the isomeric product compound 19 (70 mg, 81%). Final product purity was confirmed by NMR and HPLC (C18 column, acetonitrile, 5% to 87% over 10 min, retention time 6.06 min). The structure was assigned based on the 2D-NOESY H-H and H-F correlations. ¹H-NMR (400 MHz, DMSO-d6): δ (ppm) 11.95 (br, 1H), 10.98 (s, 1H), 10.35 (br, 1H), 9.06 (d, 1H), 8.70 (d, 1H), 7.8 (d, 1H), 7.58 (t, 1H), 7.18-7.47 (m, 2H), 4.97 (s, 2H), 3.38 (s, 4H), 2.75 (s, 2H), 2.37 (s, 4H) MS (ESI) m/z 473.3 [MH]+.

### Example 5 Synthesis of Compounds 23 and 24 (Arbitrary Assigned Individual Isomers)

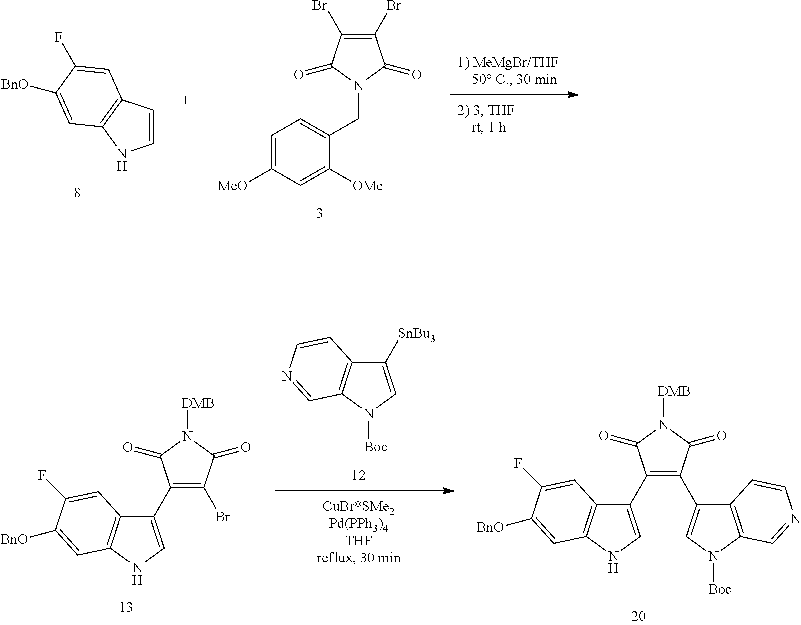

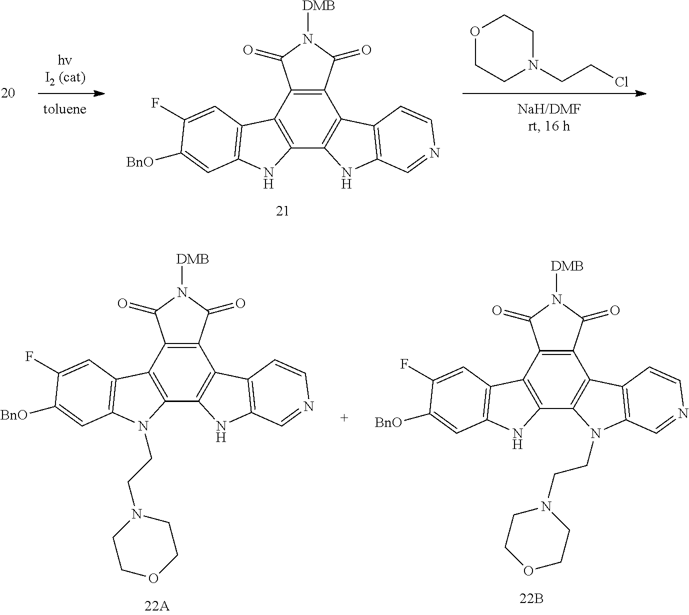

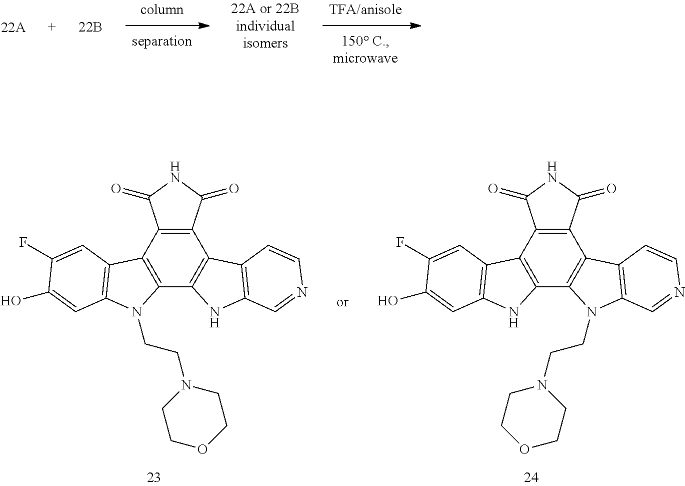 [SMILES: O=C1NC(=O)c2c1c1c3ccncc3[nH]c1c1c2c2cc(F)c(O)cc2n1CCN1CCOCC1 ; O=C1NC(=O)c2c1c1c3cc(F)c(O)cc3[nH]c1c1c2c2ccncc2n1CCN1CCOCC1]

Step 1: Synthesis of 3-(6-(benzyloxy)-5-fluoro-1H-indol-3-yl)-4-bromo-1-(2,4-dimethoxybenzyl)-1H-pyrrole-2,5-dione, compound 3. To a stirred solution of 6-(benzyloxy)-5-fluoro-1H-indole 8 (12 g, 5.2 mmol, see Example 2) in THF (25 ml) a 1.4M methylmagnesium bromide solution in a mixture of THF/toluene 1:3 (3.8 mL, 5.2 mmol) was added dropwise under an argon atmosphere at ambient temperature. The resulting dark solution was stirred at 40-50° C. for 1 h. The reaction mixture was cooled to ambient temperature and a solution of 3,4-dibromo-1-(2,4-dimethoxybenzyl)-1H-pyrrole-2,5-dione 3 (1.0 g, 2.6 mmol) in THF (25 ml) was added dropwise at ambient temperature within 1 h under an argon atmosphere. The reaction mixture was stirred at ambient temperature for 2 h, and then was poured into an ice-cold 10% aq solution of citric acid (200 ml). The resulting mixture was extracted with EtOAc (2×70 ml), the organic layer was washed with water, brine, dried over sodium sulfate, and evaporated in vacuo to dryness. The residue was purified via column chromatography (silica gel, eluent DCM 100% to DCM/EtOAc 9:1) to afford compound 13 (12 g, 86%). Product purity was confirmed by ¹H-NMR (400 MHz, CDCl₃): δ (ppm) 8.71 (br, 1H), 7.96 (d, 1H), 7.86 (d, 1H), 7.43-7.50 (m, 2H), 7.30-7.43 (m, 4H), 7.22 (d, 1H), 6.97 (d, 1H), 6.37-6.54 (A, 1H), 5.18 (s, 2H), 4.80 (s, 2H), 3.84 (s, 3H), 3.79 (s, 3H).

Step 2: Synthesis of tert-butyl 3-(4-(6-benzyloxy)-5-fluoro-1H-indol-3-yl)-1-(2,4-dimethoxybenzyl)-2,5-dioxo-2,5-dihydro-1H-pyrrol-3-yl)-1H-pyrrolo[2,3-c]pyridine-1-carboxylate, compound 20. To a stirred solution of compound 13 (1.0 g, 1.8 mmol) and tert-butyl 3-(tributylstannyl)-1H-pyrrolo[2,3-c]pyridine-1-carboxylate (12)(1.3 g, 2.6 mmol) in THF (50 ml) were added CuBr*SMe₂ (0.55 g, 2.6 mmol) and Pd(PPh₃)₄ (0.04 g, 0.03 mmol), and the resulting mixture was refluxed for 30 min. The reaction mixture was cooled to rt, and then 33% aq NH₄OH was added (2 ml/mmol). The catalyst was removed via filtration through a Celite bed, the Celite layer was washed with EtOAc (2×30 ml). The organic layer was separated, and then was washed with 20% aq sodium carbonate until a blue discoloration has disappeared, then washed with brine, and evaporated in vacuo to dryness. The residue was purified via flash column chromatography (silica gel, eluent DCM 100% to DCM/EtOAc 1:1) to afford compound 20 (0.7 g, 56%). Product purity was confirmed by ¹H-NMR (400 MHz, DMSO-d6): δ (ppm) 11.83 (s, 1H), 9.27 (s, 1H), 8.12 (s, 1H), 8.03 (d, 1H), 7.82 (d, 1H), 7.27-7.47 (m, 5), 7.16 (d, 1H), 7.08 (d, 1H), 6.82 (d, 1H), 6.54-6.63 (m, 2H), 6.48 (dd, 1H), 5.12 (s, 2H), 4.68 (s, 2H), 3.82 (s, 3H), 3.73 (s, 3H), 1.65 (s, 9H). MS (ESI) m/z 703.7 [MH]+.

Step 3: Synthesis of 10-(benzyloxy)-6-(2,4-dimethoxybenzyl)-9-fluoro-12,13-dihydro-5H-pyrido[4′,3′:4,5]pyrrolo[2,3-a]pyrrolo[3,4-c]carbazole-5,7(6H)-dione, compound 21. To a solution of compound 20 (0.2 g, 0.28 mmol) in toluene (700 mL) was added iodine (0.7 g, 2.8 mmol). The resulting mixture was irradiated for 4 h with a high-pressure mercury lamp (400W), then evaporated in vacuo to dryness and recrystallized from ether to afford compound 21 (0.150 mg, 88%) as a yellow solid. Product purity was confirmed by ¹H-NMR (400 MHz, DMSO-d6): δ (ppm) 12.09 (br, 1H), 11.87 (br, 1H), 9.11 (s, 1H), 8.62 (s, 1H), 8.35-8.52 (m, 2H), 730-7.65 (m, 8H), 7.00 (d, 1H), 6.69 (s, 1H), 6.40 (d, 1H), 5.24 (s, 2), 4.68 (s, 2H), 3.84 (s, 3H), 3.71 (s, 3H). MS (ESI) m/z 601.5 [MH]+.

Step 4: Synthesis of compounds 22A and 22B (regioisomers). To a stirred solution of compound 21 (0.200 g, 0.33 mmol) in DMF (1 ml) a 60% sodium hydride (0.026 g, 0.66 mmol) was added. The resulting dark mixture was stirred at ambient temperature for 1 h, then 1-(2-chloroethyl)-morpholine (0.1 g, 0.66 mmol) was added. The reaction mixture was stirred at ambient temperature for 16 h, then poured then was poured into an ice-cold 10% aq solution of citric acid (20 ml). The resulting mixture was extracted with EtOAc/THF (2×10 ml), the organic layer we washed with water, brine, dried over sodium sulfate, and concentrated in vacuo to dryness. The residue was purified by column chromatography (silica gel, eluent DCM 100% to DCM+2.5 vol % MeOH) to afford individual (arbitrary structure assignments) isomers 22A (40 mg, 16%) and 22B (40 mg, 16%). Final products purity was confirmed by ¹H-NMR (22A)(400 MHz, CDCl₃): δ (ppm) 9.30 (s, 1H), 8.95 (s, 1H), 8.76 (s, 1H), 8.46 (s, 1H) 7.29-7.66 (m, 8H), 7.11-7.24 (m, 1H), 6.89-7.05 (m, 1H), 6.24-6.57 (m, 2H), 5.31 (s, 2H), 4.74 (s, 4H), 3.85 (s, 3H), 3.74 (s, 3H), 3.66 (s, 4H), 3.05 (s, 2H), 2.67 (s, 4H); and (22B) (400 MHz, CDCl₃): δ (ppm) 12.43 (br, 1H), 8.40-9.11 (m, 4H), 7.30-7.63 (m, 8H), 7.00-7.20 (m, 2), 6.30-6.59 (m, 2H), 5.32 (s, 2H), 4.82 (s, 4H), 3.89 (s, 3H), 3.75 (s, 3H), 3.68 (s, 4H), 3.17 (s, 2H), 2.68 (s, 4H). MS (ESI) m/z 714.3 [MH]+.

Step 5: Synthesis of compound 23. A solution of the 22A isomer (0.080 g, 0.1 mmol) in mixture anisole/TFA 1:1 (2 ml) was stirred in a microwave reactor at 150° C. for 2 h. The resulting mixture was cooled to ambient temperature and diluted with ether (5 ml). The precipitate was filtered, washed with ether to afford a TFA salt of 23 (0.050 g, 66%). The solids were dissolved in a mixture of EtOAc/THF 2:1 (10 mL) and washed with 10% aq sodium bicarbonate, water, and brine. The organic solution was dried over sodium sulfate, evaporated in vacuo to dryness, and the residue was crystallized in ether to afford a free base form of 23 (9 mg, 17%). Final product purity was confirmed by NMR and HPLC (C18 column, 5% to 87% acetonitrile over 10 min, retention time 4.61 min). ¹H-NMR (400 MHz, DMSO-d6): δ (ppm) 12.07 (br, 1H), 11.09 (br, 1H), 10.41 (br, 1H), 9.24 (m, 1H), 8.81 (m, 1H), 9.48 (d, 1H), 7.34 (m, 1H), 5.00 (s, 2H), 3.37 (s, 4H), 2.79 (s, 2H), 2.33 (s, 4H). MS (ESI) m/z 474.3 [M+H]+.

Step 6. Synthesis of compound 24. The same procedure as described in Step 5 was employed using the 22B to afford the free base form of isomeric product compound 24 (8 mg, 16%). Final product purity was confirmed by NMR and HPLC (C18 column, 5% to 87% acetonitrile over 10 min, retention time 4.44 min). ¹H-NMR (400 MH, DMSO-d6): δ (ppm) 12.33 (br, 1H), 11.06 (br, 1H), 10.46 (br, 1H), 9.14 (s, 1H), 8.81 (m, 1H), 8.70 (m, 1H), 8.49 (m, 1H), 7.33 (m, 1H), 4.90 (s, 2H), 3.39 (m, 4H), 2.75 (s, 2H), 2.36 (m, 4H). MS (ESI) m/z 474.3 [MH]+.

### Example 6. Synthesis of 2,10-dihydroxy-12-(2-morpholinoethyl)-12,13-dihydro-5H-indolo[2,3-a]pyrrolo[3,4-c]carbazole-5,7(6H)-dione, compound 32

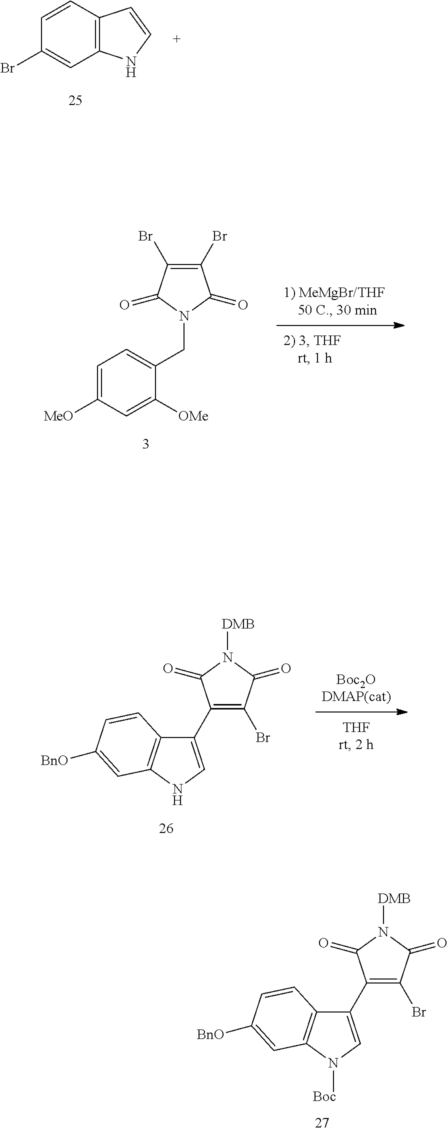

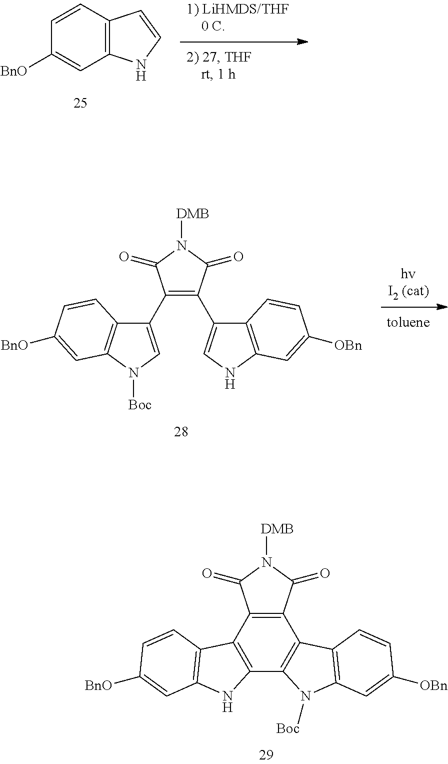

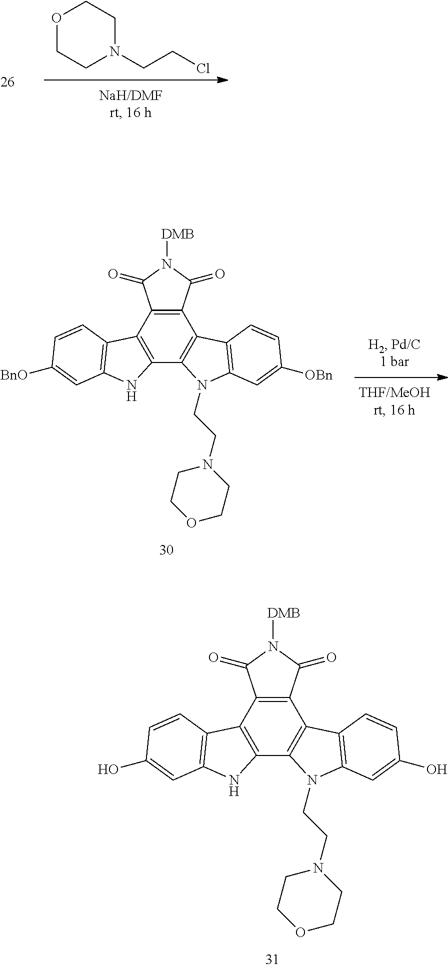

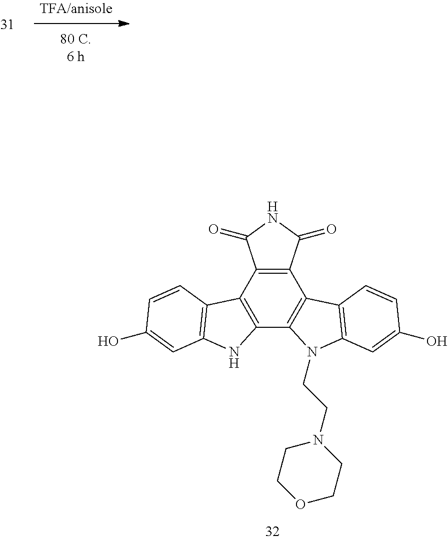 [SMILES: O=C1NC(=O)c2c1c1c3ccc(O)cc3[nH]c1c1c2c2ccc(O)cc2n1CCN1CCOCC1]

Step 1: Synthesis of 3-(6-(benzyloxy)-1H-indol-3-yl)-4-bromo-1-(2,4-dimethoxybenzyl)-1H-pyrrole-2,5-dione, compound 26, To a stirred solution of 6-(benzyloxy)-1H-indole (25) (2.20 g, 9.87 mmol) in THF (25 ml) a 1.4 M methylmagnesium bromide solution in THF/toluene 1:3 (7.0 ml, 9.87 mmol) was added dropwise under an argon atmosphere at rt. The resulting dark solution was stirred at 50-60° C. for 1 h. The reaction mixture was cooled to rt and a solution of dibromomaleimide 2 (2.00 g, 4.93 mmol) in THF (25 ml) was added dropwise at rt within t h under an argon atmosphere. The reaction mixture was stirred at rt for 1 h, then was poured into an ice-cold 10% aq solution of citric acid (200 ml). The resulting mixture was extracted with EtOAc (2×70 ml), an organic layer was washed with water, brine, dried over sodium sulfate, and concentrated in vacuo to dryness. The residue was purified by column chromatography (silica gel, eluent DCM/CCl₄ 1:1 to 100% DCM) to afford compound 26 (1.80 g, 66%) as a red-brownish solid, Final product purity was confirmed by ¹H-NMR (400 MHz, DMSO-d6): δ (ppm) 11.96 (br, 1H), 9.00 (d, 1H), 7.83 (d, 1H), 7.46-7.48 (m, 2H), 7.40 (t, 2H), 7.30-7.35 (m, 1H), 7.02-7.07 (m, 2H), 6.89 (dd, 1H), 6.56-6.57 (m, 1H), 6.46 (dd, 1H), 5.15 (s, 2H), 4.61 (s, 2H), 3.81 (s, 3H), 3.70 (s, 3H). MS (ESI) m/z 549.5 [MH]+.

Step 2: Synthesis of tert-butyl 6-(benzyloxy)-3-[4-bromo-1-(2,4-dimethoxybenzyl)-2,5-dioxo-2,5-dihydro-1H-pyrrol-3-yl]-1H-indole-1-carboxylate, compound 27. To a stirred solution of compound 26 (1.80 g, 3.28 mmol) and DMAP (0.02 g, 0.16 mmol) in THF (20 ml) a solution of Boc-anhydride (0.79 g, 3.61 mmol) in THF (5 ml) was added at rt under an argon atmosphere. The resulting solution was stirred at rt for 2 h, then was concentrated in vacuo to dryness. The residue was purified by column chromatography (silica gel, eluent DCM/CCl₄ 1:2 to 100% DCM) to afford compound 27 (1.55 g, 73%) as a yellow solid. Product purity was confirmed by ¹H-NMR (400 MHz, DMSO-d6): δ (ppm) 8.02 (s, 1H), 7.77 (br, 1H), 7.70 (d, 1H) 7.47-7.49 (m, 2M), 7.49 (t, 2H), 7.31-7.35 (m, 1H), 7.09 (t, 2H), 6.56 (s, 1H), 6.46 (dd, 1H), 5.19 (s, 2H), 4.62 (s, 2H), 3.82 (s, 3H), 3.73 (s, 3H), 1.64 (s, 9H).

Step 3: Synthesis of tert-butyl 6-(benzyloxy)-3-[4-[6-(benzyloxy)-1H-indol-3-yl]-1-(2,4-dimethoxybenzyl)-2,5-dioxo-2,5-dihydro-1H-pyrrol-3-yl]-1H-indole-1-carboxylate, compound 28, To a stirred solution of HMDS (1.11 g, 6.90 mmol) in THF (50 ml) a 2.5M solution of butyllithium in THF (2.8 ml, 6.90 mmol) was added dropwise at 0° C. under an argon atmosphere. The resulting solution was stirred for 30 min at 0° C., then cooled down to −20° C. and a solution of 6-(benzyloxy)-1H-indole (0.62 g, 2.78 mmol) in THF (5 ml) was added dropwise. The reaction mixture was stirred at −20° C. for 45 min. Then a solution of compound 27 (1.50 g, 2.30 mmol) in THY (20 ml) was added dropwise at −20° C. within 45 min under an argon atmosphere. The resulting mixture was stirred at −20° C. for 45 min and then for 1 h at 0° C., then poured into an ice-cold 10% aq solution of citric acid (200 ml). The resulting mixture was extracted with EtOAc (2×70 ml), an organic layer was washed with water, brine, dried over sodium sulfite, and concentrated in vacuo to dryness. The residue was purified by column chromatography (silica gel, eluent 100% DCM to DCM/ether 5:1) to afford compound 28 (1.10 g, 61%) as red-brownish solid. Product purity was confirmed by ¹H-NMR (400 MHz, DMSO-d6): 11.68 (br, 1H), 7.81-7.82 (m, 1H), 7.76-7.78 (m, 1H), 7.71 (br, 1H), 7.30-7.41 (m, 10H), 7.04 (dd, 1H), 6.97 (br, 1H), 6.47 (t, 2H), 6.57-6.63 (m, 2H), 6.47 (dd, 2H), 6.05 (d, 4), 4.66 (s, 2), 3.80 (s, 3H), 3.71 (s, 3H), 1.60 (s, 9H).

Step 4: Synthesis of tert-butyl 2,10-bis(benzyloxy)-6-(2,4-dimethoxybenzyl)-5,7-dioxo-5,6,7,13-tetrahydro-12H-indolo[2,3-a]pyrrolo[3,4-c]carbazole-12-carboxylate, compound 29. To a solution of compound 28 (0.350 g, 0.443 mmol) in toluene (700 ml) a catalytic amount of iodine was added. The resulting mixture was irradiated for 2 h with a high-pressure mercury lamp (400W), then evaporated in vacuo to dryness and recrystallized from ether to afford 0.250 g 29 as a dark-green solid of the crude product. The crude product was dissolved in DCM, and purified by column chromatography (silica gel, eluent DCM/ether 5:1) to afford compound 29 (0.160 g, 47%) as a yellow solid. Product purity was confirmed by ¹H-NMR (400 MHz, DMSO-d6) 11.17 (br, 1H), 8.99 (d, 1H), 8.80 (d, 1H), 7.69 (1H), 7.36-7.52 (m, 11H), 7.11 (d, 1H), 6.96 (t, 2H), 6.57 (br, 1H), 6.39 (d, 1H), 5.19 (d, 4H), 4.65 (s, 2H), 3.80 (s, 3H), 3.70 (s, 3H), 1.79 (s, 9H).

Step 5: Synthesis of 2,10-bis(benzyloxy)-6-(2,4-dimethoxybenzyl)-12-(2-morpholinoethyl)-12,13-dihydro-5H-indolo[2,3-a]pyrrolo[3,4-c]carbazole-5,7(6H)-dione, compound 7, To a solution of compound 29 (0.240 g, 0.304 mmol) in DMF (1 ml) a 60% sodium hydride (0.024 g, 0.608 mmol) was added. The resulting dark mixture was stirred at rt for 1 h, then 1-(2-chloroethyl)-morpholine (0.091 g, 0.608 mmol) was added. The reaction mixture was stirred at rt for 16 h, then was poured into an ice-cold 10% aq solution of citric acid (20 ml). The resulting mixture was extracted with EtOAc (2×10 ml), an organic layer was washed with water, brine, dried over sodium sulfate, and concentrated in vacuo to dryness. The residue was purified by column chromatography (silica gel, eluent DCM/ether 5:1) to afford compound 30 (0.130 g, 54%) as a yellow solid, Final product purity was confirmed by NMR and HPLC (C18 column, acetonitrile, 40% to 87% over 10 min, retention time 7.28 min). ¹H-NMR (400 MHz, DMSO-d6Y): δ (ppm) 11.91 (br, 1H), 828-8.91 (m, 2H), 7.52-7.54 (m, 4H), 7.35-7.45 (m, 7H), 7.28 (br, 1H), 7.02-7.06 (m, 3H), 6.59 (br, 1H), 6.43 (d, 1H), 5.28 (br, 4H), 4.91 (br, 2H), 4.76 (br, 2R), 3.83 (s, 3H), 3.74 (s, 3H), 3.29 (br, 4H), 2.72 (br, 28), 2.34 (br, 4H). MS (ESI) m/z 801.5 [MH]+.

Step 6: Synthesis of Synthesis of 6-(2,4-dimethoxybenzyl)-2,10-dihydroxy-12-(2-morpholinoethyl)-12,13-dihydro-5H-indol[2,3-a]pyrrolo[3,4-c]carbazole-5,7(6H)-dione, compound 31. To a solution of compound 30 (0.130 g, 0.162 mmol) in a 1:1 THF/MeOH mixture (20 ml) a catalytic amount of 10% palladium on carbon (10 mg) was added. The resulting mixture was stirred under a hydrogen atmosphere at rt for 16 h. The solution was filtered to remove the catalyst, and filter was washed with THF/MeOH. The filtrate was concentrated in vacuo to dryness and crystallized from ether to afford a crude product 31 (0.080 g, 80%) as a yellow solid. Final product purity was confirmed by NMR and HPLC (C18 column, acetonitrile 5% to 87% over 10 min, retention time 7.07 min). ¹H-NMR (400 MHz, DMSO-d6). δ (ppm) 11.75 (br, 1H), 9.83 (s, 1H), 9.738 (s, 1H), 8.80 (t 2H), 7.11 (d, 1H), 7.00-7.07 (m, 2H), 6.78-6.83 (m, 2H), 6.59 (d, 1H), 6.43 (dd, 114), 4.83 (t, 2H), 4.7 (s, 2H), 3.83 (s, 3), 3.71 (s, 3H), 3.38 (m, 4H), 2.75 (t, 2H), 2.40 (m, 4H). MS (ESI) m/z 621.3 [MH]+.

Step 7: Synthesis of 2,10-dihydroxy-12-(2-morpholinoethyl)-12,13-dihydro-5H-indolo[2,3-a]pyrrolo[3,4-c]carbazole-5,7(6H)-dione, compound 32. A solution of 8 (0.080 g, 0.129 mmol) in the anisole/TFA 1:1 mixture (4 ml) was stirred under an argon atmosphere at 70-80° C. for 5 h. The resulting mixture was cooled down to rt and diluted with ether (5 mi). The precipitate was filtered, washed with ether affording 32 (0.050 g, 66%) as the TFA salt. The solid residue was dissolved in the EtOAc/THF 2:1 mixture (10 ml) and washed with 10% aq solution of sodium hydrogen carbonate, water, and brine. The solution was dried over sodium sulfate, concentrated in vacuo to dryness, and recrystallized from ether to afford a free base form of 32 (0.030 g, 40%). Final product purity was confirmed by NMR and HPLC (C18 column, acetonitrile 5% to 87% over 10 min, retention time 5.50 min). ¹H-NMR (400 MHz, DMSO-d6): δ (ppm) 11.56 (br, 1H), 10.89 (br, 1H), 9.92 (br, 1H), 9.82 (br, 1H), 8.80-8.86 (m, 2H), 7.12-7.14 (m, 2H), 6.89 (d, 1H), 6.81 (d, 1H), 5.10 (br, 2H), 3.70 (br, 4H) 2.40-2.53 (m, 6H). MS (ESI) m/z 471.3 [MH]+.

### Example 7. Synthesis of 3,9-difluoro-2,10-dihydroxy-12-(2-morpholinoethyl)-12,13-dihydro-5H-indolo[2,3-a]pyrrolo[3,4-c]carbazole-5,7(6H)-dione, compound 37

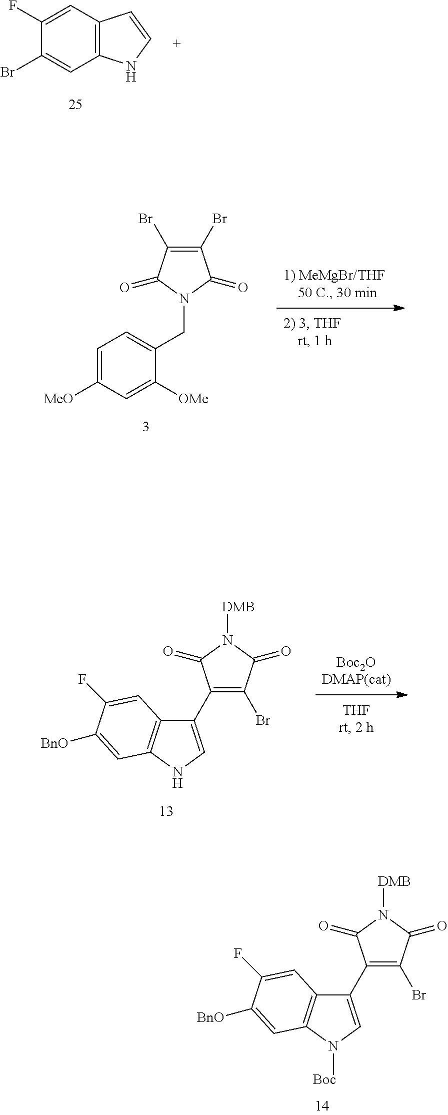

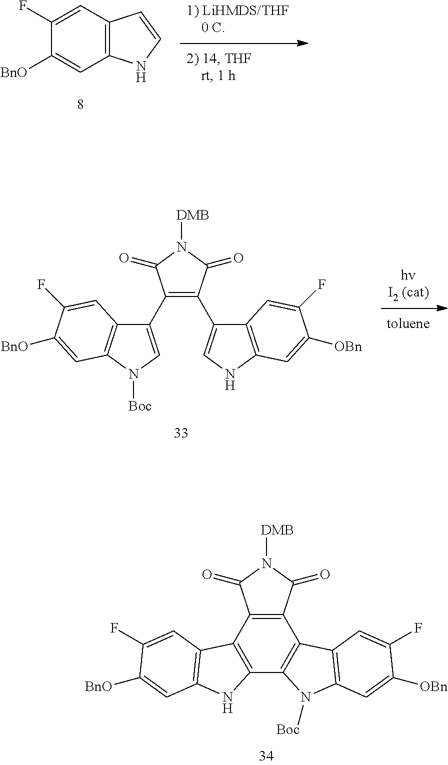

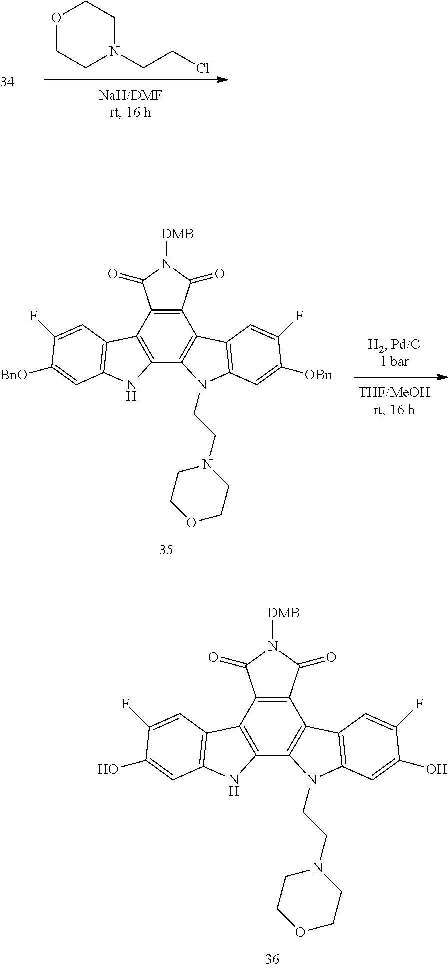

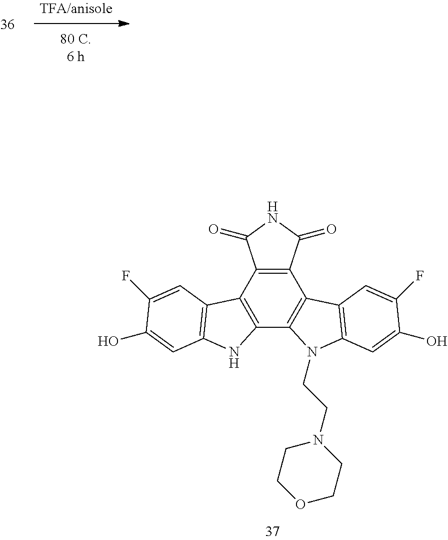 [SMILES: O=C1NC(=O)c2c1c1c3cc(F)c(O)cc3[nH]c1c1c2c2cc(F)c(O)cc2n1CCN1CCOCC1]

Steps 1-7. The same procedure employed in Example 6 was used to produce the title compound 37, with the exception that 6-(benzyloxy)-5-fluoro-1H-indole (compound 9 in Example 2) was used in Steps 1 and 3 in place of 6-(benzyloxy)-1H-indole. This procedure afforded the free base form of compound 37 (0.030 g, 40%) as a yellow solid. Final product purity was confirmed by NMR and HPLC (C18 column, acetonitrile 5% to 87% over 10 min, retention time 6.02 min), 1′H-NMR (400 MHz, DMSO-d6): δ (ppm) 11.82 (br, 1H), 10.95 (s, 1H), 10.29 (s, 2H), 8.67 (dd, 2), 7.28 (dd, 2H), 4.4 (t, 2H), 334 (m, 4H), 2.71 (t, 2H), 2.54 (m, 2H), 237 (m, 2H), MS (ESI) m/z 507.4 [MH]+.

### Example 8. Synthesis of Compounds 41 and 42

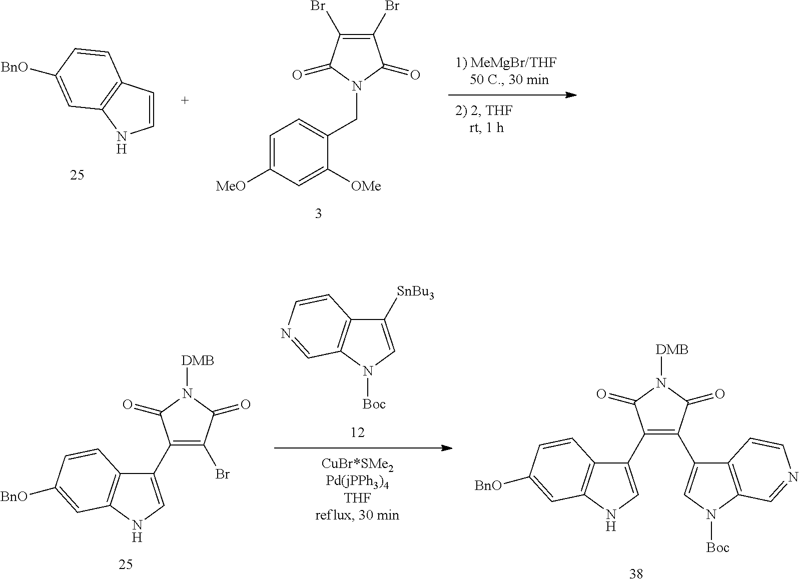

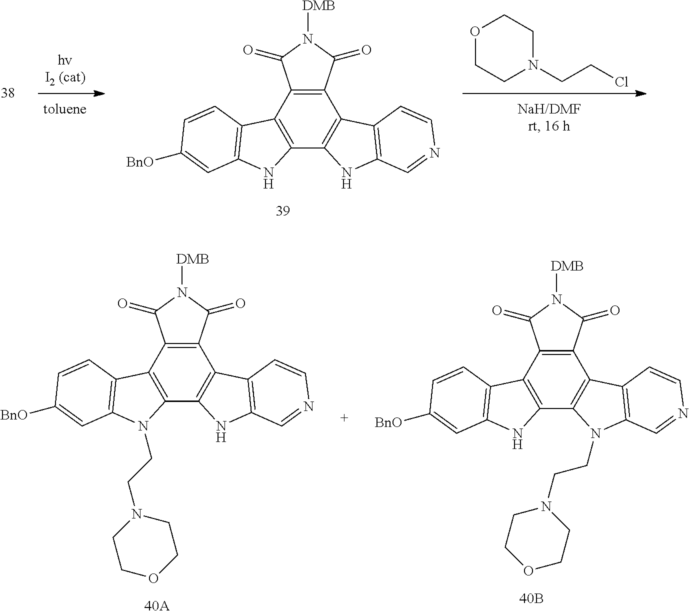

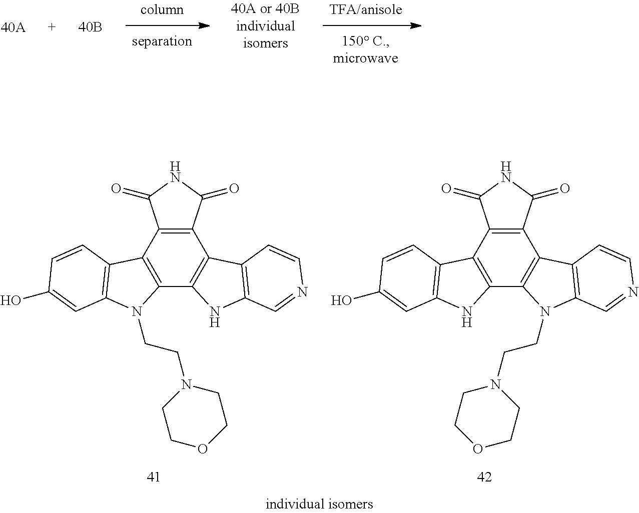

Step 1: Synthesis of 3-[6-(benzyloxy)1H-indol-3-yl]-4-bromo-1-(2,4-dimethoxybenzyl)-1H-pyrrole-2,5-dione, compound 26. To a stirred solution of 6-(benzyloxy)-1H-indole 25 (3.3 g, 14.9 mmol) in THF (50 mL) a 1.4M methylmagnesium bromide solution in a mixture of THF/toluene 1:3 (10.6 mL, 14.9 mmol) was added dropwise under an argon atmosphere at ambient temperature. The resulting dark solution was stirred at 40-50° C. for 1 h. The reaction mixture was cooled to ambient temperature and a solution of 3,4-dibromo-1-(2,4-dimethoxybenzyl)-1H-pyrrole-2,5-dione 3 (3.0 g, 7.5 mmol) in THF (25 mL) was added dropwise at ambient temperature for a 1 h under Ar. The reaction mixture was stirred at ambient temperature within 1 h under an argon atmosphere. The reaction mixture was stirred at ambient temperature for 2 h, and then was poured into an ice-cold 10% aq solution of citric acid (200 ml). The resulting mixture was extracted with EtOAc (2×100 ml), the organic layer was washed with water, brine, dried over sodium sulfate and evaporated in vacuo to dryness. The residue was purified via column chromatography (silica gel, eluent DCM 100% to DCM/EtOAc 9:1) to afford compound 26 (3.2 g, 75%). Product purity was confirmed by ¹H-NMR (400 MHz, DMSO-d6): δ (ppm) L96 (s, 1H), 7.99 (s, 1H), 7.84 (d, 1H), 7.28-7.55 (m, 5H, 6.99-7.11 (m, 2H), 6.90 (dd, 1H), 6.56 (s, 1H), 6.47 (dd, 1H), 5.15 (s, 2H), 4.61 (s, 2H), 3.79 (s, 3H), 3.73 (s, 3H).

Step 2: Synthesis of tert-butyl 3-[4-[6-(benzyloxy)-1H-indol-3-yl]-1-(2,4-dimethoxybenzyl)-2,5-dioxo-2,5-dihydro-1H-pyrrol-3-yl]-1H-pyrrolo[2,3-c]pyridine-1-carboxylate, compound 38. To a stirred solution of compound 26 (0.6 g, 1.1 mmol) and tert-butyl 3-tributylstannyl)-1H-pyrrolo[2,3-c]pyridine-1-carboxylate (12)(0.72 g, 1.4 mmol) in THF (50 ml) were added CuBr*SMe₂ (0.29 g, 1.4 mmol) and Pd(PPh₃)₄ (0.025 g, 0.022 mmol), and the resulting mixture was refluxed for 30 min. The reaction mixture was cooled to rt, and then 33% aq NH₄OH was added (3.6 ml). The catalyst was removed via filtration through a Celite bed, the Celite layer was washed with EtOAc (2×30 ml). The organic layer was separated, and then was washed with 20% aq sodium carbonate until a blue discoloration has disappeared, then washed with brine, and evaporated in vacuo to dryness. The residue was purified via flash column chromatography (silica gel, eluent DCM 100% to DCM/EtOAc 1:1) to afford compound 38 (0.45 g, 60%). Product purity was confirmed by ¹H-NMR (400 MHz, DMSO-d6): δ (ppm) 11.76 (s, 1H), 9.25 (s, 1H), 0.01 (s, 1H), 8.06 (d, 1H) 7.83 (d, 1H), 7.21-7.47 (m, 6H), 7.06 (d, 1H), 6.98 (s, 1H), 6.82 (d, 1H), 6.70 (d, 2H), 6.57 (s, 1H), 6.39-6.51 (m, 2), 5.02 (s, 2H), 4.67 (s, 2H), 3.81 (s, 3H), 3.73 (s, 3H), 1.64 (s, 9H). MS (ESI) m/z 685.7 [MH]+.

Step 3: Synthesis of 10-(benzyloxy)-6-(2,4-dimethoxybenzyl)-12,13-dihydro-5H-pyrido[4′,3′:4,5]pyrrolo[2,3-a]pyrrolo[3,4-c]carbazole-5,7(6H)-dione, compound 39. To a solution of compound 38 (0.15 g, 0.22 mmol) in toluene (700 mL) was added iodine (0.7 g, 2.8 mmol). The resulting mixture was irradiated for 4 h with a high-pressure mercury lamp (400W), then evaporated in vacuo to dryness and recrystallized from ether to afford compound 39 (0.12 g, 94%) as a yellow solid. Product purity was confirmed by ¹H-NMR (400 MHz, DMSO-d6): δ (ppm) 13.20 (br.s. 1H), 12.56 (s, 1H), 9.22 (s, 1H), 8.70 (d, 1H), 8.50 (d, 1H), 7.50-7.65 (m, 2H), 7.32-7.49 (m, 3H), 7.29 (s, 1H), 7.01 (t, 1H), 6.57 (s, 1H), 6.26 (d, 1H), 4.70 (c, 2H), 4.68 (s, 2H), 3.83 (s, 3H), 3.70 (s, 3H). MS (ESI) m/z 583.3 [MH]+.

Step 4: Synthesis of compound 40A and 40B (individual isomers). To a stirred solution of compound 39 (0.20 g, 0.34 mmol) in DMF (5 ml) a 60% sodium hydride (0.04 g, 0.96 mmol) was added. The resulting dark mixture was stirred at ambient temperature for 1 h, then 1-(2-chloroethyl)-morpholine (08 g, 0.51 mmol) was added. The reaction mixture was stirred at ambient temperature for 16 h, then was poured into an ice-cold 10% aq solution of citric acid (20 ml). The resulting mixture was extracted with EtOAc/MF (2×10 ml), the organic layer was washed with water, brine, dried over sodium sulfate, and concentrated in vacuo to dryness. The residue was purified by column chromatography (silica gel, eluent DCM 100% to DCM+2.5 vol % MeOH) to afford individual regioisomers 40A (65 mg, 27%) and 40B (25 mg, 10%). Final products purity was confirmed by ¹H-NMR (40A) (400 MHz, CDCl₃): δ (ppm) 9.19 (s, 1H), 8.85-9.04 (m, 2H), 8.50 (s, 1H), 7.48-7.60 (m, 2H), 7.34-7.48 (m, 3H), 7.17 (d, 1H), 7.02 (d, 1H), 6.83 (s, 1H), 6.38 (d, 1H), 5.19 (s, 2H), 4.76 (s, 2H), 4.47-4.65 (m, 2H), 3.84 (s, 3H), 3.74 (s, 3H), 3.66 (br, 4H), 2.98 (br, 2H), 2.59 (br, 4H); (40B)(400 MHz, CDCl₃): δ (ppm) 12.27 (br, 1H), 8.80-9.10 (m, 3H), 8.56 (s, 1H), 7.48-7.58 (m, 2H), 7.32-7.49 (m, 3H), 7.0-7.21 (m, 3H), 6.44 (d, 1H), 6.37 (dd, 1H), 5.23 (s, 2H), 4.68 (br 4H), 3.84 (s, 3), 3.74 (s, 3H), 3.40 (br, 4H), 3.01-3.22 (m, 2H), 2.56-2.8 (m, 4H). MS (ESI) m/z 696.3 [MH]+.

Step 5: Synthesis of compound 41. A solution of the 40A (0.065 g, 0.095 mmol) in mixture anisole/TFA 1:1 (2 ml) was stirred in a microwave reactor at 150° C. for 2 h. The resulting mixture was cooled to ambient temperature and diluted with ether (5 ml). The precipitate was filtered, washed with ether to afford a TFA salt of compound 41 (0.07 g). The solids were dissolved in a mixture of EtOAc/THF 2:1 (10 mL) and washed with 10% aq sodium bicarbonate, water, and brine. The organic solution was dried over sodium sulfate, evaporated in vacuo to dryness, and the residue was crystallized in ether to afford a free base form of 41 (40 mg, 97%). ¹H-NMR (400 MHz, DMSO-d6): δ (ppm) 11.06 (br, 1H), 9.93 (br, 1H), 9.15 (s, 1H), 8.82-8.92 (m, 2H), 8.51 (s, 1H), 7.11 (s, 1H), 6.89 (d, 1H), 4.92 (br, 2H), 3.76 (s, 4H), 2.11 (br, 2H), 2.37 (br, 4H). The free base sample was subjected to a preparative HPLC with TFA-containing eluents. The product fraction was evaporated in vacuo to dryness resulting in a TFA salt of 41 (21 mg, 31%). Final product purity was confirmed by NMR and HPLC(C18 column, 5% to 87% acetonitrile over 10 min, retention time 4.33 min). ¹H-NMR (400 MHz, DMSO-d6): δ (ppm) 11.29 (s, 1H), 10.17 (br, 1H), 9.33 (s, 1H), 9.20 (d, 1H), 8.89 (d, 1H), 8.68 (s, 1H), 7.22 (s, 1H), 6.98 (br, 1H), 5.14 (br, 4H), 1.23 (br, 2H). MS (ESI) m/z 456.5 [M+H]+.

Step 6. Synthesis of compound 42. The same procedure as described in Step 5 was employed using compound 40B to afford the TFA salt of product compound 42 (14 mg, 21%). Final product purity was confirmed by NMR and HPLC (C18 column, 5% to 87% acetonitrile over 10 min, retention time 4.41 min). ¹H-NMR (400 MHz, CD₃OD): δ (ppm) 9.33 (s, 1H), 9.23 (d, 1H), 8.26 (d, 1H), 6.99 (s, 1H), 6.54 (d, 1H), 4.95 (br, 2), 3.76 (br, 4H), 3.24 (br, 2H), 2.94 (br, 4H). MS (ESI) m/z 456.5 [MH]+.

### Example 9. Synthesis of tert-butyl 6-(2,4-dimethoxybenzyl)-3,9-difluoro-5,7-dioxo-5,6,7,13-tetrahydro-12H-indolo[2,3-a]pyrrolo[3,4-c]carbazole-12-carboxylate, compound 47

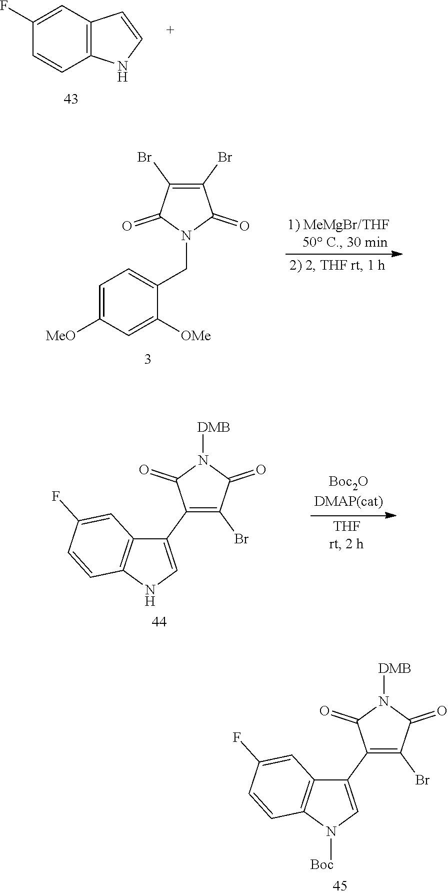

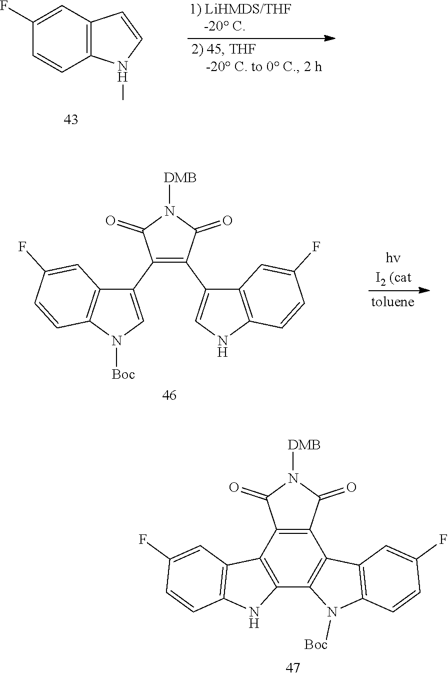

Step 1: Synthesis of 3-bromo-1-(2,4-dimethoxybenzyl)-4(5-fluoro-1H-indol-3-yl)-1H-pyrrole-2,5-dione, compound 44. To a stirred solution of 5-fluoro-1H-indole 43 (2.5 g, 185 mmol, Sigma Aldrich, St. Louis, Mo.) in THY (20 mL) a 1 AM methylmagnesium bromide solution in THF/toluene 1:3 (13.2 ml, 18.5 mmol) was added dropwise under an argon atmosphere at rt. The resulting dark solution was stirred at 50-60° C. for 1 h. The reaction mixture was cooled to rt and a solution of dibromomaleimide 3 (2.5 g, 6.2 mmol) in THF (10 ml) was added dropwise at rt within 1 h under an argon atmosphere. The reaction mixture was stirred at rt for 1 h, then was poured into an ice-cold 10% aq solution of citric acid (100 ml). The resulting mixture was extracted with EtOAc (2×50 ml), an organic layer was washed with water, brine, dried over sodium sulfate, and concentrated in vacuo to dryness. The residue was purified by column chromatography (silica gel, eluent 100% DCM to DCM/EtOAc 9:1) to afford compound 44 (2.8 g, 99%), which was used without further purification.

Step 2: Synthesis of tert-butyl 3-[4-bromo-1-(2,4-dimethoxybenzyl)-2,5-dioxo-2,5-dihydro-1H-pyrrol-3-yl]-5-fluoro-1H-indole-1-carboxylate, compound 45. To a stirred solution of compound 44 (2.8 g, 6.1 mmol) and DMAP (0.035 g, 03 mmol) in THF (20 ml) a solution of Boc-anhydride (1.3 g, 6.1 mmol) in THF (5 ml) was added at it under an argon atmosphere. The resulting solution was stirred at rt for 2 h, then was concentrated in vacuo to dryness. The residue was purified by column chromatography (silica gel, eluent eluent 100% DCM to DCM/EtOAc 9:1) to afford compound 45 (3.0 g, 88%). Product purity was confirmed by ¹H-NMR (400 MHz, DMSO-d6): δ (ppm) 8.90 (s, 1H), 8.14 (dd, 1H), 7.57 (dd, 1H), 7.24-736 (m, 1H), 7.12 (d, 1H), 6.57 (d, 1H), 6.46 (dd, 1H), 4.63 (s, 2H), 3.80 (s, 3H), 3.74 (s, 3H), 1.65 (s, 9H).

Step 3: Synthesis of tert-butyl 3-[1-(2,4-dimethoxybenzyl)-4-(5-fluoro-1H-indol-3-yl)-2,5-dioxo-2,5-dihydro-1H-pyrrol-3-yl]-5-fluoro-1H-indole-1-carboxylate, compound 46. To a stirred solution of HMDS (3.2 g, 16.0 mmol) in THF (50 ml) a 2.5M solution of butyllithium in THF (6.4 ml, 16.0 mmol) was added dropwise at 0° C. under an argon atmosphere. The resulting solution was stirred for 30 min at 0° C., then cooled down to −20° C. and a solution of 5-fluoro-1H-indole 43 (0.9 g, 6.0 mmol) in THF (5 ml) was added dropwise. The reaction mixture was stirred at −20° C. for 45 min. Then a solution of compound 45 (3.0 g, 5.0 mmol) in TH (30 ml) was added dropwise at −20° C. within 45 min under an argon atmosphere. The resulting mixture was stirred at −20° C. for 45 min and then for 1 h at 0° C., then poured into an ice-cold 10% aq solution of citric acid (100 ml). The resulting mixture was extracted with EtOAc (2×100 ml), an organic layer was washed with water, brine, dried over sodium sulfate, and concentrated in vacuo to dryness. The residue was purified by column chromatography (silica gel, eluent 100% DCM to DCM/EtOAc 7:3) to afford compound 46 (2.3 g, 85%).

Step 4: Synthesis of tert-butyl 6-(2,4-dimethoxybenzyl)-3,9-difluoro-5,7-dioxo-5,6,7,13-tetrahydro-12H-indolo[2,3-a]pyrrolo[3,4-c]carbazole-12-carboxylate, compound 47. To a solution of compound 46 (0.50 g, 8 mmol) in toluene (700 ml) a catalytic amount of iodine was added. The resulting mixture was irradiated for 2 h with a high-pressure mercury lamp (400W), then evaporated in vacuo to dryness and recrystallized from ether to afford compound 47 (0.32 g, 64%). Product purity was confirmed by ¹H-NMR (400 MHz, DMSO-d6): 11.72 (s, 1H), 8.51 (dd, 1H), 7.76 (dd, 1H), 7.30-7.47 (m, 1H), 6.96 (d, 1H), 6.57 (s, 1H), 6.40 (d, 1H), 4.62 (s, 2H), 3.84 (s, 3H), 3.71 (a, 3H), 1.67 (s, 9H).

### Example 10. Synthesis of 3,9-difluoro-12-(2-morpholin-4-ylethyl)-12,13-dihydro-5H-indolo[2,3-a]pyrrolo[3,4-c]carbazole-5,7(6H)-dione, compound 49

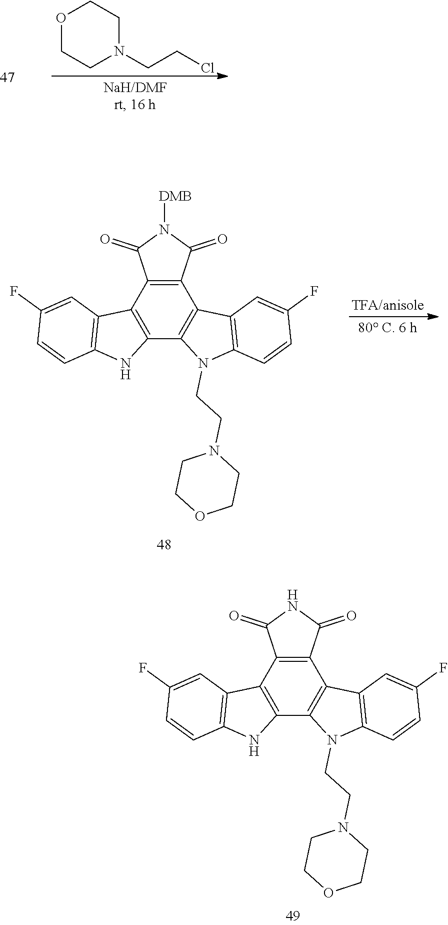

Step 5: Synthesis of 6-(2,4-dimethoxybenzyl)-3,9-difluoro-12-(2-morpholin-4-ylethyl)-12,13-dihydro-5H-indolo[2,3-a]pyrrolo[3,4-c]carbazole-5,7(6H)-dione, compound 48. To a solution of compound 47 (0.2 g, 0.4 mmol) in DMF (5 ml) a 60% sodium hydride (0.047 g, 12 mmol) was added. The resulting dark mixture was stirred at rt for 1 h, then 1-(2-chloroethyl)-morpholine (0.12 g, 0.8 mmol) was added. The reaction mixture was stirred at rt for 16 h, then was poured into an ice-cold 10% aq solution of citric acid (20 ml), The resulting mixture was extracted with EtOAc (2×10 ml), an organic layer was washed with water, brine, dried over sodium sulfate, and concentrated in vacuo to dryness. The residue was purified by column chromatography (silica gel, eluent 100% DCM to 100% EtOAc) to afford compound 4g (0.1 g, 50%).

Step 6: Synthesis of 3,9-difluoro-12-(2-morpholin-4-ylethyl)-12,13-dihydro-5H-indolo[2,3-a]pyrrolo[3,4-c]carbazole-5,7(6H)-dione, compound 49. To a solution of compound 48 (0.1 g, 0.16 mmol) in the anisole/TFA 1:1 mixture (2 ml) was stirred in a microwave reactor at 130° C. for 5 min. The resulting mixture was cooled down to rt and diluted with ether (5 ml). The precipitate was filtered, washed with ether, then solids were dissolved in the EtOAc/THF 2:1 mixture (10 ml) and washed with 10% aq solution of sodium bicarbonate, water, and brine. The solution was dried over sodium sulfate, concentrated in vacuo to dryness, and recrystallized from ether to afford a free base form of compound 49 (0.030 g, 40%). Final product purity was confirmed by NMR and HPLC (C18 column, acetonitrile 5% to 87% over 10 min, retention time 6.80 min). ¹H-NMR (400 MH, DMSO-d6): δ (ppm) 12.18 (br., 1H), 11.07 (br, 1H), 8.56-8.90 (m, 2H), 7.66-7.95 (m, 2H), 727-7.57 (m, 2H, 4.96 (s, 2H), 3.21-3.29 (m, 4H), 2.74 (s, 2H), 2.18-2.41 (m, 4H). MS (ESI) m/z 4753 [MH]+.

### Example 11. Synthesis of 3,9-difluoro-12-(2-piperidin-1-ylethyl)-12,13-dihydro-5H-indolo[2,3-a]pyrrolo[3,4-c]carbazole-5,7(6H)-dione, compound 51

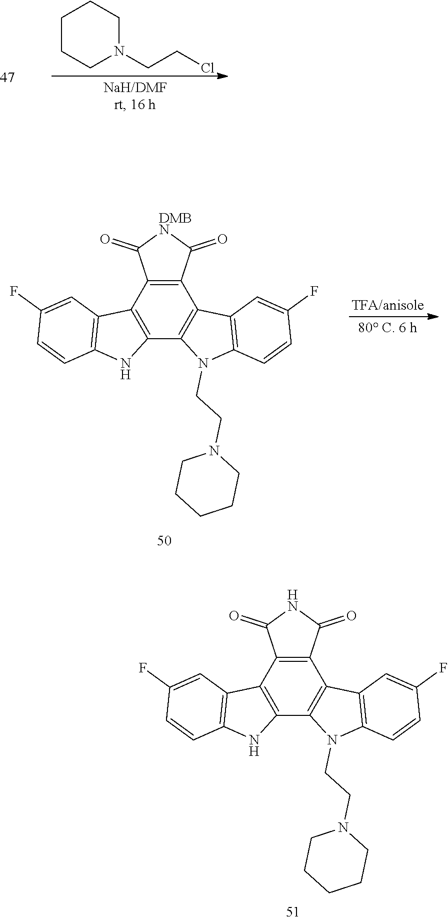

Steps 1-2. Synthesis of 3,9-difluoro-12-(2-piperidin-1-ylethyl)-12,13-dihydro-5H-indolo[2,3-a]pyrrolo[3,4-c]carbazole-5,7(6H)-dione, compound 51. 1-(2-Chloroethyl)piperidine hydrochloride (5.00 g, 27.0 mmol) was dissolved in water (5 ml) and to the stirred solution ether (10 ml) was added. To a vigorously stirring resulting mixture a solution of KOH (1.50 g, 27.0 mmol) in water (5 ml) was added dropwise over 5 min at rt. The organic layer was separated, and the aqueous layer was extracted with ether (2×10 ml). The combined organic extracts were washed with water, brine, dried over sodium sulfate, and evaporated in vacuo to a constant residual volume yielding crude 1-(2-chloroethyl)piperidine (2.5 g, 62%) as a light yellow oil. The product was used in next synthesis without purification.

To a solution of compound 47 (0.19, 0.4 mmol) in DMF (5 ml) a 60% sodium hydride (0.047 g, 1.2 mmol) was added. The resulting dark mixture was stirred at ambient temperature for 1 h, then 1-(2-chloroethyl)piperidine (0.11 g, 0.8 mmol) was added. The reaction mixture was stirred at rt for 16 h, and then the reaction mixture was poured into an ice-cold aq 10% citric acid (20 mL). The resulting mixture was extracted with EtOAc (2×10 ml), the organic layer was washed with water, brine, dried over sodium sulfate and evaporated in vacuo to dryness. The residue was purified by column chromatography (silica gel, eluent 100% DCM to 100% EtOAc) to afford compound 50 (0.1 g, 50%). This product was dissolved in a mixture of anisole/TFA 1:1 (2 ml), the solution was stirred in a microwave reactor at 130° C. for 5 min. The resulting mixture was cooled to it and diluted with ether (5 ml). The precipitate was filtered and washed with ether. The solids were dissolved in a mixture of EtOAc/THF 2:1 (10 ml), washed with aq 10% sodium bicarbonate, water, and brine. The organic solution was dried over sodium sulfate, evaporated in vacuo to dryness and residue was purified by column chromatography (silica gel, eluent 100% DCM to DCM/THF 9:1) to afford the free base form of compound 51 (20 mg, 32%). Final product purity was confirmed by NMR and HPLC (C18 column, acetonitrile 5% to 87% over 10 min, retention time 6.84 min). ¹H-NMR (400 MHz, DMSO-d6): δ (ppm) 12.69 (br, 1H), 11.05 (s, 1H), 8.56-8.97 (m, 2H), 7.66-7.98 (m, 2H), 7.27-7.57 (m, 2H), 4.88 (s, 2H), 2.75 (s, 2H), 2.20-2.42 (m, 4H), 1.14-1.39 (m, 6H). MS (ESI) m/z 473.3 [MH]+.

### Example 12. Synthesis of 12-{2-[(2R,6S)-2,6-dimethylpiperidin-1-yl]ethyl}-3,9-difluoro-12,13-dihydro-5H-indolo[2,3-a]pyrrolo[3,4-c]carbazole-5,7(6H)-dione, compound 53

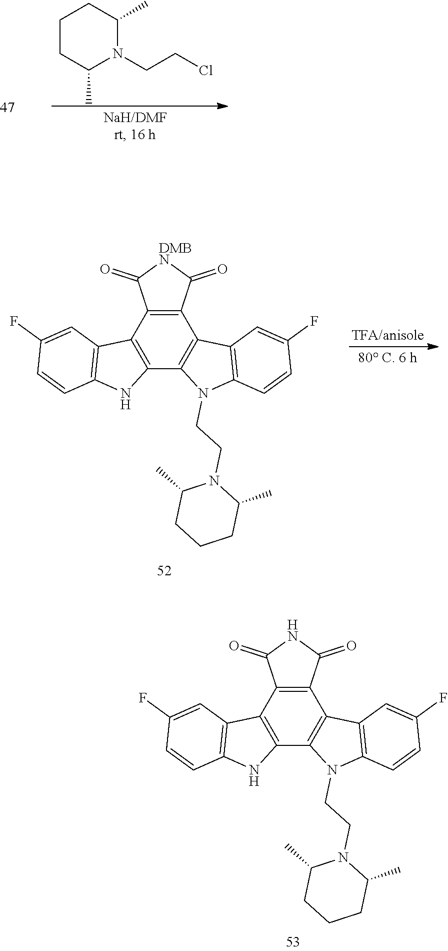

Steps 1-2. Synthesis of 12-{2-[(2R,6S)-2,6-dimethylpiperidin-1-yl]ethyl}-3,9-difluoro-12,13-dihydro-5H-indolo[2,3-a]pyrrolo[3,4-c]carbazole-5,7(6H)-dione, 53. To a solution of compound 47 (0.19 g, 0.4 mmol) in DMF (5 ml) a60% sodium hydride (0.047 g, 1.2 mmol) was added. The resulting dark mixture was stirred at ambient temperature for 1 h, then (2R,6S)-1-(2-chloroethyl)-2,6-dimethylpiperidine (0.13 g, 0.8 mmol) was added. The reaction mixture was stirred at rt for 16 h, and then was poured an ice-cold aq 10% citric acid (20 mL). The resulting mixture was extracted with EtOAc (2×10 ml), the organic layer was washed with water, brine, dried over sodium sulfate, and evaporated in vacuo to dryness. The residue was purified by column chromatography (silica gel, eluent 100% DCM to 100% EtOAc) to afford compound 52 (0.12 g, 53%). This product was dissolved in a mixture of anisole/FA 1:1 (2 ml), the solution was stirred in a microwave reactor at 130° C. for 5 min. The resulting mixture was cooled to rt and diluted with Et₂O (5 ml). The precipitate was filtered and washed with ether. The solids were dissolved in a mixture of EtOAc/THF 2:1 (10 ml) and washed with eq 10% sodium bicarbonate, water, and brine. The solution was dried over sodium sulfate, evaporated to in vacuo dryness, and the residue was purified by column chromatography (silica gel, eluent 100% DCM to DCM/THF 7:3) to afford 53 (25 mg, 35%), Final product purity was confirmed by NMR and HPLC (C18 column, acetonitrile 5% to 87% over 10 min, retention time 6.78 min). ¹H-NMR (400 MHz, DMSO-d6): δ (ppm) 13.17 (br, 1H), 11.05 (s, 1H), 8.56-8.84 (m, 2H), 7.66-7.91 (m, 2H), 7.19-7.57 (m, 2H), 4.73 (s, 2H), 3.07 (s, 2H), 1.04-1.65 (m, 6H), 0.74 (s, 6H). MS (ESI) m/z 5013 [MH]+.

### Example 13. Synthesis of 2,10-dihydroxy-12-(2-piperidinoethyl)-12,13-dihydro-5H-indolo[2,3-a]pyrrolo[3,4-c]carbazole-5,7(6H)-dione, ZE12-0027A

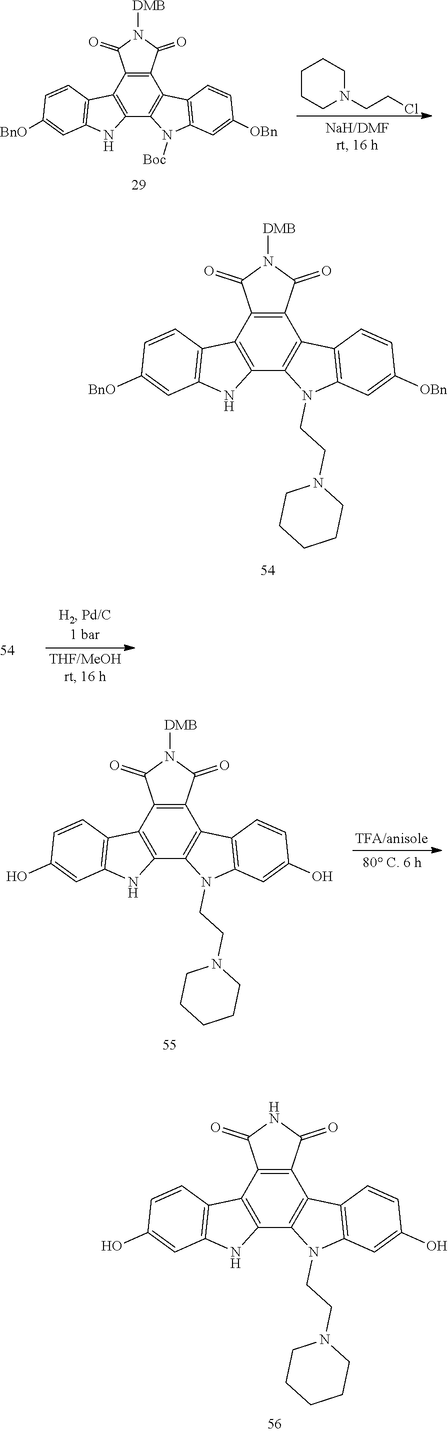

Step 1. Synthesis of 2,10-bis(benzyloxy)-6-(2,4-dimethoxybenzyl)-12-(2-piperidinoethyl)-12,13-dihydro-5H-indolo[2,3-a]pyrrolo[3,4-c]carbazol-5,7(6H)-dione, compound 54 To a solution of tert-butyl 2,10-bis(benzyloxy)-6-(2,4-dimethoxybenzyl)-5,7-dioxo-5,6,7,13-tetrahydro-12H-indolo[2,3-a]pyrrolo[3,4-c]carbazole-12-carboxylate 29 (0.320 g, 0.406 mmol) in DMF (1 ml) a 60% sodium hydride (0.033 g, 0.812 mmol) was added. The resulting dark mixture was stirred at rt for 1 h, then 1-(2-chloromethyl)-piperidine (0.120 g, 0.812 mmol) was added. The reaction mixture was stirred at rt for 16 h, then was poured into ice-cooled 10% aq citric acid (20 ml). The resulting mixture was extracted with EtOAc (2×10 ml), the organic layer was washed with water, brine, dried over sodium sulfate and evaporated in vacuo to dryness. The residue was purified by column chromatography (silica gel, eluent DCM/ether 5:1) to afford compound 54 (0.180 g, 56%) as a yellow solid. Final product purity was confirmed by NMR and HPLC (C18 column, acetonitrile, gradient 40 to 87%, retention time 7.11 min). ¹H-NMR (400 MHz, DMSO-d6): δ (ppm) 12.28 (br, 1H), 8.91 (d, 1H), 8.86 (d, 1H), 7.53-7.58 (m, 6H), 7.36-7.46 (m, 6H), 7.09 (d, 1H), 7.01 (t, 2H), 6.59 (s, 1H), 6.43 (dd, 1H), 5.32 (s, 4H), 5.22 (s, 2H), 4.75 (s, 2H), 3.83 (s, 3H), 3.71 (s, 3H), 3.59-3.66 (m, 2H), 3.43-3.52 (m, 2H), 2.98-3.11 (m, 2H), 1.75-1.92 (m, 5H), 1.33-1.46 (m, 1H). MS (ESI) m/z 799.5 [MH]+.

Step 2. Synthesis of 6-(2,4-dimethoxybenzyl)-2,10-dihydroxy-12-(2-piperidinoethyl)-12,13-dihydro-5H-indolo[2,3-a]pyrrolo[3,4-c]carbazole-5,7(6H)-dione, compound 55, To a solution of compound 54 (0.180 g, 0.225 mmol) in a mixture of THF/MeOH 1:1 (20 ml) a catalytic amount of 10% palladium on carbon (10 mg) was added. The resulting mixture was stirred under hydrogen atmosphere at rt for 16 h. The catalyst was removed by filtration and washed with THF/MeOH. The filtrate was evaporated in vacuo to dryness, and the residue was crystallized in ether to afford 55 (0.100 g, 71%) as a yellow solid. Product purity was confirmed by LCMS (C18 column, acetonitrile, gradient 5 to 87%, retention time 7.13 min), MS (ESI) m/z 799.5 [MH]+.

Step 3. Synthesis of 2,10-dihydroxy-12-(2-piperidinoethyl)-12,13-dihydro-5H-indolo[2,3-a]pyrrolo[3,4-c]carbazole-5,7(6H)-dione, 56. A solution of 55 (0.100 g, 0.162 mmol) in a mixture of anisole/TFA 1:1 (4 ml) was stirred under an argon atmosphere at 7080° C. for 5 h. The resulting mixture was cooled to rt and diluted with ether (5 ml). The precipitate was filtered, washed with ether to afford a TFA salt form of 56 (0.030 g, 40%) as a yellow solid. Final product purity was confirmed by NMR and HPLC (C18 column, acetonitrile, gradient 5 to 87%, retention time 5.12 min). ¹H-NMR (400 MHz, DMSO-d6): δ (ppm) 12.01 (s, 1H), 11.02 (br, 1H), 10.89 (s, 1H), 9.92 (s, 1H), 9.77 (s, 1H), 8.86 (d, 1H), 8.80 (d, 1H), 7.34 (s, 1H), 7.15 (s, 1H), 6.84 (dd, 2H), 5.26-5.31 (m, 2H), 3.65-3.70 (m, 2H), 3.44-3.52 (m, 2H), 3.04-3.11 (m, 2H), 1.74-1.92 (m, 5H), 1.34-1.45 (m, 1H). MS (ESI) m/z 469.4 [MH]+.

### Example 14. Synthesis of 12-{2-[(2R,6S)-2,6-dimethylpiperidin-1-yl]ethyl}-3,9-difluoro-2,10-dihydroxy-12,13-dihydro-5H-indolo[2,3-a]pyrrolo[3,4-c]carbazole-5,7(6H)-dione, compound 59

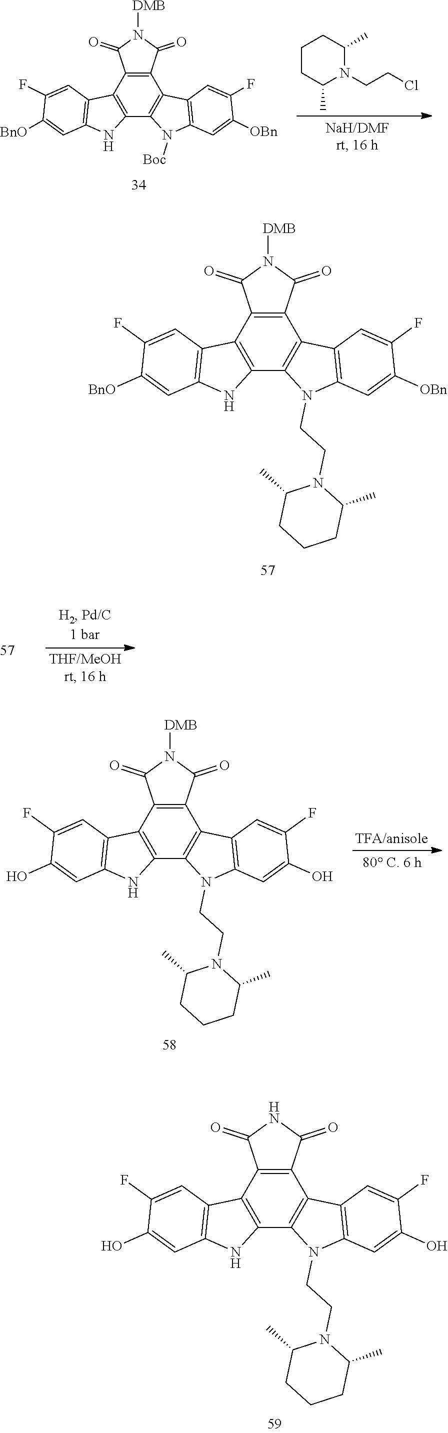

Step 1. Synthesis of 2,10-bis(benzyloxy)-6-(2,4-dimethoxybenzyl)-12-{2[(2R,6S)-2,6-dimethylpiperidin-1-yl]-ethyl)}-3,9-difluoro-12,13-dihydro-5H-indolo[2,3]pyrrolo[3,4-c]carbazole-5,7(6H)-dione, compound 57. To a solution of compound 34 (0.2 g, 1 mmol) in DMF (5 ml) a 60% sodium hydride (0.019 g, 0.48 mmol) was added. The resulting dark mixture was stirred at ambient temperature for 1 h, then (2R,6S)-1-(2-chloroethyl)-2,6-dimethylpiperidine (0.085 g, 0.48 mmol) was added. The reaction mixture was stirred at rt for 16 h, and then was poured into ice-cooled 10% aq citric acid (20 ml). The resulting mixture was extracted with EtOAc (2×10 ml), the organic layer was washed with water, brine, dried over sodium sulfate and evaporated in vacuo to dryness. The residue was purified by column chromatography (silica gel, eluent 100% DCM to 100% EtOAc) to afford crude compounds 57 (150 ng, 72%). ¹H-NMR (400 MHz, DMSO-d6): δ (ppm) 8.55 (d, 2), 7.31-7.65 (m, 12), 7.22 (d, 1H), 6.92 (d, 1H), 6.57 (d, 1H), 6.38 (dd, 1H), 5.36 (s, 2H), 5.33 (s, 2H), 4.58 (s, 2), 4.45-4.55 (m, 2H), 3.83 (s, 3H), 3.69 (s, 3H), 2.90-3.11 (m, 2H), 2.35-2.45 (m, 2H), 0.90-1.59 (m, 6H), 0.68 (s, 3), 0.66 (a, 3H).

Step 2. Synthesis of 6-(2,4-dimethoxybenzyl)-12-{2-[(2R,6S)-2,6-dimethylpiperidin-1-yl]ethyl}-3,9-difluoro-2,10-dihydroxy-12,13-dihydro-5H-indolo[2,3-a]pyrrolo[3,4-c]carbazole-5,7(6H)-dione, compound 58. To a solution of compound 2 (0.150 g, 0.162 mmol) in a mixture of THF/MeOH 1:1 (20 ml) a catalytic amount of 10% palladium on carbon (10 mg) was added. The resulting mixture was stirred under hydrogen atmosphere at rt for 16 h The catalyst was removed by filtration and washed with THF/MeOH. The filtrate was evaporated in vacuo to dryness, and the residue was crystallized in ether to afford 58 (0.100 g, 82%) as a yellow solid.

Step 3. Synthesis of 12-(2-[(2R,6S)-2,6-dimethylpiperidin-1-yl]ethyl)-3,9-difluoro-2,10-dihydroxy-12,13-dihydro-5H-indolo[2,3-a]pyrrolo[3,4-c]carbazole-5,7(6H)-dione, compound 59. A solution of 58 (0.100 g) in a mixture of anisole/TFA 1:1 (2 ml) was stirred at 90° C. for 12 h. The resulting mixture was cooled to ambient temperature and diluted with ether (5 ml). The precipitate was filtered and washed with ether to afford 59 (20 mg, 33%). Final product purity was confirmed by NMR and HPLC(C18 column, acetonitrile 5% to 87% over 10 min, retention time 6.18 min). ¹H-NMR (400 MHz, DMSO-d6): δ (ppm) 11.87 (s, 1M), 11.05 (s, 1H), 10.60 (s, 1H), 10.40 (s, 1H), 9.40 (s, 1H), 8.74 (d, 1H), 8.65 (d, 1H), 7.40 (d, 1H), 7.26 (d, 1H), 5.09-5.43 (m, 2H), 323-3.45 (m, 48), 1.44-2.04 (m, 6H), 1.23 (s, 6H). MS (ESI) m/z 533.4 [MH]+.

### Example 15

### Kinase Assays

Two different assays for measuring kinase inhibitor activity were employed for messing the effectiveness of compounds of the present disclosure for inhibiting PIM kinases. One assay was a standard biochemical assay employing radiolabeled ATP, in which inhibitory concentration of 50% of kinase activity (IC₅₀) was measured by quantifying the amount of radiolabel incorporation into a standard peptide substrate. The second assay used was Perkin Elmer's LANCE® Ultra kinase assay which uses a ULight™-labeled peptide substrate and an appropriate Europium-labeled anti-phospho antibody. When the substrate becomes phosphorylated by a kinase of interest, the phosphorylated site on the substrate is recognized by the Europium-labeled anti-phospho antibody. Upon excitation of the Europium donor fluorophore at 320 or 340 nm, energy is transferred to the ULight acceptor dye on the substrate, resulting in the emission of light at 665 nm (FRET). The intensity of light emission is proportional to the level of ULight peptide phosphorylation. A kinase inhibitor reduces the FRET signal, thus providing an accurate and sensitive measure of inhibition potency.

### Biochemical Kinase Assays

The basic biochemical assay employs radiolabeled ATP to measure the kinase-catalyzed transfer of radioactive phosphorus to a tyrosine-containing peptide substrate, according to the general equation:

Reaction: Substrate+[γ-³³P]−ATP→³³P-Substrate+ADP

The standard protocol used for PIM3 was performed by Reaction Biology Corporation (Malvern, Pa.) using a capture assay performed in 20 mM HEPES at pH 7.5 containing 10 mM MgCl₂, 10 mM MnCl₂, 1 mM EGTA, 0.02% Brij35, 0.02 mg/mL BSA, 0.1 mM Na₃VO₄, 2 mM DTT, 0.02% Brij35, 10 μM ATP, and 20 μM peptide substrate RSRHSSYPAGT. Inhibitors in DMSO were added such that the final concentration of DMSO did not exceed 1%, and the enzyme such that the consumption of ATP was less than 10%. Reagents were combined and incubation at 30° C. for 30 min, the reaction was initiated by adding [γ-³³P]-ATP (10 ρCi/mL [γ-³³P]-ATP) and incubated for 2 h at 30° C. The reaction was then terminated by the addition of one-third volume of stop reagent (0.25 mM EDTA and 33 mM ATP in dH20). A 15 mL aliquot was removed, spotted onto a P-81 filternat ion exchange paper and washed sequentially with 10% (w/v) chloroacetic acid and dH20 to remove ATP. The bound ³³P-peptide substrate was quantified by scintillation counting and the disintegrations per minute (dpm) obtained, being directly proportional to the amount of ³³P-peptide produced by PIM3, were used to determine the IC₅₀ for each compound. Assays for PIM1 and PIM2 were performed analogously, except for the use of peptide substrates KKRNRTLTK for PIM 1 and RSRHSSYPAGT for PIM2.

The inhibitory activity of each compound against PIM kinases was measured with the radioisotope filter binding assay, a type of substrate phosphorylation assays, available at Reaction Biology Corporation (Mavern, Pa.). In this assay involving PIM3 kinase, compounds of this disclosure exhibit IC₅₀ values in the range of 0.1 nM to 2 μM, preferably in the range 0.1 nM to 10 nM. Compound 18 of Example 4 exhibits an IC₅₀ for PIM3 of 025 nM. Positive control inhibitor staurosporine has an IC₅₀ of 0.14 nM vs PIM3 using this assay.

In this assay involving PIM1 and PIM2 kinases, compounds of this disclosure exhibit IC₅₀ in the range of 0.1 nM to 10 μM, preferably in the range 0.1 nm to <10 μM. Compound 18 of Example 4 exhibits an IC₅₀ for PIM1 of 1.8 nM and for PIM2 of 7A nM using this assay. Positive control inhibitor staurosporine has an IC₅₀ of 4.0 nM and 33 nM vs PIM1 and PIM2, respectively, using this assay.

Examples of biochemical inhibition of PIM kinases by compounds 18 and 19 with IC₅₀ values are shown in Table 1.

TABLE 1
Biochemical inhibition of PIM Kinases by
Compounds 18 and 19 at 10 μM ATP

| | IC₅₀ (nM) | | |
|---|---|---|---|
| Compound | PIM1 | PIM2 | PIM3 |
| 18 | 1.8 | 7.4 | 0.25 |
| 19 | 0.98 | 6.7 | 0.20 |

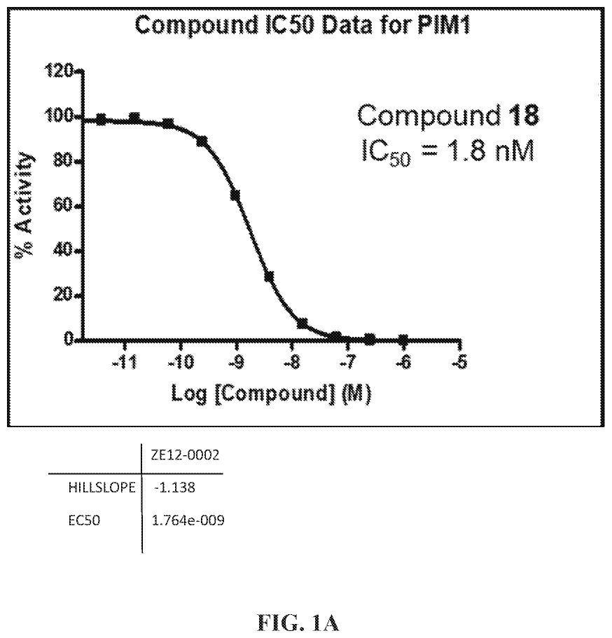

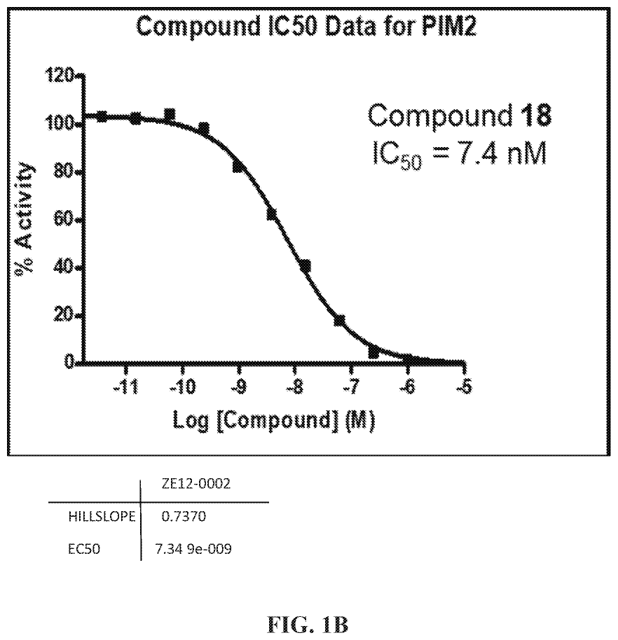

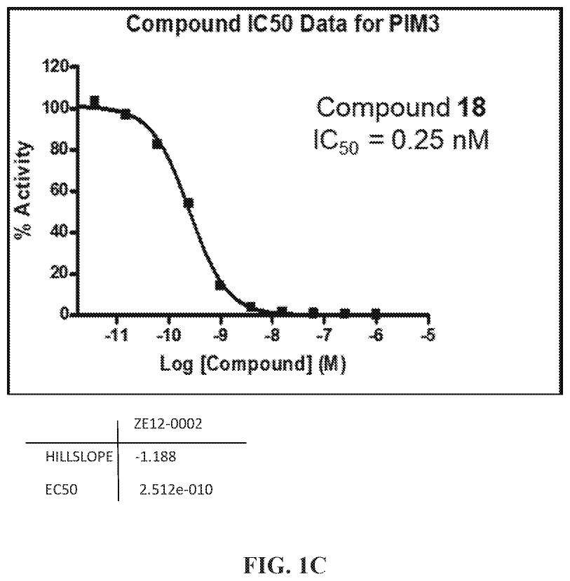

IC₅₀ plots for Compound 18 targeting PIM1-3 using the biochemical assay are provided in FIGS. 1A, 1B and 1C.

### LANCE® Ultra Kinase Assays

The assay was performed in 384-well plates in two steps:
PIM kinases were mixed with the ULight-substrate (CREBtide) in buffer and incubated for 10 min.
Subsequently, ATP and molecules of the present disclosure were added in a kinase buffer at the appropriate concentration and the reactions were incubated for 1 hour, at room temperature in the dark.
After 1 h EDTA was added to stop the kinase reaction, Eu-W1024-labeled anti-phosphoserine-CREB (Ser133) Antibody (anti-phosphoCREBtide specific antibody) was added to the reaction in LANCE Detection buffer. The mixture was allowed to incubate for 30 min to allow binding of the antibody to the phosphorylated site before the plate was read using a Tecan microplate reader

| Reagents Used | [Assay Concentration] |
|---|---|
| PIM1 | 15 pg/ul (0.15 nM) |
| PIM2 | 30 pg/ul (0.45 nM) |
| PIM3 | 15 pg/ul (0.15 nM) |
| CREBtide | 50 nM |
| Eu-labeled anti- | 0.2 nM |
| phosphoserine Ab | |
| ATP | 100 μM−1 mM |

### Tecan Measurement Parameters,

Excitation filter: 325 nm (BW 20 nm)
Emission filter 1: 616 nm (BW 12 nm)
Emission filter 2: 665 nm (BW 12 nm)

### Gain: 100

### Z-position: 23800

### Lag-time: 60 us

Integration time: 500 us

### Materials Used

Plates 384 well, white Corning low volume (#3674)
Tecan Infinite M1000 microplate reader (Thermo Fisher, Waltham, Mass.)
Multichannel pipette 5-120 μl, 0.2-10 μl

### Freezer −20° C.

P30-384FX pipette tips (Beckman)

### Biomek FX Laboratory Automated Workstation

### Reagents

PIM1 kinase 10 ug (#0186-0000-1; ProQinase, Freiburg, Germany)
PIM2 kinase 10 ug (#0223-0000-1; ProQinase, Freiburg, Germany)
PIM3 kinase 10 ug (#P37-10BG; SignalChem, Richmond, Canada)
LANCE® Ultra ULight™-CREBtide 5 mmoles (#TRF0107-M; PerkinElmer, Waltham, Mass.)
ATP (#A1388; Sigma, St. Louis, Mo.)—100 mM stock
Eu-W1024-labeled anti-phosphoserine-CREB (Ser133) Antibody (#TRF0200-D; PerkinElmer, Waltham, Mass.)

### Kinase Assay Buffer—50 mM HEPES pH7.0.1 mM EGTA, 10 mM MgCl₂, 0.01% BRU-35.

The inhibitory activity of each compound against PIM kinases was measured with the LANCE® Ultra Kinase Assays by ChemDiv, Inc. (San Diego, Calif.). Two known pan-PIM inhibitors, PIM447 and AZD-1208 (both purchased from Selleckchem, Houston, Tex., USA) were used as positive controls. ATP Km values for PIM1, PIM2, and PIM3 measured using this assay were: PIM1=170 μM, PIM2=4 μM, PIM3=17 μM. Examples of PIM kinase inhibition using compounds of this disclosure as measured by the LANCE® Ultra Kinase Assays are shown in Table 1 with IC₅₀ values and ATP concentration used.

TABLE 2
IC₅₀ values for PIM Kinase Inhibition using LANCE assay

| | IC₅₀ ((nM) | | | |
|---|---|---|---|---|
| Compound | PIM1 | PIM2 | PIM3 | ATP (mM) |
| AZD-1208 | 0.24 | 15 | 0.44 | 0.1 |
| PIM447 | 0.04 | 0.41 | 0.11 | 0.1 |
| PIM447 | 0.04 | 0.80 | 0.53 | 1 |
| 18 | 1.7 | 41 | 9.5 | 1 |
| 19 | 0.82 | 4.9 | 4.3 | 1 |
| 19 | 0.23 | 3.2 | 1.0 | 0.1 |
| 23 | 0.26 | 0.24 | 1.34 | 1 |
| 23 | 0.23 | 39 | 0.21 | 0.1 |
| 24 | 0.33 | 0.23 | 2.15 | 1 |
| 24 | 0.32 | 5.0 | 0.21 | 0.1 |
| 32 | 2.0 | 480 | 4.9 | 1 |
| 32 | 0.81 | 36 | 1.2 | 0.1 |
| 37 | 0.93 | 180 | 5.0 | 1 |
| 37 | 0.70 | 13 | 1.1 | 0.1 |
| 41 | 0.42 | 674 | 1.6 | 1 |
| 42 | 0.16 | 18 | 1.9 | 1 |
| 49 | 4 | >1000 | 0.7 | 0.1 |
| 51 | 3 | >1000 | 0.8 | 0.1 |
| 53 | 3 | >1000 | 2.4 | 0.1 |
| 56 | 0.7 | 50 | 1 | 0.1 |
| 59 | 0.1 | 37 | 3 | 0.1 |

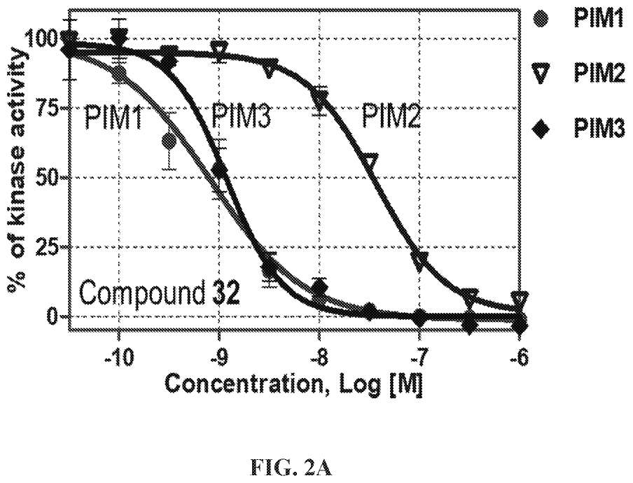

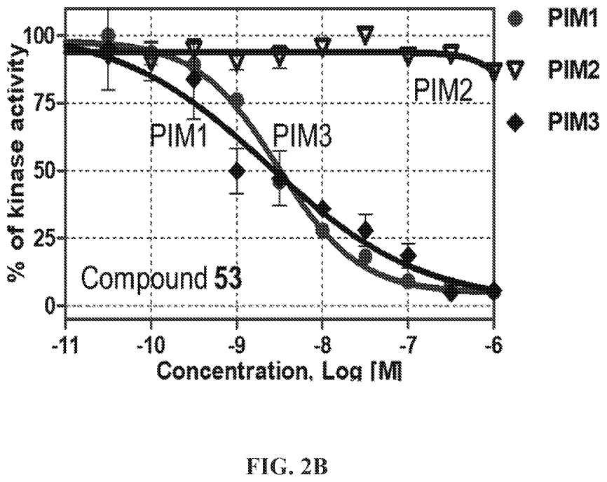

Stacked IC₅₀ plots of Compounds 32 and 53 targeting PIM1-3 using LANCE assay at 0.1 mM ATP are provide in FIGS. 2A and 2B.

### Example 16

### Cell-Based Growth Inhibition for PNM Inhibitors

Cancer cell lines used for growth and proliferation inhibition assays are obtained from commercial sources (Life Technologies, Carlsbad, Calif. or ATCC, Monasses, Va.), except for liver cancer cell line Huh7, which was purchased from RIKEN (Tokyo, Japan). All cell lines were stored and maintained in recommended media containing 10% fetal bovine serum (Thermo Fisher, Waltham, Mass.) according to provider's instructions.

Cancer cell lines used to screen PIM inhibitors for activity include:

### Pancreatic: MIA PaCa-2, PANC-1, Capan-1, PSN1, and JOPACA-1

### Colorectal: Caco-2, COLO 320, DLD-1, HCT-15, HCT-116, HT-29, and SW48

### Gastric: AGS, SNU-1, SNU-5, SNU-16, Hs 746T, NCI-N87, KATO III, HGC-27, MNK28, MNK45

### Hepatic: HepG2, C3A, HuH7, Hep3B, HLE, HepaRG, HLF, SK-Hep1, PLC/PRF/5

### Prostate: DU-145, PC-3 and LNCaP, LAPC-4, LAPC-9, and VCaP

Ewing's sarcoma: A673, TC-71, RD-ES, A4573, Hs 822T, Hs 863T

### Cancer Cell Growth Inhibition Data:

Compounds of this disclosure were tested for growth inhibition against cancer cell lines, including HepG2, Huh7, HepRG, A673, and DU-145, using the CELL TITER-GLO® 2.0 Luminescent Cell Viability Assay (Promega Corporation, Madison, Wis.). The CellTiter-Glo® Luminescent Cell Viability Assay is a sensitive homogeneous method to determine the number of viable cells in culture, Detection is based on using the luciferase reaction to measure the amount of ATP from viable cells. The amount of ATP in cells correlates with cell viability. Within minutes after a loss of membrane integrity, cells lose the ability to synthesize ATP, and endogenous ATPases destroy any remaining ATP; thus the levels of ATP fall precipitously. The CellTiter-Glo® Reagent does three things upon addition to cells. It lyses cell membranes to release ATP; it inhibits endogenous ATPases, and it provides luciferin, luciferase and other reagents necessary to measure ATP using a bioluminescent reaction. The unique properties of a proprietary stable luciferase. There is a linear relationship (r2=0.99) between the luminescent signal and the number of cells from 0 to 50.000 cells per well.

### Reagents Used

DMEM (Paneco, cat #C420)
Williams Medium (Gibco, cat #12551032)
Fetal bovine serum, FBS (HyClone, cat # SH 30094.03)
Pen-Strep (Paneco, cat # A065)
MEM non-essential amino acids (Paneco, cat # F115)
L-glutamine (Paneco, cat # F032)
Sodium pyruvate (Paneco, cat # F023)
Versen (Paneco, cat # P080)
Accutase (Innovative Cell Technologies, Inc., cat, # AT104)
DMSO (Panreac, cat. #141954.1611)
The CellTiter-Glo® Luminescent Cell Viability Assay (Promega, cat. # G7573)

### Equipment and Materials Used

Biomek 384 FX Laboratory Automated Workstation (Beckman Coulter Inc., Fullerton, Calif.)
Biomek 2000 Laboratory Automated Workstation (Beckman Coulter Inc., Fullerton, Calif.)
Microscope Axiovert 40
Microbiological safety cabinet, classII (NuAire, USA)
CO₂ incubator (VWR Science, USA)
Bright line Hemacytometer (Z359629, Sigma, Ill., USA)
Tecan Infinite M1000 microplate reader (Thermo Fisher, Waltham, Mass.)
Plates: 384-well black/clear, tissue culture treated, flat bottom (Falcon, #353962)

### Cell Lines

Huh7
HepG2
A673
DU-145
HepaRG

TABLE 3
Cell lines used in growth inhibition assay.

| | ATCC | Cells per | |
|---|---|---|---|
| Cell line | Number | well | Culture medium |
| Huh7, HepG2, | | 2000-3000 | DMEM + 10% FBS, 2 mM |
| A673, DU-145 | | | glutamine, NEAA + Na |
| | | | Pyrivate |
| HepRG | | 2000 | Williams + 10% FBS, 2 mM |
| | | | glutamine, NEAA + Na |
| | | | Pyrivate |

All assays were performed by Reaction Biology Corporation (Malvern, Pa.) or ChemDiv, Inc. (San Diego, Calif.). All cell lines were purchased from American Type Culture Collection (ATCC, Manassas, Va.), except Huh 7 (RIKEN, Toyo, Japan) and HepaRG (Life Technologies, Carlsbad, Calif.). Cell lines were maintained in the recommended culture media in the presence of 10% fetal bovind serum, 100 μg/mL of penicillin and 100 μg/mL of streptomycin at 37° C. in a huminidifed atmosphere of HEPA-filtered 5% CO₂ and 95% air.

### Cell Prorogation

Conditions: 37° C., air 95%; HEPA-filtered carbon dioxide (CO2) 5%, humidified atmosphere.
Cells were grown in 175 cm² flasks to 80-90% of confluency.
Culture media was aspirated and cell layer was briefly rinsed with Versen solution to remove all traces of serum.
2 mL of accutase was added to cells.
Flasks were retuned to incubator for 5 min to allow cell detachment.
Add 6.0 mL of complete growth medium.
Single cell suspension was created by gently pipetting.
Cells were counted using a Hematocytometer and a suspension with the desired cell concentration was prepared.

### Cell Plating

Single cell suspensions were prepared as described above, cells were recounted and resuspended to final density.
Cells were plated in a 384-well optical bottom plates by Biomek 384 FX, with 40 μL of cell suspension in each well.
Assay plates were centrifuged at 100 rpm for 1 minute and then kept at 37° C., 5% CO₂, humidified atmosphere for 24 hours prior to treatment.

To initiate the growth inhibition study, compounds of this disclosure and reference compounds were dissolved in DMSO solution to create 10 mM stock solutions. Stock solutions (70 mL) were diluted with cell culture media to produce 500× serial dilutions and 10 mL of final compound dilutions were added to cells in assay plates to screen a series of 10 doses starting at 100 mM. Final DMSO concentration was 0.2%. Assay plates are centrifuged at 100 rpm, 1 minute and kept at 37° C., 5% CO₂, humidified atmosphere. After 72 hours of incubation with compounds, 10 μL CellTiter-Glo Reagent was added to assay plates using the Biomek 384 FX. Luminescence intensity was measured for each well using the Tecan M 1000 microplate reader after 5 min of incubation with CellTiter-Glo Reagent. The number of viable cells in culture was determined based on quantitation of the ATP present in each culture well. Experimental data was calculated as percent growth inhibition by dividing luminescence values from treated wells by the average luminescence values from untreated control wells and subtracted from 100. The EC₅₀ value was defined as the drug concentration needed to inhibit 50% of the cell growth compared to growth of the untreated control cells. The EC₅₀ curves were plotted and EC₅ values were calculated using the GraphPad Prism 4 program based on a sigmoidal dose-response equation. Table 4 lists the EC₅₀ values for compounds of this disclosure vs tested cancer cell lines.

TABLE 4
EC₅₀ values for Compounds of this disclosure
and Reference Compounds

| | EC₅₀ (nM) | | | | |
|---|---|---|---|---|---|
| Compound | HepG2 | Huh7 | HepaRG | DU-145 | A673 |
| Staurosporine | 46 | NA | NA | 4.8 | NA |
| Sorafenib | 2300 | 2600 | 1400 | NA | 18000 |
| Regorafenib | 4300 | 4400 | 2300 | NA | 30000 |
| AZD-1208 | 27950 | 13540 | NA | NA | 19700 |
| PIM447 | 28260 | 2500 | 8400 | NA | 23200 |
| 18 | 76 | 41 | NA | 41 | NA |
| 19 | 34 | 80 | 48 | 40 | 10 |
| 23 | 85 | 50 | NA | 11 | NA |
| 24 | 25 | 14 | NA | 13 | 6.2 |
| 32 | 570 | 240 | 250 | NA | 37 |
| 37 | 54 | 77 | 16 | NA | 2.2 |
| 41 | NA | 100 | NA | NA | 4.4 |
| 42 | NA | 100 | NA | NA | 5.0 |
| 49 | 40 | 100 | >10000 | NA | 57 |
| 56 | 315 | 484 | 77 | NA | 35 |
| 59 | 266 | 925 | 20 | NA | 3.1 |

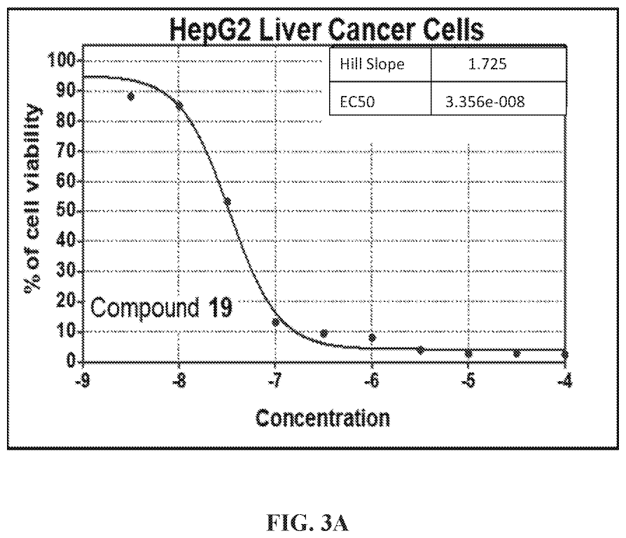

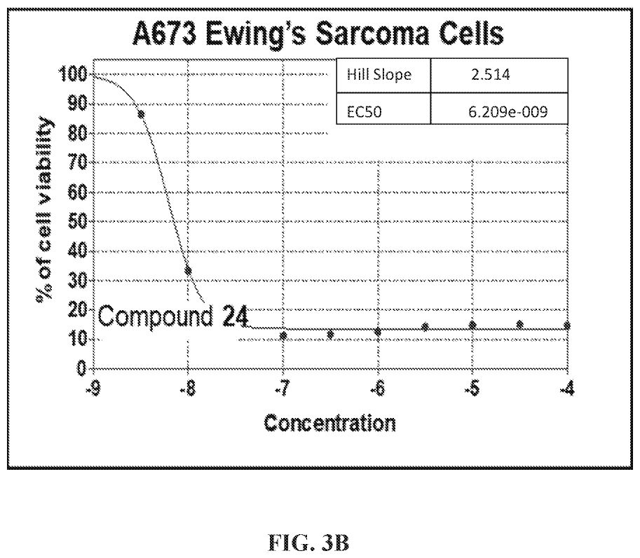

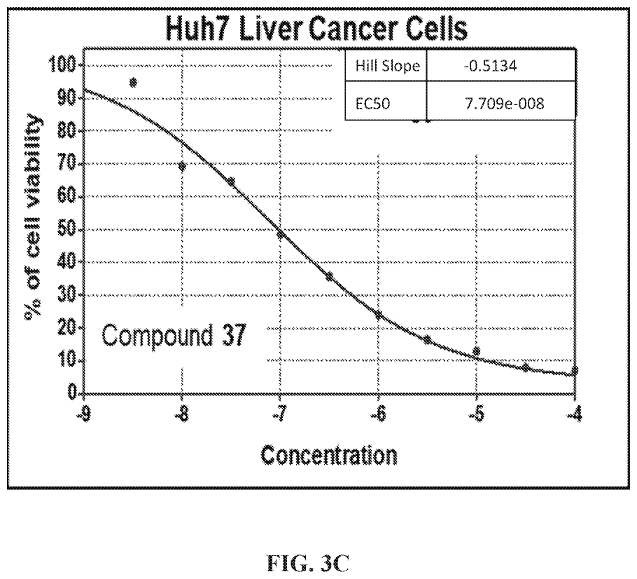

NA = Not assayed
EC₅₀ plots showing cancer growth inhibition by compounds 19, 24 and 37 are provided in FIGS. 3A, 3B and 3C.

### Example 17

Treatment of Liver Cancer by Administration of a PIM Inhibitor Compound Disclosed Herein in an Animal Model

Animal models are used to examine the ability of a compound of this disclosure to ameliorate the growth of liver cancer. Inhibitors of this disclosure ae applied in mice where liver tumors have been induced through xenografts created by the transfer of liver cancer cells, such as Hep2G or Huh7, into the subcutaneous or intraperitoneal space of a mouse, or through production of a human-mouse liver tumor xenograft.

Mice. Twelve female BALB/c mice are purchased from The Jackson Laboratory (Bar Harbor, Me., USA). Animals are maintained in accordance with the Guide for the Care and Use of Laboratory Animals. All study protocols are approved by the institutional Animal Care and Use Committee.

Compounds: Compounds 24 and 37 are prepared for in vivo intraperitoneal administration in PBS vehicle (phosphate-buffered saline: ThermoFisher Scientific Inc., Waltham, Mass.) containing 0.5% methylcellulose/0.025% Tween 20 (Sigma-Aldrich, St. Louis, Mo.).

### Liver Cancer Xenograft Mice

HepG2 human liver cancer cells are suspended in Hank's balanced salt solution (HBSS) (2×10⁷ cells/mL), and the suspension (100 μL) is subcutaneously injected into the back of twenty-one female BALB/c mice. Mice are maintained for 15 days following injection of HepG2 cells, and then randomly split into three groups of six treatment mice and one control mouse per group. Ater establishment of the nude mice xenograft model, tumor dimensions are measured every 3 days using micrometer calipers. Compound 24 (25 mg/kg) is administered to the 6 treatment mice in each group intraperitoneally, while the control mice in each group receives vehicle only, once per day (q.d.) for 5 consecutive days, followed by 2 days with no injections, and the cycle is then repeated three times. Mice are weighed daily, starting from the date of HepG2 cell injection (day 0). At 43 days after subcutaneous inoculation with HepG2 cells, mice are euthanized via carbon dioxide asphyxiation and all tumors are excised immediately following death, weighed and measured, and then snap-frozen in liquid nitrogen. Tumor volume (TV) is calculated by the following formula; TV=0.5ab², where a is tumor length in mm, and b is tumor width in mm. Compounds of this disclosure are shown to reduce tumor size from 0%-88% relative to control tumors and mouse body weight is maintained within 10% of day 0 baseline.

### Immunofluorescence Analysis of Apoptotic Cells in Xenograft Specimens

Six serial sections (5-μm thick) are obtained for each frozen tumor, mounted on glass slides, and then fixed in 1% paraformaldehyde. Terminal deoxynucleotidyl transferase-mediated nick end labeling-based TUNEL assay for apoptosis detection is performed on four sections using the In Situ BrdU-Red TUNEL assay kit according to the manufacturer's instructions (Abeam, Cambridge, UK). Two tissue sections processed in the absence of terminal deoxynucleotidyl transferase serve as negative controls. The fluorochrome-conjugated anti-BrdU-Red antibody is excited using a 490-nm bandpass filter with emission collected at 576 nm.

Fluorescence microscopy is performed using a 40× objective (Zeiss Plan-Neofluar) on an Olympus Eclipse TE2000—S inverted phase microscope (Olympus, Melville, N.Y., USA). Images are analyzed using Image-Pro Plus software version 4.0. The apoptosis-positive cell numbers in each animal are determined in 10 randomly chosen fields at 400× magnification by an examiner blinded to the experimental procedures. Four sections of the same tumor and four tumors per group are analyzed. Tumors are traced manually with reference to the parallel H&E sections so as to exclude edges and necrotic and nonmalignant tissues from analysis. Apoptotic nuclei, often consisting of clusters of discrete nuclear fragments, can be readily defined using image analysis criteria so as to reject artifacts. The extent of apoptosis in each tumor, expressed as proportional area, is calculated from the sum of the TUNEL-positive pixel area divided by the total viable tumor area.

Statistical analysis. Mean values and standard deviations are calculated for all parameters measured. Mouse weights and tumor weights and volumes between each group are compared using a paired Student's t-test and are reported as mean standard deviation. Comparisons between control and treatment groups are made and statistical significance is evaluated by one-way ANOVA, followed by the Tukey-Kramer test, using SPSS 10 software (IBM, Inc., Chicago, Ill., USA). P-values <0.05 were considered to indicate a statistically significant result.

### Hepa1-6 Liver Cancer Xenograft Mice

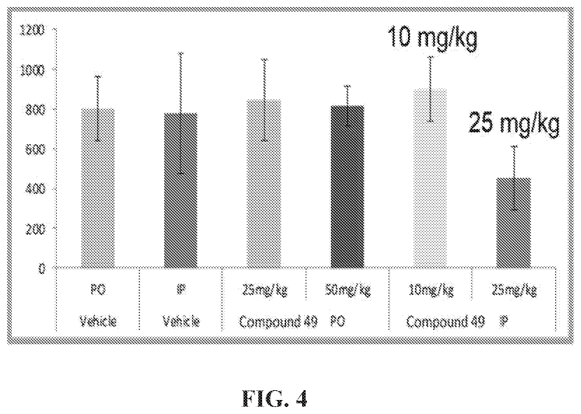

Hepa1-6 mouse liver cancer cells are suspended in Hank's balanced salt solution (HBSS)(2×10⁷ cells/mL), and the suspension (100 μL) was subcutaneously injected into the back flank of six female CD57 immunocompetent mice. After growing for 21 days, the tumors were resected, cut into small pieces, and inserted under the skin on each back flank of twelve female CD57 mice (24 tumors total). Mice were maintained for 15 days following tumor insertion to establish robust and consistent tumor growth. The mice were randomly split into six groups of two mice each (two tumors per mouse). Alter establishment of the Hepa1-6 xenografts in CD57 mice, tumor dimensions were measured every 2 days using micrometer calipers. Four groups were treated with compound 49 (PO: 25 mg/kg and 50 mg/k; IP: 10 mg/kg and 25 mg/kg) by both oral (PO, gavage) and intraperitoneal (IP) administration once per day (q.d.) and two control groups were administered vehicle only through the same routes for 10 consecutive days. Mice are weighed daily, starting from the date of Hepa1-6 tumor insertion (day 0). At 25 days after subcutaneous inoculation with Hepa1-6 tumors, mice were euthanized via carbon dioxide asphyxiation and all tumors were excised immediately following death, weighed and measured, and then snap-frozen in liquid nitrogen. Tumor volume (TV) was calculated by the following formula: TV=0.5ab², where a is tumor length in mm, and b is tumor width in mm. Relative tumor volume data from this study is shown in FIG. 4. Compound 49 of this disclosure was shown to reduce tumor size up to 50% relative to control tumors when administered by IP injection using a dose of 25 mg/kg. No tumor reduction was observed at lower doses or upon PO dosing in this study. Body weight of mice in all groups was maintained within 10% of day 0 baseline.

### Formulation Examples

### Example 18

### Parenteral Formulation

To prepare a parenteral pharmaceutical composition of the compounds of this disclosure that are suitable for administration by injection, the compounds can be formulated as a mixture and incorporated into a dosage unit form. By way of example, a typical 5 mg/ML parenteral formulation of a compound of this disclosure proportionally contains, in addition to the compound itself (0.5%), propylene glycol (40%), ethyl alcohol (10%), sodium benzoate/benzoic acid (5%), benzyl alcohol (1.5%), and water (43%).

### Example 19

### Oral Microemulsion Formulation

To prepare a pharmaceutical composition of the compounds of this disclosure that are suitable for oral administration, the compounds can be formulated as a mixture and incorporated into a dosage unit form. By way of example, a typical 25 mg oral (capsule) formulation of a compound of this disclosure contains, in addition to the compound itself, polyoxyl 40 hydrogenated castor oil, gelatin, polyethylene glycol 400, glycerin 85%, dehydrated alcohol, corn oil mono-di-triglycerides, titanium dioxide, vitamin E, ferric oxide yellow, ferric oxide red, carmine, hypromellose 2910, propylene glycol, and purified water.

### Example 20

### Oral Solid Dosage Formulation

To prepare a pharmaceutical composition of the compounds of this disclosure that are suitable for oral solid dosage (tablet) administration, the compounds can be formulated as a mixture and incorporated into a dosage unit form. By way of example, atypical 50 mg oral solid dosage formulation of a compound of this disclosure can be prepared by granulating and compacting into a solid mixture that contains, in addition to the compound itself, excipients, binders and fillers that include modified starch, polyethylene glycol 400, stearyl citrate, polyvinylpyrrolidone, lecithin, mannitol, sorbitol, sage extract, calcium phosphate and gelatin.

### Example 21

Sublingual (Hard Lozenge) Composition

To prepare a pharmaceutical composition for buccal delivery, such as a hard lozenge, mix 100 ng of a compound of this disclosure with 420 mg of powdered sugar mixed, and then with 1.6 mL of light corn syrup, 2.4 mL distilled water, and 0.42 mL mint extract. Gently blend the mixture and pour into a mold to form a lozenge suitable for buccal administration.

### Example 22

Fast-Disintegrating Sublingual Tablet

A fast-disintegrating sublingual tablet can be prepared by mixing 48.5% by weight of a compound of a compound of this disclosure together with 44.5% by weight of microcrystalline cellulose (KG-802), 5% by weight of low-substituted hydroxypropyl cellulose (50 gm), and 2% by weight of magnesium stearate. The formulation can be prepared by mixing the amount of compound of Formula (I) or (IV)-(VI) with the total quantity of microcrystalline cellulose (MCC) and two-thirds of the quantity of low-substituted hydroxypropyl cellulose (L-HPC) by using a three-dimensional manual mixer (INVERSINA®, Bioengineering AG, Switzerland) for 4.5 minutes. All of the magnesium stearate (MS) and the remaining one-third of the quantity of L-HPC are added 30 seconds before the end of mixing. Tablets are prepared by direct compression (AAPS Pharma Sci Tech., 2006; 7(2):E41). The total weight of the compressed tablets is maintained at 150 mg.

1-28. (canceled)

29. A compound having the structure of Formula (IIA) or Formula (IIB): 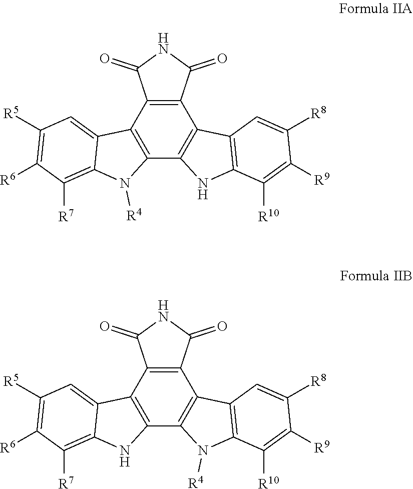
wherein each R4 is selected from the group consisting of 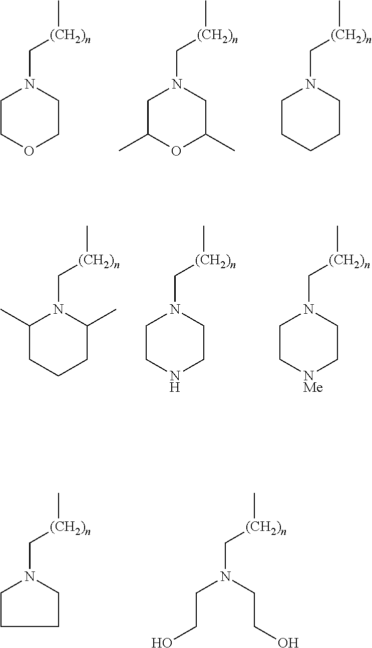
where n=0-5,
each R5, R6, R7, R8, R9, and R10 is the same or different and independently selected from H, halogen, —N3, —CN, —NO2, —OH, —OCF3. —OCH2F, —OCF2H, —CF3, —CF2H, —SR1, —S(═O)R2, —S(═O)2R2, —OS(═O)2F, —OS(═O)2(OR2), —S(═O)2(OR2), —NR3S(═O)2R2, —S(═O)2N(R3)2, —OC(═O)R2, —CO2R3, —N(H)R3, —N(R3)2, —OR3, —NR3C(═O)R2, —NR3C(═O)OR3, —NR3C(═O)N(R3)2, CH2NH2, —CH2N(R3)2, —CH2SR1, —C(═O)NH2, —C(═O)N(R3)2, —C(═O)R3, substituted or unsubstituted alkyl, substituted or unsubstituted alkenyl, substituted or unsubstituted alkynyl, substituted or unsubstituted alkoxy, substituted or unsubstituted heteroalkyl, substituted or unsubstituted cycloalkyl, substituted or unsubstituted heterocycloalkyl, substituted or unsubstituted aryl, or substituted or unsubstituted heteroaryl, or an optional substituent selected, for example, haloalkyl, alkenyl, arylalkyl, alkoxyalkyl, hydroxyalkyl, monoalkylaminoalkyl, dialkylaminoalkyl, acylaminoalkyl, acyloxyalkyl, cyanoalkyl, amidinoalkyl, carboxyalkyl, alkoxycarbonylalkyl, aminocarbonylalkyl, aryl, alkylaryl, aminoalkyl, heteroaryl, carbonylalkyl, amidinothioalkyl, nitroguanidinoalkyl, a protecting group, a glycose, aminoglycose or alkylglycose residue.

30. The compound of claim 29 wherein each R4 is selected from the group of 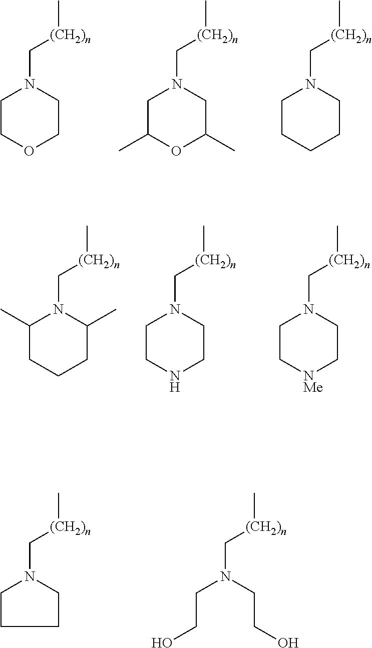
where n=1,
each R5, R6, R8, and R9 is the same or different and independently selected from H, halogen, or —OH, and
each R7 and R10 is H.

31. A compound of claim 29, or a pharmaceutically acceptable salt, solvate, hydrate, N-oxide, prodrug, or isotopic variants thereof, wherein the compound inhibits the catalytic activity of serine/threonine kinases PIM1, PIM2, and/or PIM3.

32. A compound of claim 29, or a pharmaceutically acceptable salt, solvate, hydrate, N-oxide, prodrug, or isotopic variants thereof, wherein the compound selectively inhibits the catalytic activity of one or more PIM kinase.

33. A compound of claim 29, or a pharmaceutically acceptable salt, solvate, hydrate, N-oxide, prodrug, or isotopic variants thereof, wherein the compound selectively inhibits the catalytic activity of PIM3 kinase.

34. A compound according to claim 29 for treating a patient or individual suffering from a malignant disease.

35. A compound according to claim 29 for treating a cancer of the endodermal organs, including but not limited to the cecum, pancreas, liver, stomach, intestine, colon, prostate, thyroid, esophagus, lungs, and gallbladder.

36. A compound according to claim 29 for treating pancreatic cancer, liver cancer, gastric cancer, colorectal cancer, prostate cancer, esophageal adenocarcinoma, squamous cell carcinoma, nasopharyngeal carcinoma, gastric adenocarcinoma, pancreatic ductal adenocarcinoma, hepatocellular carcinoma, gallbladder adenocarcinoma, prostatic adenocarcinoma, colorectal adenocarcinoma, gastrointestinal stromal tumors (GIST), or gastrointestinal carcinoid tumors.

37. A method of inhibiting or partially or selectively inhibiting the activity of PIM kinases comprising contacting the PIM kinases with a compound of claim 29, or a pharmaceutically acceptable salt, solvate, hydrate, or N-oxide, prodrug, or isotopic variants thereof.

38. The method of claim 37, wherein said contacting causes substantially complete inhibition of PIM kinases, contacting causes partial inhibition of PIM kinases, or contacting modulates cancer cell growth and survival.

39. The method of claim 37, wherein inhibiting or partially or selectively inhibiting the activity of PIM kinases with selective inhibition of PIM3 over PIM1 and PIM2 kinases by a factor of 1.5, 10, 100, 1000, or more, further comprising contacting the three kinases, separately or together, in vitro or in vivo, with a compound as in claim 1 or a pharmaceutically acceptable salt, solvate, hydrate, or N-oxide, prodrug, or isotopic variants thereof.
